(19) United States
(12) Patent Application Publication
(10) Pub. No.: US 2024/0264084 A1
(43) Pub. Date: Aug. 8, 2024

(54) SUBSTRATES, METHODS OF PATTERNING THIN FILMS, AND THEIR USE

(71) Applicant: AUCKLAND UNISERVICES LTD, AUCKLAND (NZ)

(72) Inventors: Neil Gregory Raphael BRODERICK, Auckland (NZ); Colin Lee HISEY, Auckland (NZ); Lawrence William CHAMLEY, Auckland (NZ); Mohammadrahim KAZEMZADEH, Auckland (NZ); Miguel Martinez CALDERON, Auckland (NZ); Weiliang (Peter) XU, Auckland (NZ)

(21) Appl. No.: 18/567,895

(22) PCT Filed: Jun. 8, 2022
(86) PCT No.: PCT/NZ2022/050070

§ 371 Date: Dec. 7, 2023

(30) Foreign Application Priority Data
Jun. 8, 2021 (NZ) 776784
Dec. 16, 2021 (NZ) 783607

(51) Int. Cl. G01N 21/65; G06N 3/08

(52) U.S. Cl. CPC G01N 21/658; G06N 3/08

(57) ABSTRACT

Disclosed herein are substrates for surface-enhanced Raman spectroscopy (SERS), methods of fabrication of the same using soft and nanoparticle lithography or laser-induced nano structuring of thin films (LINST), and their use to characterize extracellular vesicles (EVs) from a range of sources including but not limited to cancers, bacteria, viruses and/or placental cells. Also disclosed are machine learning methods for classifying and/or identifying SERS spectra from particles including EVs, the machine learning methods including bottleneck classifiers or layers configured to reduce the dimension of the network. In further methods the bottleneck classifier is combined with an autoencoder in either a supervised or unsupervised manner to identify of classify SERs spectra features.

### FIELD OF THE INVENTION

The present invention relates to a substrate and sensing system capable of classifying biological samples, including bacteria and viruses, identifying diseases, such as cancer, providing data to assist with the diagnosis and of monitoring diseases. In particular, the present invention relates to a substrate for surface-enhanced Raman spectroscopy, methods of fabrication of the same, and a system and method to characterize extracellular vesicles from a range of sources including but not limited to cancers, bacteria, viruses and/or placental cells.

### BACKGROUND

Extracellular vesicles (EVs) are micro and nanoscale lipid-enclosed packages that are derived from the membranes of parental cells and can harbour diverse molecular cargo such as proteins, DNA, RNA, glycolipids, organic small molecules, etc.¹ They have shown potential as liquid biopsy targets for cancer because their structure and contents reflect their cell of origin. However, progress towards the clinical applications of EVs has been hindered due to the low abundance of disease-specific EVs compared to EVs from healthy cells; such applications thus require highly sensitive and adaptable characterization tools.

Raman scattering is an inelastic form of light scattering that can provide information about the chemical bonds present in the scattering material. The obtained spectra can then be used as a unique fingerprint of the material, enabling many successful applications in biological sensing including cancer detection and classification.²⁻⁴ However, conventional Raman spectroscopy requires a relatively abundant sample and long signal acquisition times to provide accurate results due to the low probability of the inelastic scattering. This has hindered the utility of Raman spectroscopy for many applications, particularly in clinical scenarios where sample amounts are limited, and long signal acquisition times are impractical.

Surface-enhanced Raman Spectroscopy (SERS)⁵ is a powerful sensing method capable of increasing the degree of Raman scattering by many orders of magnitude, drastically improving its potential for use in applications involving limited or rare samples. SERS works by exploiting plasmonic resonances at a substrate surface, which can enhance the local intensity of light and thus the amplitude of the Raman signal.^{6,7} Various geometric structures for SERS substrates⁸⁻¹¹ have been investigated.

In the past, two main methods of SERS substrate fabrication have been used: nanoparticles synthesized using bottom-up synthetic methods, self-assembled nanoparticles and nanoarrays from colloidal solution deposition; and direct nanopatterning of solid surfaces.^{12, 13} Colloidal suspension deposition involves the deposition of a layer of nanoparticles onto a surface, mixture within hybrid materials, or direct mixture with EV suspensions which can then either be used directly or as a template for subsequent replica molding, depending on the approach.¹⁴⁻²⁰

To have precise control over the nanostructure geometry of the substrate, most of these fabrication methods use different types of nanometric lithography such as electron-beam lithography (EBL),^{21,22} focused ion beam lithography (FIB) ²³⁻²⁵ and combinations of soft and nanoparticle lithography.²⁶⁻²⁸ Soft and nanoparticle lithography are mostly preferred to their counterparts (EBL, FIB) as they more easily achieve three dimensional structures and do not require sophisticated equipment, making them more accessible and less expensive. For instance, nanometric beads in polydimethylsiloxane (PDMS) have been coated in silver to fabricate three dimensional SERS structures.^{19,28}

While roughening or patterning techniques can be used to create nanostructures on various surfaces, their fabrication often relies on availability of nanometre precision lithography systems within cleanrooms, such as hole-mask colloidal focused ion beam lithography, electron beam lithography, or photolithography.^{12,29-31} Importantly, access to these facilities is often costly and requires additional materials/gases which can severely limit accessibility and routine usage outside of a laboratory research setting. Existing approaches to SERS substrate fabrication suffer from disadvantages such as lack of scalability, complex fabrication methods involving multiple steps, sometimes including masking steps and/or contacting steps which can introduce contamination, and lack of reproducibility.³²⁻³⁶

Some techniques focus on tuning surface wettability of SERS substrates. Femtosecond (fs) laser machining has been used to create combinations of superhydrophobic/philic areas that enhance detection performance by concentrating analyte molecules into small areas, leading to detection of analyte molecules at concentrations as low as 10⁻¹³ M using SERS.³⁷⁻⁴⁰ Additionally, fs-laser induced plasma assisted ablation has been used to create active SERS substrates based on silver (Ag) nanoparticles, demonstrating promising results regarding food safety detection.⁴¹

Other laser-based surface roughening approaches for SERS substrate fabrication mainly focus on the phenomenon known as laser induced periodical surface structures (LIPSS) which allows the fabrication of nanopatterns with a sub-wavelength resolution.⁴²⁻⁴⁴ These efforts have been directed towards LIPSS nanostructuring of base materials, such as silicon or glass, followed by the deposition of a SERS-active film (substrate) of gold or silver.⁴⁵⁻⁵¹

Work exploring the LIPSS pattering of other types of thin films⁵²⁻⁵⁴ has shown there is a risk of completely ablating the thin film due to the use of high intensity pulses, even when using the low powers required for LIPSS formation.

The notion of transformation optics^{7,55} has been applied to investigate the effect of substrate curvature on enhancement of the Raman signal.⁵⁶ A curved gold plate formed by coating polystyrene nanoparticles with gold and then dissolving the polystyrene was transferred to another substrate and silver nanoparticles were added to the gold surface using cluster beam deposition.⁵⁷ Sensitivity down to single molecule detection was achieved, but as a significant portion of this enhancement was due to the plasmonic interaction of silver nanoparticles, it again suffers from weak chemical stability. The fabrication of such structures is also very complicated, and the final mechanical stability is low due to the weak adhesion of the transferred gold layer to the new substrate.

EVs have been investigated as liquid biopsy markers for cancer detection and identification using SERS. Establishing the Raman spectra of cancer-specific EVs may be useful in diagnosis or treatment monitoring. Methods of SERS detection that have been used for the characterization of cancer EVs and other biological applications generally fall into two main categories: labelled, where substrates are functionalized with probes that target specific antigens on the EV surface using antibodies, aptamers, or peptides;^{16,17,58-61} and label-free.^{19,62-66}

In relation to breast cancer, SERS has been used in several studies to distinguish cancerous and noncancerous cells,⁶⁷⁻⁷⁰ while only a few recent studies have examined the classification potential for EVs. One study successfully classified MCF-10A (nontumorigenic breast epithelium) and MDA-MB-231 (triple negative breast cancer) EVs using a nata de coco-based silver nanoparticle hybrid material following standard cell culture with EV-depleted serum and subsequent density gradient purification.¹⁴ However, while density gradient purification is a well-established and highly effective method for removing non-EV contaminants, the depletion of exogenous bovine EVs from serum supplements using ultracentrifugation is known to be only partially effective,⁷¹ and the remaining bovine EVs may convolute the acquired EV spectra.

In another study, EVs from MCF-7 (ER+/PR+), MDA-MB-231, and MCF-10A cells grown in commercially available EV-depleted serum and isolated by repeated ultracentrifugation were successfully classified by gold colloid SERS.⁷² However, protein and other co-precipitated contaminants are known to persist even after repeated ultracentrifugation steps with a great cost to total EV yield,⁷³ again potentially confounding the acquired spectra. In addition, no study has yet obtained spectra nor classified breast cancer EVs from HER2-positive cells, an important subtype used in clinical breast cancer scenarios.

Raman spectroscopy has recently shown promise for Extracellular vesicle (EV) characterization, particularly when paired with nanostructured plasmonic surfaces.⁷⁴ The plasmonic surfaces effectively amplify the normally weak EV Raman signal by several orders of magnitudes through the generation of strong nanoscale electric fields, making it possible to biochemically fingerprint EVs, at times down to the single EV level.⁷⁵ Following the application of machine learning algorithms, the acquired spectra may then be used to classify different subpopulations of EVs.⁷⁶

As EVs consist of non-chromophore biomolecules, they naturally have a very low SERS signal⁷⁷ and furthermore due to the relatively large size of EVs (30-150+nm) and the small active SERS distance of plasmonic particles (usually less than 5 nm), the use of plasmonic nanoparticles and unlabelled EVs is problematic and unlikely to generate strong SERS signals.⁷⁸ Labelled SERS can be used to selectively isolate and characterise specific subtypes of EVs from complex mixtures, but the labels can inhibit the access of EVs to the plasmonic surface and the obtained spectra may lack valuable information about the biomolecular contents of the captured EVs. Thus, efficient identification and characterisation of EVs at low concentrations using SERS is still an ongoing challenge.

As discussed above, the use of SERS to classify EVs has been primarily used in cancer applications^{62,76} but has recently begun to expand into other EV-related research fields.²⁹ However, for EV SERS to become a routine research or clinical characterization method, cost-effective, and versatile plasmonic surfaces with high sensitivity must be available. These surfaces should be easy to manufacture, stable over time, and highly reproducible from batch to batch.

It is an object of the present invention to go at least some way to overcoming or ameliorating any one or more of the above-mentioned disadvantages, and/or to at least provide the public and/or industry with a useful choice.

### SUMMARY OF INVENTION

In a first broad aspect, the present invention provides a method of manufacturing a surface-enhanced Raman spectroscopy (SERS) device comprising the steps of (a) depositing a Raman signal enhancing material as a substrate on a base layer, (b) using a pulsed laser source with a pulse width of less than one picosecond to pattern the surface of the substrate generating a patterned area.

In some embodiments, patterning the surface of the substrate comprises making repeated scans over the substrate with the laser source, with each scan being spatially separate so as to enlarge the patterned area to a desired size and to obtain a substantially homogeneously patterned substrate.

In some embodiments, the repeated scans result in scanned lines on the substrate, wherein there is separation between the scanned lines and the method further comprises adjustment of the separation between the scanned lines to match an effective beam waist generated by the laser source.

In some embodiments, the laser source is a femtosecond laser.

In some embodiments the repeated scans are made at a scanning speed ranging from about 0.5 to about 1.5 mm/s.

In some embodiments, the separation between the scanned lines is between about 0.5 to about 2 times the effective beam waist generated by the laser source, for example from about 0.5, 0.6, 0.7, 0.8, 0.9, 1, 1.1, 1.2, 1.3, 1.4, 1.5, 1.6, 1.7, 1.8, 1.9 or 2 times the spot size of the laser, and suitable ranges may be selected from any of these values, for example the separation may be between about 0.5 to about 2, about 0.5 to about 1.5, about 0.5 to about 1, about 0.7 to about 2, about 0.7 to about 1.5, about 0.7 to about 1, or about 1 to about 1.5 times the effective beam waist generated by the laser source, preferably between about 0.5 to about 2 times the effective beam waist generated by the laser source.

In some embodiments, the fluence incident on the substrate ranges from about 0.05 J/cm² to about 0.5 J/cm², for example from about 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5 J/cm², and suitable ranges may be selected from any of these values, for example from about 0.05 to about 0.5, about 0.05 to about 0.45, about 0.05 to about 0.4, about 0.05 to about 0.35, about 0.05 to about 0.3, about 0.05 to about 0.25, about 0.05 to about 0.2, about 0.05 to about 0.15, about 0.05 to about 0.1, about 0.1 to about 0.5, about 0.1 to about 0.45, about 0.1 to about 0.4, about 0.1 to about 0.35, about 0.1 to about 0.3, about 0.1 to about 0.25, about 0.1 to about 0.2, about 0.1 to about 0.15, about 0.2 to about 0.5, about 0.2 to about 0.45, about 0.2 to about 0.4, about 0.2 to about 0.35, about 0.2 to about 0.3, about 0.3 to about 0.5, about 0.3 to about 0.45, about 0.3 to about 0.4, about 0.3 to about 0.35, or about 0.4 to about 0.5, preferably from about 0.05 J/cm² to about 0.5 J/cm².

In some embodiments, the laser in step b) has a scanning speed ranging from about 0.5 to about 1.5 mm/s, for example from about 0.5, 0.6, 0.7, 0.8, 0.9, 1, 1.1, 1.2, 1.3, 1.4, or 1.5 mm/s, and suitable ranges may be selected from any of these values, for example 0.5 to 1.5, 0.7 to 1.5, 0.9 to 1.5, 1 to 1.5, 1.2 to 1.5, 1.4 to 1.5, 0.5 to 1.4, 0.7 to 1.4, 0.9 to 1.4, 1 to 1.4, 1.2 to 1.4, 0.5 to 1.2, 0.5 to 1, 0.5 to 0.8, 0.5 to 0.7, 0.7 to 1.5, 0.7 to 1.2, 0.7 to 1, 0.7 to 0.9, or 0.9 to 1, preferably from about 0.5 to about 1.5 mm/s.

In some embodiments, the fluence incident on the substrate is about 0.2 J/cm² and the repeated scans are made at a scanning speed of about 1.125 mm/s.

In some embodiments, the separation between the scanned lines is between about 0.5 to about 20 μm, for example about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 μm, and suitable ranges may be selected from any of these values, for example from about 0.5 to about 20, about 0.5 to about 10, about 0.5 to about 5, about 0.5 to about 2.5 about 1 to about 20, about 1 to about 10, about 1 to about 5, about 1 to about 2.5, preferably about 2.5 μm.

In some embodiments, the Raman signal enhancing material is deposited by sputter coating or thermal evaporation.

In some embodiments, the laser in step b) generates about 50 femtosecond (fs) to 1 picosecond (ps) pulses, for example about 50, 100, 110, 120, 130, 140, 150, 200, 250, 300, 350, 400, 450, 500, 550, 600, 650, 700, 750, 800, 850, 900, 950 fs or 1 picosecond pulses, and suitable ranges may be selected from any of these values, for example about 50 fs to about 1 ps, about 50 fs to about 750 fs, about 50 fs to about 500 fs, about 50 fs to about 250 fs, about 50 fs to about 200 fs, about 50 fs to about 150 fs, about 50 fs to about 140 fs, about 100 fs to about 1 ps, about 100 fs to about 750 fs, about 100 fs to about 500 fs, about 100 fs to about 250 fs, about 100 fs to about 200 fs, about 100 fs to about 150 fs, about 100 fs to about 140 fs, 200 fs to about 1 ps, about 200 fs to about 750 fs, about 200 fs to about 500 fs, about 200 fs to about 250 fs, 300 fs to about 1 ps, about 300 fs to about 750 fs, about 300 fs to about 500 fs, preferably about 200 fs, more preferably about 140 fs.

In various embodiments the laser has a pulse repetition rate between about 1 KHz and 1 mHz.

In various embodiments the laser generates 140 femtosecond (fs) pulses at a central wavelength of about 800 nm and a pulse repetition rate of about 1 kHz.

In some embodiments, the base layer comprises or consists of a material selected from the group consisting of glass, chromium, silicon, sapphire, silica and germanium.

In some embodiments, the base layer is a dielectric material with a surface roughness of less than about 10 nm.

In a second broad aspect, the present invention provides a surface-enhanced Raman spectroscopy (SERS) device, comprising a base layer and a substrate comprising a Raman signal-enhancing material disposed on the base layer, wherein a surface of the substrate comprises a plurality of features of positive and negative curvature.

In some preferred embodiments of the second aspect the curvature is in the range [−1,1] μm⁻¹.

In some embodiments, the SERS device comprises a plurality of features of positive and negative curvature with values that vary randomly across the substrate.

In some embodiments, the SERS device further comprises a plurality of nanoparticles. Preferably these are nanoparticles of a Raman signal-enhancing material.

In some embodiments, the plurality of nanoparticles is distributed randomly on

the surface of the substrate.

In some embodiments, the Raman signal-enhancing material comprises or consists essentially of gold, or comprises or consists essentially of silver. In some preferred embodiments the Raman signal-enhancing material layer comprises or consists essentially of gold. Alternatively, the Raman signal-enhancing material layer may be other appropriate materials known in the art.

In some embodiments the base layer comprises or consists of a material selected from the group consisting of chromium, glass, silicon, sapphire, silica and germanium.

In some embodiments the base layer comprises or consists of a material selected from the group consisting of silicon, sapphire, silica and germanium.

In some embodiments the base layer is a dielectric material with a surface roughness of less than about 10 nm, for example less than about 9 nm, 8 nm, 7 nm, 6 nm, 5 nm, 4 nm, 3 nm, 2 nm or 1 nm, and suitable ranges may be selected from any of these values, for example from about 1 to about 10, about 1 to about 9, about 1 to about 8, about 1 to about 7, about 1 to about 6, about 1 to about 5, about 1 to about 4, about 1 to about 3, about 1 to about 2, about 2 to about 10, about 2 to about 8, about 2 to about 6, about 2 to about 4, about 3 to about 10, about 3 to about 8, about 3 to about 6, about 3 to about 5, about 4 to about 10, about 4 to about 8, about 4 to about 6, about 5 to about 10, about 6 to about 10, about 6 to about 8, about 7 to about 10, or about 8 to about 10 nm.

In some embodiments of the first or second aspect, said Raman signal-enhancing material forms a layer on said base layer at least 200 nm thick, for example from about 200, 210, 220, 230, 240, 250, 260, 270, 280, 290 or 300 nm thick or greater, and suitable ranges may be for example from about 200 to about 10000, about 200 to about 8000, about 200 to about 5000, about 200 to about 3000, about 200 to about 2000, about 200 to about 1000, about 200 to about 500, about 200 to about 450, about 200 to about 400, about 200 to about 350, about 200 to about 250, about 250 to about 450, or about 300 to about 400 nm thick.

In a third broad aspect, the present invention provides a surface-enhanced Raman spectroscopy (SERS) device, comprising a base layer and a substrate comprising a Raman signal-enhancing material disposed on the base layer, wherein a surface of the substrate comprises a plurality of depressions.

In some embodiments, the base layer is a plastics layer, preferably a layer of silicone.

In some embodiments, Raman signal-enhancing material comprises or consists essentially of gold which has been deposited on the base layer using a two-step sputtering process.

Preferably said depressions are cup or bowl shaped.

Preferably said depressions are substantially uniformly spread over the device.

In some embodiments said depressions are micron sized, and can example have a size in the range of about 0.3 μm to 10 μm. For example, in some embodiments, the depressions can have a size of about 300 nm, or about 400, 500, 600, 700, 800, 900, 1000, 1100, 1200, 1300, 1400, 1500, 1600, 1800 or 2000 nm. In some embodiments, the depressions in said base layer are formed using nanobeads having a size in the range of about 400 nm to 1600 nm, for example about 500 nm to 1500 nm, about 600 nm to 1400 nm, about 700 nm to 1300 nm, about 800 nm to 1200 nm, about 900 nm to 1200 nm, about 900 nm to 1100 nm, or about 1000 nm. In some embodiments, the depressions in said base layer are formed using nanobeads of 1 μm diameter.

In some embodiments said plastics layer is between 1 mm and 1 cm thick. In some embodiments said plastics layer is approximately 1 mm thick.

In some embodiments comprising a plastics layer, said Raman signal-enhancing material forms a layer on said plastics layer at least 12.5 nm thick but may be between 30 and 110 nm thick, for example from about 35, 40, 45, 50, 55, 60, 65, 70, 75, 80, 85, 90, 95, 100, or 105 nm thick, and suitable ranges may be selected from any of these values, for example from about 30 to about 100, about 30 to about 90, about 30 to about 80, about 30 to about 70, about 30 to about 60, about 30 to about 50, about 40 to about 100, about 40 to about 90, about 40 to about 80, about 40 to about 70, about 40 to about 60, about 40 to about 50, about 50 to about 100, about 50 to about 90, about 50 to about 80, about 50 to about 70, or about 50 to about 60 nm thick. In some preferred embodiments the gold layer is approximately 50 nm thick.

In a fourth broad aspect, the present invention provides a surface-enhanced Raman spectroscopy (SERS) device, comprising a base layer and a substrate comprising a Raman signal-enhancing material disposed on the base layer, wherein a surface of the substrate comprises a plurality of protrusions.

In some embodiments, the base layer is a plastics layer, preferably a layer of polystyrene.

In some embodiments, Raman signal-enhancing material comprises or consists essentially of gold which has been deposited on the base layer using a two-step sputtering process.

Preferably said protrusions are dome shaped.

Preferably said protrusions are substantially uniformly spread over the device.

In some embodiments said protrusions are micron sized, and can example have a size in the range of about 0.3 μm to 10 μm. For example, the protrusions can have a size of about 300 nm, or about 400, 500, 600, 700, 800, 900, 1000, 1100, 1200, 1300, 1400, 1500, 1600, 1800 or 2000 nm. In some embodiments, the protrusions in said base layer are formed using nanobeads having a size in the range of about 400 nm to 1600 nm, for example about 500 nm to 1500 nm, about 600 nm to 1400 nm, about 700 nm to 1300 nm, about 800 nm to 1200 nm, about 900 nm to 1200 nm, about 900 nm to 1100 nm, or about 1000 nm. In some embodiments, the protrusions in said base layer are formed using nanobeads of 1 μm diameter.

In some embodiments said plastics layer is between 1 mm and 1 cm thick. In some embodiments said plastics layer is approximately 1 mm thick.

In some embodiments comprising a plastics layer, said Raman signal-enhancing material forms a layer on said plastics layer at least 12.5 nm thick but may be between 30 and 110 nm thick, for example from about 35, 40, 45, 50, 55, 60, 65, 70, 75, 80, 85, 90, 95, 100, or 105 nm thick, and suitable ranges may be selected from any of these values, for example from about 30 to about 100, about 30 to about 90, about 30 to about 80, about 30 to about 70, about 30 to about 60, about 30 to about 50, about 40 to about 100, about 40 to about 90, about 40 to about 80, about 40 to about 70, about 40 to about 60, about 40 to about 50, about 50 to about 100, about 50 to about 90, about 50 to about 80, about 50 to about 70, or about 50 to about 60 nm thick. In some preferred embodiments the gold layer is approximately 50 nm thick.

In a fifth broad aspect, the present invention provides a method of manufacturing a SERS device comprising the steps of: (a) applying a suspension of nanobeads substantially uniformly onto a flat support, (b) drying the suspension and support to form a layer of nanobeads on said support, (c) forming a polymer layer on said layer of nanobeads, (d) removing said polymer layer from said support and removing any nanobeads from said polymer layer, (e) depositing a Raman signal-enhancing material on said polymer layer.

In a sixth broad aspect, the present invention provides a method of manufacturing a SERS device comprising the steps of: (a) applying a suspension of nanobeads substantially uniformly onto a flat support, (b) drying the suspension and support to form a layer of nanobeads on said support, (c) forming a first polymer layer on said layer of nanobeads, (d) removing said first polymer layer from said support and removing any nanobeads from said first polymer layer, (e) forming a second polymer layer on said first polymer layer, (f) separating said second polymer layer from said first polymer layer, and (g) depositing a Raman signal-enhancing material on said second polymer layer.

In some embodiments of the fifth or sixth aspect, step (c) comprises applying a mixture of a polymer and a curing agent over said layer of nanobeads, and curing said mixture.

In some embodiments of the sixth aspect, step (e) comprises applying a layer of said second polymer to a flat support and heating the support; applying a layer of said second polymer to said first polymer layer and heating said second polymer layer; pressing the surface of said second polymer layer against the surface of said second polymer on said first polymer layer. In some embodiments, a hot plate is used to heat the support and first polymer layer. The temperature of the hot plate can in some embodiments be above 100° C., for example 120, 130, 140, 150, 160, 170, 180, 190 or 200° C. In some embodiments, said pressing is for a duration of less than 60 seconds, for example 50, 40, 30, 20 or 10 seconds. In some embodiments, said second polymer is applied to said first polymer layer by spin-coating.

In some embodiments of the fifth or sixth aspect, said flat support is a glass slide, and the method comprises steps of washing said glass slide to remove residue, and treating said glass slide to make it hydrophilic, prior to applying said suspension of nanobeads to said slide.

In some embodiments of the fifth or sixth aspect, the method further comprises a step of etching said Raman signal-enhancing material using an oxygen plasma treatment. This can have the benefit of further increasing the nanoscale surface curvature and the Raman signal enhancement.

In some embodiments of the fifth or sixth aspect, the Raman signal-enhancing material comprises or consists essentially of gold or silver, preferably gold.

In some embodiments of the fifth or sixth aspect, the nanobeads are between 400 nm and 2000 nm in size, preferably 1 μm in size.

In some preferred embodiments of the fifth or sixth aspect, said support or glass slide is washed with acetone followed by a separate step of washing with isopropyl alcohol.

In some preferred embodiments of the fifth or sixth aspect, the support or glass slide is treated to increase its hydrophilicity, for example using an oxygen plasma treatment.

Preferably said oxygen plasma treatment is radio frequency reactive ion etching (for example, (50W 50% Oxygen inlet 300s, Nordson March CS-1701).

Preferably said nanobeads are polystyrene beads of 1 μm in size.

In some preferred embodiments, a suspension of polystyrene beads is diluted to a ratio of 5% solid in pure water. Optionally the suspension of polystyrene beads can be sonicated, for example for one hour.

Preferably the drying of the suspension and support is in a controlled environment, preferably at 25° C. and 60% humidity, while the support is placed on a surface with 10 degree slope from horizontal. This results in large monolayer areas of nanobeads with sizes around a few millimetres square.

Preferably the drying of the suspension and support includes an additional step of putting the support in an oven at around 65° C. to completely dry the support or glass slide and increase the adhesion of beads to the surface of the support or glass slide.

Preferably the polymer used in step (c) is a silicone, for example is PDMS Sylgard 184 (Dow, US) mixed with a curing agent. Preferably the silicone is degassed before pouring on the support or glass slide.

Preferably the step of removing the polymer layer from the support or glass slide includes removing any nanobeads from the polymer layer. This may be done for example by placing the polymer layer in an acetone sonication bath, preferably for 15 minutes, but any appropriate time to remove the beads could be used.

Preferably after any nanobeads have been removed from the polymer layer in step (d) (for example by acetone sonication of the polymer layer), a polymer layer comprising silicone may be baked in an oven for 24 hours at a high temperature, preferably 100° C. This may improve the silicone surface for later coating.

In some embodiments of the fifth or sixth aspect, the Raman signal-enhancing material is gold, deposited using direct current sputtering.

Preferably the method of the fifth or sixth aspect uses a two-step gold sputtering process that creates a fractal structure observable in SEM images, that further enhances the electric field to provide higher efficiencies than are present for prior art substrates.

In some embodiments of the fifth or sixth aspect, the deposition of the gold is at a rate of about 0.5 nm per minute to provide a gold layer about 10-15 nm thick, then the rate is increased to about 6 nm per minute until the gold layer is about 30-100 nm thick.

In a seventh broad aspect, the present invention provides a method for identifying or classifying EVs in a sample, the method comprising the steps of (a) applying a sample comprising EVs to a SERS substrate, (b) obtaining one or more Raman spectra for each EV sample, (c) analysing the Raman spectra to identify or classify the EVs.

In some embodiments of the seventh aspect, prepared EVs are applied to a SERS device according to the second, third or fourth aspect, or a device prepared according to the first, fifth or sixth aspect.

In some embodiments of the seventh aspect, the Raman spectra are obtained using an excitation wavelength of 785 nm.

In some embodiments of the seventh aspect, the EVs are identified or classified using one or more of: principal component analysis (PCA); and a neural network, preferably using a neural network.

Due to relatively large sizes of even small EVs (30-150 nm) compared to chemical species, and EVs' lack of strong chromophore molecules, on a flat SERS substrate their SERS spectra are much weaker than those of chemical dyes like R6G. Their size also prevents them from fitting perfectly into the nanometric hotspots of a flat SERS substrate, in theory resulting in larger EVs producing weaker signals compared to the smaller EVs. Thus, the SERS substrates of the invention have larger hotspot areas suitable for EV SERS measurements, in contrast to flat gold surfaces, which provide weak to no Raman spectra for EVs.

In some embodiments, the Raman spectra are obtained using a laser source having an excitation wavelength of 532 nm or 785 nm, preferably 785 nm. It is believed that the 785 nm wavelength a 100-fold increase in the size of the SERS hotspot, compared to the 532 nm wavelength. This can provide a better Raman signal enhancement for larger molecules and EVs on the surface of the substrate, as they can be better fit within the hotspot areas.

In an eighth broad aspect, the present invention provides an in vitro method of diagnosing and/or monitoring the progression of a bacterial infection, viral infection, cancer or pre-eclampsia, the method comprising the identification and/or classification of extracellular vesicles in a sample by analysis of one or more SERS spectra of the sample.

In some embodiments of the eighth aspect, the SERS spectra have been obtained using the SERS device according to the first aspect, or a device prepared according to the second, third or fourth aspect.

In some embodiments of the eighth aspect, the method comprises (a) providing a sample comprising extracellular vesicles, (b) contacting the sample with the SERS device according to the first aspect, or a SERS device prepared according to the method of the second, third or fourth aspect, (c) obtaining one or more Raman spectra of the sample, (d) analysing the one or more Raman spectra using machine learning to identify and/or classify extracellular vesicles in the sample, and (e) determining the presence and/or progression of a bacterial infection, viral infection, cancer or pre-eclampsia.

In some embodiments the sample comprises viral, bacterial, cancer and/or placental extracellular vesicles.

In some embodiments the extracellular vesicles are placental extracellular vesicles, and the method is adapted to distinguish between healthy and pre-eclamptic samples by identifying and/or classifying extracellular vesicles in the sample.

In some embodiments the method is adapted to distinguish between samples from subjects with early onset pre-eclampsia and late onset pre-eclampsia.

In some embodiments the method allows for the rapid classification of breast cancer subtypes, bacteria or viruses.

In some embodiments the method can be used as a tool to diagnose and/or monitor breast cancer or other diseases.

In some embodiments the device and method can be used to detect and classify bacteria or viruses.

In some embodiments the device and method may provide information to enable diagnosis of a disease, or enable monitoring of the progress of a disease within a human or non-human body.

In some embodiments of the seventh or eighth aspect, the machine learning classifier has been trained according to the methods outlined herein.

In a further aspect, the present invention provides a SERS device according to the second, third or fourth aspect, or a device prepared according to the first, fifth or sixth aspect, for use in an in vitro method of diagnosing and/or monitoring the progression of a bacterial infection, viral infection, cancer or pre-eclampsia.

In a further aspect, the present invention provides a SERS device according to the second, third or fourth aspect, or a device prepared according to the first, fifth or sixth aspect, when used in an in vitro method of diagnosing and/or monitoring the progression of a bacterial infection, viral infection, cancer or pre-eclampsia.

In a further aspect, the present invention provides a kit for analysing extracellular vesicles (EVs) in a sample, the kit comprising a SERS device according to the second, third or fourth aspect, or a device prepared according to the first, fifth or sixth aspect, and machine learning software that can compare SERS spectra resulting from use of the device to a database or training data to classify and identify the spectra.

In a further aspect, the present invention provides a system to identify and/or classify extracellular vesicles (EVs) in a sample, preferably an in vitro sample, the system comprising the SERS device according to the second, third or fourth aspect, or a device prepared according to the first, fifth or sixth aspect, and machine learning software that compares spectra resulting from use of the device and compares that spectra to a database or training data to classify and identify the spectra.

### Machine Learning

In various embodiments in any one or more of the above aspects the machine learning algorithm or software is selected from the group consisting of deep convolutional neural networks, bottle neck classifiers, linear discriminant analysis (LDA), support vector machine (SVM), Random Forest (RF), Gaussian Process Classifier (GPC) and k-nearest neighbours (KNN). In various embodiments the machine learning algorithm reduces the dimension of the input Raman Spectra. In various embodiments the machine learning algorithm reduces the dimension linearly. In various embodiments the machine learning algorithm is a neural network classifier, in particular a bottleneck classifier. The various embodiments below may be applied to any of the machine learning aspects.

Some embodiments of the invention apply manifold machine learning algorithms, including t-SNE and UMAP, to EV SERS analysis. These algorithms are very selective for dimension reduction of all the obtained SERS spectra.

In a further aspect, the present invention provides a method of training a classifier for identification and/or classification of extracellular vesicles, the method comprising the steps of:
Obtaining input data comprising a plurality of Raman spectra; and
Training a neural network on the input data, the neural network comprising at least one bottleneck layer.

In some embodiments the bottleneck layer has a number of nodes and/or a dimension of one. In some embodiments the bottleneck layer has a number of nodes and/or a dimension of two. In some embodiments the bottleneck layer has a number of nodes and/or a dimension substantially equal to the output labels or required information. In some embodiments the bottleneck layer has a dimension of number of nodes substantially less than the preceding layer or layers, or the input layer. The bottleneck layer may have less than 20% the dimension of the preceding layer or input layer, less than 10% or less than 1%.

In some embodiments the neural network comprises at least one encoder portion preceding the bottleneck layer and at least one decoder portion following the bottleneck layer. The encoder portion preferably comprises one or more layers, each layer preferably comprising linear nodes. The decoder portion preferably comprises one or more layers, at least one layer preferably comprising non-linear nodes. The decoder portion may comprise an output layer configured to represent a label of the EV. The output layer may comprise softmax nodes. The output layer may have a dimension of two.

In a further aspect, the present invention provides a neural network which reduces the dimension of the data, preferably to one, such as a hybrid autoencoder-inspired neural network encourages the network to effectively separate the extracellular vesicles based on their specific labels in the latent (hidden or unmeasurable) layers of the neural network.

In a further aspect, the present invention provides a method of training a classifier for identification and/or classification of extracellular vesicles, the method comprising the steps of:
Obtaining input data comprising a plurality of Raman spectra;
Training a neural network on the input data, the neural network comprising a plurality of linear layers configured to reduce the dimension of the input data.

The use of a plurality of linear layers when reducing the dimension of the input data means that corresponding values of the spectra can be calculated from the values in the linear layer. For example, by calculating a dot product of the spectra and the obtained direction and adding a bias or offset. The input data can be obtained by measurement or provided by an external source.

In various embodiments the neural network comprises at least one dense layer with linear activation the dense layers arranged before the at least one layer in which the dimension of the data is one. In various embodiments the layer in which the dimension of the data is one is obtained by linear dimension reduction and/or a linear transformation. In various embodiments the neural network compresses the input data into the at least one layer in which the dimension of the data is one.

In various embodiments the neural network comprises at least one nonlinear activated dense layer, the nonlinear activated dense layer arranged after the at least one layer in which the dimension of the data is one.

In various embodiments the method may be configured to classify two or more extracellular vesicles. In various embodiments the extracellular vesicles any one or more of: normotensive extracellular vesicles and preeclampsia extracellular vesicles. In various embodiments the Raman spectra are surface enhanced Raman spectra.

In a further aspect, the present invention provides a method to identify and/or classify extracellular vesicles, the method comprising the steps of:
Obtaining a trained classifier, the trained classifier comprising the classifier of the above aspects;
Applying the trained classifier to one or more Raman Spectra of extracellular vesicles; and
Receiving a classification result for the one or more Raman Spectra from the trained classifier.

In a further aspect, the present invention provides a method of training a classifier for identification and/or classification of extracellular vesicles, the method comprising the steps of:
Obtaining a plurality of Raman spectra; and
Applying a bottleneck classifier to the plurality of Raman spectra.

A bottleneck classifier comprises at least one layer that contains relatively few nodes compared to the previous layer or layers. These are used in autoencoders to reduce dimensionality. Optionally the bottleneck classifier has at least one layer with a number of nodes equal or substantially equal to the independent parameters. Optionally this layer is the lowest dimension layer in the network.

In a further aspect, the present invention provides a method of training a classifier for identification and/or classification of extracellular vesicles, the method comprising the steps of:
Obtaining input data comprising a plurality of Raman spectra;
Training a machine learning algorithm on the input data.

In various embodiments the machine learning software or algorithm is selected from, or any one or more of, the group consisting of neural networks, classifiers, deep convolutional neural networks, bottle neck classifiers, linear discriminant analysis (LDA), support vector machine (SVM), Random Forest (RF), Gaussian Process Classifier (GPC) and k-nearest neighbours (KNN).

In a further aspect, the present invention provides a method of training a classifier for identification and/or classification of extracellular vesicles, the method comprising the steps of:
Obtaining input data comprising a plurality of Raman spectra;
Training a neural network on the input data, the neural network comprising an auto-encoder with a bottleneck layer.

In a further aspect, the present invention provides a method of training a classifier for identification and/or classification of extracellular vesicles, the method comprising the steps of:
Obtaining input data comprising a plurality of Raman spectra;
Training a neural network on the input data, the neural network comprising an input layer configured to receive an input spectra, an output layer configured to output a reconstructed spectra and a bottleneck layer between the input layer and the output layer.

In some embodiments the neural network comprises a second output layer, the second output layer configured output spectral labels. The second output layer may be connected to a regression network or layers. The output layers preferably have relative output weights or training weights, preferably the reconstructed spectra outlet has a greater weight than the spectral labels. Optionally the weight is 10, 100 or 1000 times greater than the spectral label. Optionally the training is at least partially supervised. Optionally the input data represents a range or dilution range of a mixture of particles. Optionally the particles are EVs.

In some embodiments the portion of the network before the bottleneck is an encoder and the portion of the network after the bottleneck is a decoder. Optionally the encoder comprises linear nodes.

In some embodiments the bottleneck layer has a number of nodes and/or a dimension of one. In some embodiments the bottleneck layer has a number of nodes and/or a dimension of two. In some embodiments the bottleneck layer has a number of nodes and/or a dimension substantially equal to the output labels or required information. In some embodiments the bottleneck layer has a dimension of number of nodes substantially less than the preceding layer or layers, or the input layer. The bottleneck layer may have less than 20% the dimension of the preceding layer or input layer, less than 10% or less than 1%.

In some embodiments the classifiers are applied to mixtures of two or more populations of Raman Spectra. In embodiments the plurality of Raman spectra comprises a dilution series or represent a range of mixture ratios of two or more populations of Raman Spectra. Optionally, the Raman spectra represent any one of EVs, lipoproteins, or other biomolecules in solution. In some embodiments the method is configured to distinguish a mixture ratio of a mixture of particles having a SERS response.

The disclosed subject matter also provides a method or system which may broadly be said to consist in the parts, elements and features referred to or indicated in this specification, individually or collectively, in any or all combinations of two or more of those parts, elements or features. Where specific integers are mentioned in this specification which have known equivalents in the art to which the invention relates, such known equivalents are deemed to be incorporated in the specification.

Further aspects of the invention, which should be considered in all its novel aspects, will become apparent from the following description.

### DRAWING DESCRIPTION

A number of embodiments of the invention will now be described by way of example with reference to the drawings as follows.

FIG. 1 (a) shows a graph of the εϕϕ and μϕϕ distribution in R-Z plane and (b) shows the curvature that can mimic the material distribution in (a).

FIG. 2 shows a spherical nanoparticle on (a) flat and (b) wrapped substrate and normalized electric field distributions around the nanoparticle in (c) flat and (d) wrapped substrate.

FIG. 3 shows a volumetric average (a) and (b) of normalized electric field in the designated hotspot in FIG. 2 (a) and (b) for the flat and wrapped substrate, respectively, and (c) the ratio of (b) to (a).

FIG. 4 shows the expected Raman enhancement by the wrapped substrate, (b) the ratio of expected Raman enhancement of wrapped to flat substrate, and (c) the effect of the radius of the curvature in Raman enhancement.

FIG. 5 shows the distribution of normalized electric field around the cubic nanoparticle in the (a) flat and (b) wrapped substrates.

FIG. 6 shows a nanobead and soft lithography fabrication schematic and SEM images showing steps of (a) nanobead deposition on a flat support, (b) addition of a polymer layer to the deposited nanobeads, (c) removal of the cured polymer layer, (d) cleaning the cured polymer layer of nanobeads, (e) the cured polymer layer after cleaning, (f) deposition of gold on the cured polymer layer; the SEM image shows 300 nm nanobeads trapped on the gold layer to represent EVs, (g) size distribution of the gold nanoparticles which form the top of the gold layer using two step gold deposition (scale bar=2 μm).

FIG. 7 shows Nanocup SERS substrate characterisation showing (a) SEM of 300 nm beads trapped in the nanocup SERS hot spots (scale bar=4 μm), (b) optical microscopy image of tightly packed nanocup structures across the entire surface (scale bars=5 μm), and (c) AFM of the nanocup SERS demonstrating the desired structure.

FIG. 8 shows a nanobead and soft lithography fabrication schematic for forming a substrate of Example 1A with steps of (1) nanobead deposition on a flat support, (2) addition of a polymer layer to the deposited nanobeads, (3) curing of the polymer layer, (4) removal of the cured polymer layer, (5) inversion of the cured polymer to provide a stamp, (6) applying a second polymer to the stamp, (7) curing of the second polymer, (8) deposition of the second polymer onto a flat support, (9) removal of the stamp, (10) deposition of gold on the cured polymer layer, and photographic image of the substrate.

FIG. 9 shows SEM images of substrates prepared according to Example 1A which where subjected to oxygen plasma etching for a duration of 1, 2, 3, 4, 5, 6, and 7 minutes respectively (scale bars=2 μm).

FIG. 10 shows SERS spectra obtained using 10⁻⁵ M R6G solution on the substrates shown in FIG. 9 and a control substrate prepared according to Example 1A which had not been subjected to oxygen plasma etching; and a graph showing the relative SERS enhancement correlated to duration of oxygen plasma etching treatment.

FIG. 11 shows size exclusion chromatography purification of small EVs showing protein and particle amounts of each collected fraction for (A) MCF-7, (B) BT-474 and (C) BT-20, NTA size distributions and TEM images of pooled EV-rich fractions for (D) MCF-7, (E) BT-474 and (F) BT-20 small EVs (scale bars=500 nm), (G) and pooled particle and protein counts for all three samples from a single isolation.

FIG. 12 shows the obtained Raman spectra of Rhodamine b (Rb) with concentrations of (a) 10⁻⁴ M, (b) 10⁻⁵ M, and (c) 10⁻⁶ M using the curved SERS substrate, and (d) 10⁻⁵ M using flat SERS substrate.

FIG. 13 shows three replicates (a), (b) and (c) of BT-20 EV Raman spectra.

FIG. 14 shows the 2D PCA transformation of the normalized spectra of Rb in FIG. 12.

FIG. 15 shows the normalized Raman spectra of (a) BT-20 cell line with different (1×10¹⁰ and 1×10⁹ EVs/ml) concentrations, and (b) 2D PCA transformation of the BT-20 EV spectra with different concentrations.

FIG. 16 shows the normalized Raman spectra of (a) BT-20, (b) BT-474 and (c) MCF-7 EVs with their specified band for each type of biomolecule.

FIG. 17 shows the 2D PCA transformation of (a) EV spectra from BT-20, BT-474 and MCF-7 EVs with the concentration of 1×10¹⁰ EVs/ml, and transformation of Raman spectra of (b) BT-20 cell line EVs with the concentration of 1×10⁹ when it is transformed by the obtained transformation in (a).

FIG. 18 shows the probability distribution in 2D PCA plane obtained using CSV, KNN, LDA, RF, GPC and DT classifiers.

FIG. 19 shows EV characterisation in relation to Example 2 showing (a) size distributions of all bacterial EV samples from NTA, (b) representative TEM of UPEC-R-DG, Nissle-R-SEC, and K12-RF-SEC E. coli EVs (scale bars=200 nm), and (c) acquired SERS spectra of all E. coli EV samples after baseline establishment and denoising with mean, min/max, and standard deviation represented as red (line), yellow (outer shaded area), and cyan (inner shaded area), respectively.

FIG. 20 shows machine learning results for classification by strain between K12-RF-SEC and UPEC-RF-SEC showing (top) averaged EV SERS spectra, (middle) Linear Discriminant Analysis (LDA), Gaussian Process Classifier (GPC), K-nearest neighbour (KNN), and Support Vector Classifier (SVC), and (bottom) first and second PC scores.

FIG. 21 shows confusion matrices showing classification efficiency for all E. coli EVs using different machine learning methods, including (a) LDA, (b) GPC, (c) KNN, (d) SVC, (e) ANN and (f) RF.

FIG. 22 shows all sample classification following PCA transformation showing (a) cumulative explained variance vs the number of principal components, scores of (b) first, (c) second, and (d) 100th principal components, (e) SVC and (f) ANN accuracy when different number of principal components are considered.

FIG. 23 shows the result of the manifold machine learning approaches comparing (a) conventional PCA to (b) t-SNE and (c) UMAP for all E. coli EV SERS spectra.

FIG. 24 is a diagram of an alternative machine learning system of the present invention showing convolution and identity blocks in the CNN system of the present invention.

FIG. 25 is a diagram the overall architecture of the alternative machine learning system, the CNN system of the present invention, that may be used for classification of biological samples.

FIG. 26 shows machine learning results for classification by bacteria strain between averaged SERS spectra for Nissle-R-SEC and UPEC-R-SEC showing (top) averaged EV SERS spectra, (middle) Linear Discriminant Analysis (LDA), Gaussian Process Classifier (GPC), K-nearest neighbour (KNN), and Support Vector Classifier (SVC), and (bottom) first and second PC scores.

FIGS. 27 and 28 show machine learning classification results for pairwise comparisons between plain and iron supplemented culture media.

FIGS. 29 and 30 show machine learning classification results for SEC and DG-purified UPEC EVs from both types of culture media.

FIG. 31 shows a Scanning Electron Microscope (SEM) image of gold thin films and resulting direct laser-induced nano structuring of thin films (LINST) patterns for (a,c) magnetron sputter coated and (b,d) thermal evaporated gold thin films (insets corresponding to higher magnification images of the same nanopatterned area, scale bars=1 μm) prepared as described herein. FIGS. 31a to 31d show the successful deposition of gold particles on the base layer. FIGS. 31e and 31f show Atomic Force Microscopy (AFM) images and profiles for (e) magnetron sputter coated and (f) thermal evaporated gold thin films show the surface relief in greater detail while the bottom figures show the depth profile across the sample along the dotted line. Features of positive and negative curvature can be clearly seen.

FIG. 32a shows COMSOL (a finite element modelling software) simulation results of LINST plasmonic surfaces are shown in (g) which is a schematic of the simulated geometry. FIGS. 32b-32e show (h) the hotspot areas for 785 nm for different substrate curvatures and particle radii, (i) shows the hotspot areas for 532 nm excitation for different substrate curvatures and particle radii and normalized electric field distributions for instances with maximum hotspot areas using (j) 785 nm excitation and (k) 532 nm excitation are also shown. FIGS. 32b-32e show that the substrate curvature strongly affects the resulting hotspot area and thus the electric field enhancement.

FIG. 33 shows the Raman spectra of R6G obtained at different concentration levels using the LINST SERS technique. The top curve (offset) shows the spectra for a concentration of 10⁻⁶ M, the middle a concentration of 10⁻⁷ M and the bottom curve a concentration of 10⁻⁸ M. All spectra were captured using a 532 nm laser and with a one second acquisition time. This show that signals can be detected at levels approaching nanomolar concentrations for short (one second) acquisition times.

FIGS. 34a-34f show LINST SERS using 10⁻⁷ M Rhodamine 6G (R6G) showing (a) optical image of the Raman imaging area used, (b) Raman image based on peak height at 1358cm−1, (c) corresponding amplitude of the first Principle Component (PC) of the euclidean normalized Raman image spectra, (d) PC score of (c), (e) distribution of 2 clusters obtained using K-mean clustering, and (f) center of the clusters with their corresponding colors in (e) where center 1 is marked 1 and center 2 is marked 2.

FIGS. 35a-35f show LINST SERS using 10⁻⁶ M R6G showing (a) optical image of the larger Raman imaging area used in (b) Raman image based on peak height at 1358cm⁻¹, (c) amplitude of the first PC, (d) PC score of (c), (e) distribution of 2 clusters obtained using K-mean clustering, and (f) center of the clusters with their corresponding colors in (e) where center 1 is marked 1 and center 2 is marked 2.

FIG. 36 (a) shows an optical image of the area used to obtain the LINST SERS using late onset pre-eclampsia (LOPE) EVs, (b) distribution of 5 K-mean clusters over the imaged area, (c) Raman spectra of each cluster in (b) where trace 5 shows no Raman signals and occurs outside the laser treated area, the remaining traces (1 to 4) show the SERS spectrum of the EVs with some minor differences in amplification of signal near 1150. These traces occur predominantly in the laser treated area but extend further due to the presence of re-deposited gold nanoparticles.

FIG. 37 shows classification of LINST SERS of normotensive and preeclamptic EVs where (a) shows the spatial distribution of the top K-mean clusters of each normotensive (NT), early onset pre-eclampsia (EOPE) and late onset pre-eclampsia (LOPE) samples showing that LINST SERS resulted in Raman spectra based on which normotensive (NT), EOPE and LOPE samples could be identified and classified as being NT/EOPE and LOPE. Below each map is shown the histogram of clusters. The histograms vary significantly between NT, EOPE and LOPE samples, while the variation with each type is less. FIG. 37(b) shows the spectrum of each cluster (offset vertically) depicted in (a) with their corresponding color where the trace corresponding to center 1 is marked 1, center 2 is marked 2, center 3 is marked 3, center 4 is marked 4, center 5 is marked 5 and center 6 is marked 6.

FIG. 38 Averaged placental EV data showing nanoparticle tracking analysis (NTA) size distributions, and normalized SERS spectra for (a-b) NT, (c-d) EOPE, and (e-f) LOPE placental EVs. Distributions of all obtained spectra in two dimensions are shown in (g) PCA, (h) t-SNE, and (i) UMAP planes. In figures (b), (d) and (f), band i) corresponds to protein/lipid, band ii) corresponds to phenylalanine, band iii) corresponds to DNA/protein, bands iv) correspond to protein, band v) corresponds to lipid, band vi) corresponds to DNA/protein/lipid, band vii) corresponds to DNA/RNA, and band viii) corresponds to lipid/phospholipids.

FIG. 39 Proposed hybrid autoencoder-inspired classifier showing (a) proposed network design, (b) projection of training and test set in the one dimensional latent space.

FIG. 40 shows calculated direction obtained by the linear activated layers of the network of FIG. 36. Band i) corresponds to protein/lipid, band ii) corresponds to phenylalanine, band iii) corresponds to DNA/protein, bands iv) correspond to protein, band v) corresponds to lipid, band vi) corresponds to DNA/protein/lipid, band vii) corresponds to DNA/RNA, and band viii) corresponds to lipid/phospholipids.

FIG. 41 is a comparison of the custom Deep Convolutional Neural Network (CNN) and Bottleneck Classifier (BC) algorithms with conventional machine learning classifiers including Linear Discriminant Analysis (LDA), Support Vector Machine (SVM), Random Forest (RF), Gaussian Process Classifier (GPC), and K-nearest Neighbor (KNN).

FIG. 42 Proposed network design for hybrid autoencoder bottleneck classifier configured to determine ratio of EVs in solution.

FIG. 43 Proposed network design for hybrid autoencoder bottleneck classifier configured to determine ratio of EVs in solution, with additional spectral label decoder.

FIG. 44 Linear node suitable for network of FIG. 42 or 43.

FIG. 45 Example SERS spectrum for EVs at different ratios.

FIG. 46 Test outputs after training of proposed networks of (a) FIG. 42 and (b) FIG. 43 on SERS ratios, and (c) example use of network of FIG. 42.

FIG. 47 Synthesized example of hybrid autoencoder bottleneck classifier in two dimensions showing (a) input, (b) latent space by height, (c) latent space by width and (d) projection vectors.

### DETAILED DESCRIPTION

Recently, some efforts have been made to either classify raw Raman spectra directly⁸⁰ or automatically denoise and correct their baselines⁸¹ using Convolutional Neural Network (CNN). Application of different variants of CNN architectures to classify raw Raman spectra of chemicals showed that the CNN can even lead to a better classification accuracy over the training on raw Raman signal than the condition in which the pre-processed data has been used for the training purposes. Some proposed CNN classifiers can outperform conventional classification methods including linear discriminant analysis (LDA), support vector machine (SVM), k-nearest neighbour (KNN), and artificial neural network (ANN).⁸⁰

The inventors have designed and fabricated surface-enhanced Raman spectroscopy (SERS) substrates (SERS devices) and tested their capabilities using purified breast cancer EVs of three different subtypes; EVs from bacteria; and EVs from tissue explant cultures of both normotensive and preeclamptic placentae. As viral particles have similar characteristics to disease particles or molecules or bacteria, it is anticipated that the substrates, system and methods may be used in the identification and classification of viruses. As such, the substrates and sensing system of the present invention are capable of identifying and classifying biological samples. The SERS substrates and corresponding analytics, such as machine learning, deep learning and artificial intelligence, together herein referred to as the “sensing system”, have the ability to effectively fingerprint and efficiently EVs of different types.

The inventors have designed a substrate or SERS device of the present invention using transformation optics to achieve extremely high electric fields around nanometric surface features. The main idea is that the gradient index material conjugated with the normal plasmonic waveguide can first increase the wave coupling from the incident laser into the plasmonic surface⁸² and second, the presence of the material distribution can help the propagated plasmonic wave confine near the material gradient so that it propagates for longer distances.⁸³ Both of these statements have been proven semi-analytically using coupled mode theory.⁸⁴ However, material gradients cannot be directly used around a SERS surface as it would prevent the investigated analyte from reaching the surface where the maximum electromagnetic wave confinement and resulting maximum Raman enhancement exist. Transformation optics can be applied to solve this problem by transforming the material gradient into a curvature in space without changing the behaviour of the electric field. This is the reverse process of the usual applications of transformation optics in which the physical effects of space curvature are implemented by material distribution.

In one preferred aspect of the invention the inventors have designed, fabricated and tested a space-curvature inspired SERS substrate composed of a tightly packed nanocup (or nanobowl) pattern with improved granularity on its gold surface. Both numerically and analytically, in combination, these geometric features significantly increase the SERS enhancement for chemical species and are particularly advantageous for label-free biological applications, such as EV characterization. This highly effective structure is also readily accessible compared to many other reported SERS substrates, requiring only nanoparticle and soft lithography followed by a two-step sputter deposition of gold.

In a related preferred aspect of the invention, the inventors have designed, fabricated and tested a SERS substrate composed of a tightly packed nanodome pattern. The nanoscale surface curvature/energy, and thus the potential SERS signal enhancement of substrate, has been further increased using etching techniques, like plasma etching. In addition to providing a completely different nanoscale topography, the process for forming the SERS substrate is also scalable, as it employs a nanoscale stamp which can be used repeatedly to make many replicates of the nanodome substrate.

Another preferred aspect of the invention provides a simple, tuneable, and scalable method of SERS device fabrication, termed laser-induced nano structuring of thin films (LINST). Substrates with the desired surface features can be prepared by nanopatterning directly on thin films of a Raman signal-enhancing material such as gold or silver, using a pulsed laser source where the pulse width is less than one picosecond, preferably using a femtosecond laser. The versatile SERS substrate is suitable for both chemical and EV analysis, as it produces both concave and convex curvature on the surface. The LINST process enables the soft nano structuring of thin films of a Raman signal-enhancing material such as gold or silver, avoiding any complete removal of the Raman signal-enhancing material layer while creating nanoscale roughness across the entire surface that enables straightforward SERS analysis of both chemical species and EVs. The inventors have found the LINST method described herein to provide a simple and fast way to prepare SERS substrates suitable for EVs, since there is no need to pattern a substrate prior to deposition of the Raman signal-enhancing material.

The inventors have also unexpectedly found that, as a by-product of the LINST laser machining process, nanoparticles of Raman signal-enhancing material of varying size are redeposited or distributed randomly across the surface of the substrate. Thus, in some embodiments of the invention, a plurality of nanoparticles are distributed randomly across the surface of the substrate. For SERS applications, these redeposited nanoparticles may produce even greater Raman signal enhancement than the base nanopattern itself. In some embodiments, the nanoparticles can be for example, in a size range of about 1-100 nm, for example about 20-80 nm, or about 20-60 nm. In some embodiments the nanoparticles can be as small as 5 nm or even smaller. This distinct additional feature cannot be obtained using LIPSS patterning of bulk materials. These nanoparticles have an enhancement effect on the SERS spectra of EVs in particular. As detailed herein, the nanoparticles produce moderate Raman enhancement for a small-molecule chemical standard, and produce significantly greater SERS enhancement for EVs.

As demonstrated below, direct laser-induced nano structuring of thin films (LINST) can produce scalable and reproducible SERS substrates for EV fingerprinting and classification. Direct laser machining of the gold thin films easily achieves the SERS amplification needed to characterize Raman spectra of EVs, and this SERS amplification is believed to be partially due to the redeposition of gold nanoparticle debris from the laser-machining process. Directly patterning SERS-active thin films can provide additional advantages in terms of fabrication scalability, and/or can improve Raman signal enhancement compared to some subsurface patterning techniques, since coating a base pattern with a Raman signal-enhancing material smooths the surface features in the base pattern, thus reducing the curvature.

In some embodiments, the present invention utilizes a combination of substrates, Raman spectroscopy, machine learning and the use of extracellular vesicles (EVs) to provide a powerful means to detect and identify a wide range of biological materials.

Combining a SERS substrate with the application of machine learning algorithms on the acquired spectra enables fingerprinting and classifying of biological samples, such as for cancers, or bacteria and viruses. This platform and characterization approach will enhance the viability of EVs and nano plasmonic sensing systems towards clinical utility for breast cancer, classification and identification of bacteria and viruses and many other applications to improve human health. The accuracy of various deep learning algorithms for classifying SERS EV spectra was also investigated and the results indicated that the custom approaches of deep learning algorithms, Deep CNN and BC, perform as well or better than other known approaches.

### Curvature

As used herein, the term “concave curvature” refers to a negative curvature and “convex” curvature refers to a positive curvature. Depressions in a SERS substrate have a negative curvature and protrusions have a positive curvature.

In some embodiments of the invention where the substrate has both positive and negative curvature, the curvature can, for example, be in a range of [−1,1] μm⁻¹, and can include values selected randomly from within this range, for example from values of about −1.0, −0.9, −0.8, −0.7, −0.6, −0.5, −0.4, −0.3, −0.2, −0.1, 0, 0.1, 0.2, 0.3, 0.4, 0.5, 0.6, 0.7, 0.8, or about 0.9 μm⁻¹. The inventors have found this curvature range particularly suitable for the size of EVs investigated.

In some embodiments of the invention where the substrate has a plurality of nanoparticles distributed across the surface, the nanoparticles are smaller and therefore have higher curvature. The nanoparticles may typically be tens of nanometres in size, or can be as small as 5 nm or even smaller, therefore having a curvature as high as 200 μm⁻¹ or even higher.

In some embodiments of the invention where the depressions or protrusions are formed using nanobeads, the curvature results from the size of the nanobeads used to form them. By way of example only, a method using a nanobead of diameter 1 μm (radius 0.5 μm) can produce a depression which, once coated with gold is about 0.47 um in depth (i.e. radius 0.47 μm). Curvature being the inverse of radius of curvature, the depression therefore has an absolute value of curvature of 2.13 μm⁻¹. Nanobeads can be formed in a wide range of sizes, and thus the radius of curvature for the substrates of the invention has a corresponding range. For example, a nanobead of size 25 nm would have a curve radius of 12.5 nm and an absolute value of curvature of about 80 μm⁻¹. Sizes of the nanobeads are discussed further below.

### EVs

Extracellular vesicles (EVs) is a collective term covering various subtypes of cell-released, membranous structures, called exosomes, microvesicles, microparticles, ectosomes, oncosomes, apoptotic bodies, and many other names. EVs have a wide range of properties depending on their biogenesis. The International Society for Extracellular Vesicles (ISEV) has published guidelines on minimal information for studies of extracellular vesicles (MISEV) including nomenclature relating to the physical characteristic of size.⁸⁵ For instance, small EVs can be described as <100 nm or <200 nm [small], or >200 nm [large and/or medium]). Consistent with this nomenclature, as used herein, “EVs” refers to EVs of any size and “small EVs” refers to EVs smaller than 200 nm diameter, for example 50-200 nm diameter or 30-150 nm diameter.

In some embodiments of the invention, the use of CELLine adherent bioreactors allows the production of vast numbers of EVs compared to conventional cultures, without the risk of contamination with exogenous bovine serum EVs, while size exclusion chromatography provides a simple and effective method for removing contaminating proteins.

The potential utility of the devices of the invention in biomedical applications was investigated, by classifying various types of EVs including three distinct subtypes of breast cancer EVs, three distinct subtypes of bacterial EVs, and three distinct types of EVs of placental origin, by applying several machine learning algorithms to the acquired spectra. It will be appreciated that the demonstrated utility indicates the invention is applicable to analyse and classify EVs from other sources, including viruses.

### Breast Cancer EVs

In some embodiments of the invention, the EVs originate from breast cancer cells. To demonstrate these embodiments of the invention, breast cancer cell lines were cultured in bioreactors (CELLine AD 1000) enabling the isolation of copious amounts of EVs that are cultured in physiologically relevant conditions, and which can be rapidly purified from contaminating proteins using automated size exclusion chromatography, as discussed herein. Due to the incredibly high yields, the minimal dependence of the Raman spectra on EV concentration over several orders of magnitude was demonstrated. In addition, clear differences in the averaged Raman spectra allowed the rapid classification of breast cancer subtypes using machine learning algorithms.

### Bacterial EVs

In some embodiments of the invention, the EVs are of bacterial origin. The rapid growth pace of bacteria allows cells to release EVs in quick response to changes in environmental cues, resulting in EV molecular compositions that may represent specific temporal or environmental conditions.⁸⁶ EVs from bacteria can be selectively characterised using the SERS substrate of the present invention and the acquired spectra can be used for classification purposes. Several parameters appear to influence the SERS spectra of E. coli EVs including strain, purification method, and culture medium, which can be classified using machine learning approaches. Collectively, these findings establish the incredible sensitivity and potential utility of SERS for bacterial EV analysis, as classification-enabling differences were seen in each sample subtype, despite the fact that all of the samples tested were from the same species of bacteria. Thus, using SERS to characterise bacterial EVs provides a powerful and sensitive tool to detect or classify their parental bacteria cells' identity or condition. Hence, the substrate and sensing system of the present invention may be used to identify and classify bacteria. cl Placental EVs

In some embodiments of the invention, the EVs are of placental origin. EVs were cultured and isolated from tissue explant cultures of both normotensive and preeclamptic placentae, and using the device according to the invention for SERS analysis, were found to produce classifiably distinct spectra following the application of machine learning algorithms. This demonstrates placental EVs provide abundant and accessible liquid biopsy markers for conditions such as preeclampsia.

### SERS Device

As used herein, the term “SERS device” refers to an arrangement of a base layer, with a Raman signal-enhancing material layer (or substrate) disposed on the base layer. So as not to inhibit the access of EVs to the plasmonic surface or to obscure information in the obtained spectra relating to the biomolecular contents of the EVs, the substrate of the SERS device of the invention is preferably “label-free”. Label-free substrates are free of probes that could target specific antigens on the EV surface, for example antibodies, aptamers, or peptides.

### Base Layer

In some embodiments the base layer can comprise or consist of a plastics material, also referred to herein as a polymer, including a silicone such as polydimethylsiloxane (PDMS), or a polymeric organic compound such as PMMA (polymethylmethacrylates) or polystyrene. In other embodiments the base layer can comprise or consist of a material selected from the group consisting of chromium, glass, silicon, sapphire, silica and germanium.

### Raman Signal-Enhancing Material

In some preferred embodiments the Raman signal-enhancing material layer is gold. Alternatively, the Raman signal-enhancing material layer may be silver, or other appropriate materials known in the art. As mentioned above, the Raman signal-enhancing material is also known as a substrate. The inventors believe use of a gold substrate in the SERS devices of the invention can result in a 100-fold increase in the size of the SERS hotspot. A silver substrate can oxidize more quickly than gold and hence the surface degrades faster, leading to changes in the plasmonic properties over time which can complicate analyses.

### Nanobeads

In some embodiments of the invention, the SERS device is fabricated using nanobeads. The term “nanobeads” is used herein to refer to polymer beads. Nanobeads can be formed from polymers including, but not limited to: polystyrene; polyacrylnitrile; melamine; and sulfate latex. Nanobeads suitable for use in the invention can have a wide range of sizes, including a size in the range of about 25 nm to 10000 nm, for example about 30, 40, 50, 60, 70, 80, 90, 100, 120, 140, 160, 180, 200, 220, 220, 240, 260, 280, 300, 320, 340, 360, 380, 400, 420, 440, 460, 480, 500, 550, 600, 650, 700, 750, 800, 850, 900, 950, 1000, 1050, 1100, 1250, 1300, 1350, 1400, 1450, 1500, 1600, 1700, 1800, 1900, 2000, 2200, 2400, 2600, 2800, 3000, 3200, 3400, 3600, 3800, 4000, 4500, 5000, 5500, 6000, 6500, 7000, 7500, 8000, 8500, 9000, 9500 or 10000 nm. Suitable ranges for the nanobead size may be selected from any of these values, for example from about 25 to about 8000, about 30 to about 7000, about 40 to about 600, about 50 to about 5000, about 60 to about 4000, about 70 to about 3500, about 80 to about 3000, about 90 to about 2500, about 100 to about 2000, about 25 to about 2000, about 30 to about 1800, about 30 to about 1700, about 30 to about 1600, about 30 to about 1500, about 40 to about 1400, about 50 to about 1350, about 60 to about 1300, about 70 to about 1250, about 80 to about 1200, about 40 to about 2000, about 60 to about 1800, about 80 to about 1700, about 100 to about 1600, about 150 to about 1500, about 200 to about 1400, about 300 to about 1350, about 400 to about 1300, about 500 to about 1250, about 600 to about 1200, or about 800 to about 1200 nm.

### Polymer

In some embodiments of the invention, the SERS device is fabricated using a polymer. The polymer can be selected from a liquid prepolymer of a corresponding polymeric organic compound, for example PMMA (polymethylmethacrylates), or a polymeric organosilicon compound (silicones). During fabrication of the SERS device the polymer can be cured as known in the art, for example using a curing agent or heat. A preferred polymer is a silicone such as PDMS (Sylgard 184, Dow) or Ecoflex™ (Smooth-On, Inc., US), typically used with a curing agent.

In some embodiments of the invention, the SERS device is fabricated using a first polymer and a second polymer. Where the first polymer is preferably a polymeric organic compound or polymeric organosilicon compound as discussed in the paragraph above, the second polymer can be a different polymer which can mould against the first polymer without adhering to the first polymer, for example an organic polymer or copolymer suitable for replica molding, such as polystyrene or polycaprolactone. A preferred second polymer is 10% polystyrene in anisole.

### Support

In some embodiments of the invention, the SERS device is fabricated on a support. The term “support” is used herein to refer to a flat structure which can receive a suspension of nanobeads, and is robust enough to be oven-dried. In some embodiments, the support is hydrophilic, or can be treated to increase its hyrdrophilicity. In an embodiment, the support is a glass slide.

### Chemical Standard; Chemical Testing

The sensitivity and reproducibility of SERS substrates and devices useful for the methods described herein can be tested using appropriate chemical standards. Chemical standards include chromophores which exhibit a high Raman scattering cross section and well-characterized Raman spectra. Chemical standards mentioned herein include Rhodamine 6G, and Rhodamine B (RhB); the person skilled in the art will understand other chemical standards can be used, including but not limited to adenine, Rhodamine 123, RBITC, MGITC, crystal violet, and 4-MBA.

Throughout the description like reference numerals will be used to refer to like features in different embodiments.

Unless the context clearly requires otherwise, throughout this specification, the words “comprise”, “comprising”, and the like, are to be construed in an inclusive sense as opposed to an exclusive or exhaustive sense, that is to say, in the sense of “including, but not limited to”.

As used herein “(s)” following a noun means the plural and/or singular forms of the noun.

As used herein the term “and/or” means “and” or “or” or both.

As used herein the term “in vitro” with reference to a method means a method carried out outside of the body of an organism, preferably and animal, more preferably a human. Similarly, “in vitro” with regards to a sample refers to a sample that is outside of the body of an organism, preferably and animal, more preferably a human.

It is intended that references to a range of numbers herein also incorporate references to all rational numbers within that range (for example, a reference to a range of 1 to 5 also incorporates, for example 1, 1.3, 2, 2.1, 3, 3.5, 4, 4.4 and 5) and also any range of rational numbers within that range (for example, 1 to 5, 1.3 to 4.5 and 2.6 to 3.2). On this basis, all sub-ranges of all ranges disclosed herein are considered expressly disclosed. These examples serve only to demonstrate what is specifically intended. As such, any and all possible combinations of numerical values between the lowest and the highest value mentioned herein are therefore considered be expressly covered by the disclosure herein.

### EXAMPLES

### Materials and Methods

### Thin Film Deposition

The gold films coated on the silicone layer of Example 1 and Example 1A below were applied using direct current sputtering (also referred to herein as Magnetron sputter coating or sputter coating) with a 99.99% pure gold target, in a two-step coating process, first at a rate of 0.5 nm per minute until a 12.5 nm thickness was achieved then the deposition rate was increased to 6 nm per minute (Q150R S, Quorum). This results in a gold layer that is at least 12.5 nm thick but may be up to 110 nm thick.

Two methods of gold thin film deposition were used to prepare films for LINST patterning (Example 4). For the first method, Magnetron sputter coating with a Q150R sputter coater (Quorum) was used in order to deposit a 200 nm layer of pure gold on the surface of Silicon wafers (p-doped [111]). The process was run with a current of 50 mA which resulted in deposition rate of 6 nm per minute. For the second method, a thermal evaporator was used to firstly deposit 10 nm of chromium followed by 200 nm of gold. Other suitable deposition methods may also be used in accordance with the invention. Examples of such suitable deposition methods will be apparent to a person skilled in the art.

### Laser-Induced Nanostructuring of SERS-Active Thin Films (LINST)

Femtosecond laser surface patterning was conducted on the deposited gold thin films from step a. above. The laser setup consisted of a Ti:Sapphire laser system combining an ultrafast oscillator (VI-TARA) with a LEGEND regenerative amplifier (Coherent). This system generates 140 femtosecond (fs) pulses at a central wavelength of 800 nm and a maximum pulse repetition rate of 1 kHz which was maintained constant in all examples. At 1 KHz, the laser system generated up to 1.5 mJ pulse energy. Beam polarization and power were controlled with a waveplate, a variable attenuator and neutral density filters. Suitable waveplates, variable attenuators and neutral density filters for use in this method will be apparent to a person skilled in the art. The 2 mm laser beam was focused on to the gold surfaces by a 5X Mitutoyo objective with a numerical aperture of 0.14 to achieve a theoretical focused beam waist of 11.2 μm.

In order to slowly nanostructure the surface and avoid the complete ablation of the SERS-active thin films, the power was carefully tuned and optimized to test the effect of fluences ranging from 0.05 J/cm² to 0.6 J/cm². Also, scanning speeds (velocities) ranging from 0.5 to 1.5 mm/s were tested. Laser polarization was set parallel to the scanning direction. The use of these fluences led to an effective spot size of approximately 2.5 μm (measured from scanning electron microscope (SEM) images) accounting for a very small part of the focused Gaussian beam above the ablation threshold of the material. It is also important to note that the diameter of a laser machined structure's width depends on the material's ablation threshold and the nonlinear response, which typically yields an interaction area smaller than the beam waist. Samples were moved using a three-axis motorized stage at different scanning speeds with a position accuracy of 1 μm while the whole process was monitored using an auxiliary Charge Coupled Device (CCD) camera mounted in a parallel arm.

Following the laser nanopatterning, the resulting samples were briefly cleaned with compressed air and kept in a clean atmosphere to prevent any contamination present prior to the SERS measurements. While scanning speeds of between 0.5 to 1.5 mm/s were used in this example, any appropriate scanning speed could be used, however, the scanning speed must consistent to ensure that the same number of pulses per millimeter is incident across the sample and provide a uniform surface.

The tested parameters described above resulted in different ablated micro and nanoscale patterns. For fluences higher than 0.5 J/cm², all the tested scanning speeds resulted in complete ablation of the gold thin film having the thickness tested, in certain areas.

Repeated scans were made over the substrate with the laser source, with each scan being spatially separate so as to enlarge the patterned area to the desired size and to obtain a substantially homogenously patterned substrate.

Optionally, the method above may include a step in which there is an adjustment of the separation between the scanned lines to match effective beam waist. This aims to improve or optimise the homogeneity and continuity in the LINST process.

A SEM image (not shown) of a LINST pattern on thermally evaporated gold fabricated with a fluence of 0.57 J/cm² and a scanning speed of 1.125 mm/s, showed the gold thin film was completely ablated at some spots. When the fluence was further increased or the speed decreased, these ablation spots became bigger in number and size leading to a weaker SERS effect. Similarly, a SEM image (not shown) of a LINST pattern on magnetron sputtered gold fabricated with a fluence of 0.2 J/cm² and 1.125 mm/s and with a line spacing of 5 μm did not generate a continuously patterned SERS substrate. Thus, if the spacing of the scanned lines is not matched to the line width resulting from the choice of fluence and scanning speed, the LINST patterning will have limited continuity leading to a relative decrease in the SERS effect.

Therefore, for smaller fluences, leading to soft LINST (i.e. gold thin-film ablation smaller than thin-film thickness), the effective spot size and line width were characterized via SEM, and 1×1.5 mm machined areas were fabricated by adjusting the line spacing according to the measured effective spot size and line width. The device fabricated with a fluence of 0.2 J/cm², scanning speed of 1.125 mm/s, and a separation between the scanned lines of 2.5 μm showed the best SERS signal enhancement for deposited gold thin films prepared by both the Magnetron sputter coating and thermal evaporation thin film deposition methods described above for the films for LINST patterning. This was established using a Raman chemical test with Rhodamine 6G (R6G) dye. As discussed herein, other dyes could be used, particularly those that have well characterised Raman spectra.

Subsequently, these parameters were used to create a number of identical LINST devices, which were used for the chemical and EV SERS testing discussed in Examples 4-6 below.

### LINST Surface Characterization

The surface morphology and topography of the LINST samples prepared as described above were visualized using SEM (Hitachi SU-70) and Atomic Force Microscopy (AFM) (Cypher-ES AFM from Asylum Instruments) with a standard ‘TAP-150AIG’ cantilever. The AFM scans were performed with a drive frequency of 141.7 kHz and 3V free amplitude which corresponds to a height difference of approximately 100 nm. All the images were taken in repulsive mode with a set point of 2V.

### Bioreactor Culture and Breast Cancer EV Isolation

EVs from three different cell lines representing different subtypes on the breast cancer spectrum were taken, including MCF-7 (ER+/PR+/HER2−), BT-474 (ER+/PR+/HER2+) and BT-20 (triple negative),⁸⁷ and cultured in CELLine AD 1000 bioreactor flasks as previously described.⁸⁸

Briefly, cells were seeded in the cell chamber in DMEM (Gibco) with 10% FBS (Merck) and 1% Pen/Strep (Gibco) and gradually adapted to Advanced DMEM/F-12 (Gibco) with 2% CDM-HD (Fibercell), 1% Glutamax (Gibco), and 1% Pen/Strep (Gibco) over the course of 4 weeks.

To isolate EVs, the 15 ml of conditioned media from the cell chamber of the bioreactor was centrifuged at 2,000×g for 10 min to remove cells and other debris. The supernatant was then centrifuged at 10,000×g for 30 min (JA.30-50 Ti rotor, Avanti, Beckman Coulter) to pellet the large EVs (also known as microvesicles). This supernatant was then ultracentrifuged at 100,000×g for 70 min (JA.30-50 Ti rotor, Avanti, Beckman Coulter) to yield a crude small EV pellet (also known as nanovesicles). This pellet was resuspended in 700 μl PBS and stored at −80° C. until needed.

500 μl of the crude small EV suspension was purified by loading it onto a 35 nm qEV original size exclusion chromatography column (SEC, Izon) and fractions 7 through 26 were collected using an automated fraction collector (Izon, 500 μl per fraction). High-Sensitivity BCA assay (Pierce, ThermoFisher Scientific) was performed for each of the collected fractions to determine their protein concentration.

### Breast Cancer EV Characterization

SEC-purified small EV fractions were diluted in Phosphate buffered saline (PBS) at a 1:100 ratio and measured with a NS300 Nanosight (Malvern Panalytical). Three 30 second videos were taken under low flow conditions (Screen gain:2, Camera level:8) and characterized using the Nanosight 3.4 software (Screen gain:10, Detection threshold:7) to calculate mean and mode particle diameters, concentration, and size distributions of each fraction. These results, combined with protein amounts from the BCA assay were used to determine the EV-rich, protein-contaminant poor fractions, which were then pooled for SERS and transmission electron microscopy (TEM). These pooled fractions were also then characterized using NTA and BCA.

Prior to TEM or SERS, EV samples were transferred from a PBS buffer to ultrapure water by loading 500 μl of the purified small EVs into a Vivaspin 500 (Sartorius) centrifugal concentrator with a 100 kDa cutoff and spun at 10,000×g until most of the PBS had flowed through the filter (roughly 10 min). 450 μl of ultrapure water was then added to the filter and the centrifugation process was repeated twice more before finally suspending the EVs in 100 μl ultrapure water. This preparation step is important for both TEM and SERS to reduce the presence of salt crystals following dehydration of the sample.

Negative staining TEM of purified small EVs was conducted by adsorption onto Formvar-coated copper grids (Electron Microscopy Sciences) for 5 min. Excess liquid was removed with filter paper (Whatman) and the copper grid was then transferred to 20 μL of 2% uranyl acetate for 2 min. Excess liquid was again removed with filter paper and the grid was allowed to dry under a lamp for 10 min. Grids were visualized on Tecnai G2 Spirit TWIN (FEI, Hillsboro, OR, USA) transmission electron microscope (TEM) at 120 kV accelerating voltage. Images were captured using a Morada digital camera (SIS GmbH, Munster, Germany).

### Bacterial Culture and EV Isolation

Three Escherichia coli strains were investigated; Uropathogenic E. coli (UPEC) strain 536 (O6:K15:H31),⁸⁹ probiotic Nissle 1917,⁹⁰ and laboratory model strain MG1655 (K-12, ATCC® 47076).⁹¹ Culture and EV isolation methods have been previously published in detail,⁸⁶ the contents of which are herein included by reference. Briefly, bacterial cells were grown in either of two iron conditions: iron restricted, in plain RPMI 1640 medium (R) (Thermo Fisher Scientific) or iron sufficient, in RPMI medium supplemented with 10 μM iron(III) chloride (RF) to better reflect physiological conditions. At the desired incubation time, bacterial cells were removed from the culture by centrifugation and filtration. Cell-free EV-containing supernatants were concentrated to smaller volumes with 100 kDa Vivaflow 200 cassettes (Sartorius AG) and EVs were pelleted by ultracentrifugation at 75,000×g for 2.5 h at 4° C. (Beckman Avanti J-301), then resuspended in PBS. EVs were then further purified with either of two well established purification methods: Density Gradient Centrifugation (DG) using an iodixanol (Optiprep, SigmaAldrich) gradient or Size Exclusion Chromatography (SEC) using a qEV Original column (70 nm, Izon Science).⁸⁶ Hereinafter, EV samples are referred to using the notation “Strain-Culture Medium-Purification Method”. For example, EVs from uropathogenic E. coli (UPEC) cells grown with iron supplementation and purified with size exclusion chromatography are referred to as “UPEC-RF-SEC”.

### Bacterial EV Characterisation

EV-rich fractions from both SEC and DG purification methods were determined by protein (Pierce™ BCA Protein Assay, ThermoFisher) and particle quantification using nanoparticle tracking analysis (NTA) using an NS300 Nanosight (Malvern Panalytical), then pooled for analysis. Once EV-rich fractions were pooled, they were diluted at a 1:250 ratio in PBS and three 30 second videos were taken under low flow conditions (Screen gain:2, Camera level:14) and characterised using the Nanosight 3.4 software (Screen gain:10, Detection threshold:6) to calculate mean and mode particle diameters, concentration, and size distributions. Prior to TEM or SERS, EV samples were transferred from PBS buffer to ultrapure water by loading 200 μl of the purified EVs into a Vivaspin 500 (Sartorius AG) centrifugal concentrator with a 100 kDa cuttof and centrifuging at 10,000×g until most of the PBS had flowed through the filter (roughly 10 minutes). Ultrapure water (ThermoFisher), 450 μl, was then added to the filter and the centrifugation process was repeated twice more before finally suspending the EVs in 100 μl ultrapure water. Negative staining TEM of purified EVs was conducted by adsorption onto Formvar coated copper grids (Electron Microscopy Sciences) for 10 minutes. Excess liquid was removed with filter paper (Whatman) and the copper grid was then transferred to 20 μL of 2% filtered uranyl acetate for 2 minutes. Excess liquid was again removed with filter paper and the grid was allowed to dry under a lamp for 10 minutes. Grids were visualised on Tecnai G2 Spirit TWIN (FEI, Hillsboro, OR, USA) transmission electron microscope (TEM) at 120 kV accelerating voltage. Images were captured using a Morada digital camera (SIS GmbH, Munster, Germany).

EV Isolation from Placentae

Chorionic villi of term placenta were excised into small pieces of approximately 400 mg each for culture and EV isolation. Each piece was incubated overnight in a Netwell™ insert in a Falcon® 12-well plate filled with Advanced DMEM/F-12 (Dulbecco's Modified Eagle Medium/Ham's F-12, Thermo Fisher Scientific). The media was supplemented with 2% FBS (fetal bovine serum, Life Technologies) and 1% Penicillin-Streptomycin (Life Technologies).

EVs were isolated from the conditioned culture media by differential centrifugation. First, the culture media was centrifuged at 2,000×g for 5 minutes to pellet any placental debris, cell components, and larger EVs. The resulting supernatant was centrifuged at 20,000×g for 1 hour at 4° C. (Sorvall WX 100+Ultracentrifuge, ThermoFisher) to pellet large EVs and the remaining supernatant was again centrifuged at 100,000×g for 1 hour at 4° C. to pellet small EVs. Any standard centrifuge can be used in the method described and suitable centrifuges will be apparent to a person skilled in the art, for example Sorvall WX 100+Ultracentrifuge, ThermoFisher. The resulting small EVs were resuspended in 500 μL of 1×phosphate buffered saline (PBS) to subsequently perform size exclusion chromatography (SEC) in a 35 nm qEV Original column (Izon) by initially collecting fractions 7-20. The EVs then underwent ultrafiltration for buffer exchange immediately prior to SERS as well as EV characterization as described in more detail below.

### Placental EV Characterization

EV-rich fractions (7-10) from SEC were initially determined by protein (Pierce™ BCA Protein Assay, ThermoFisher) and particle quantification using nanoparticle tracking analysis (NTA) using an NS300 Nanosight (Malvern Panalytical), then pooled for analysis. Pooled EVs were diluted at a 1:500 ratio in PBS and three 30 second videos were taken under low flow conditions (Screen gain:1, Camera level:14) and characterized using the Nanosight 3.4 software (Screen gain: 10, Detection threshold:6) to calculate mean and mode particle diameters, concentration, and size distributions.

### Raman Analysis

Prior to SERS, EV samples were transferred from PBS buffer to ultrapure water by loading 200 μl of the purified EVs into a Vivaspin 500 (Sartorius AG) centrifugal concentrator with a 100 kDa cutoff and centrifuging at 10,000×g until most of the PBS had flowed through the filter (roughly 10 minutes). 450 μl of ultrapure water was then added to the filter and the centrifugation process was repeated twice more before finally suspending the EVs in 100 μl ultrapure water.

For all SERS measurements, 1 μl of EVs (in an ultrapure water suspension) per square millimetre, with the concentration of ≈1×10¹⁰ EVs per millilitre, were dropped on the SERS surface and dried quickly in a 40° C. oven to avoid coffee ring effect and increase the homogeneity of the EVs' distribution on the SERS surface. Raman measurements were carried out using a Horiba LabRAM HR Evolution confocal Raman microscope by using 785 nm laser and 50×microscope objective. 50 SERS spectra were taken for each EV sample, with a minimum distance of one laser spot size between acquisition locations. The laser power at the surface of SERS was controlled using neutral density filter and set to 10 percent of the maximum power (100 mW). A 10 sec acquisition time for the detector was chosen per measurement. Then, the baseline was established and noise was removed automatically using previously established asymmetric least squares smoothing.⁹²

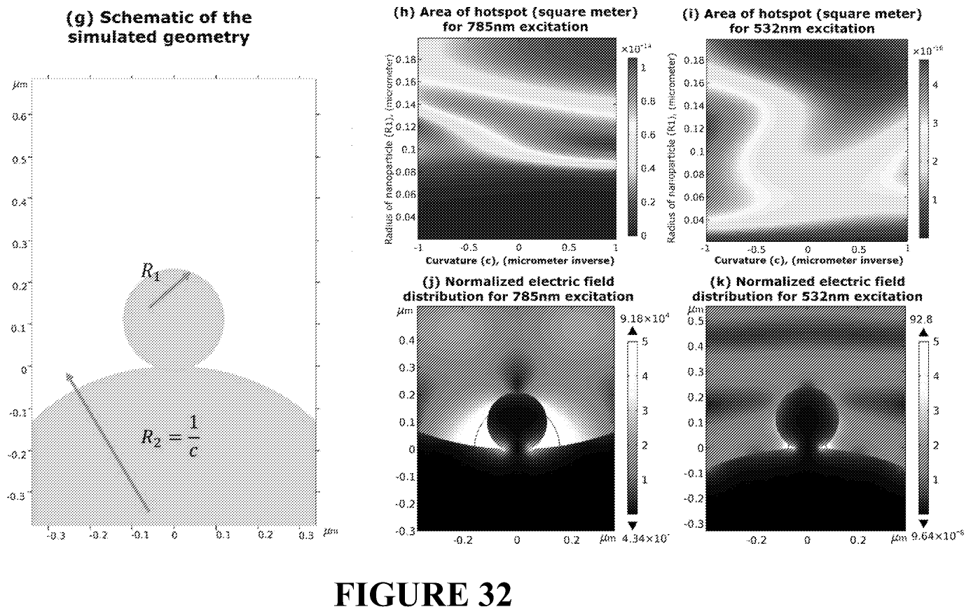

Raman spectra were acquired using a LabRAM HR Evolution confocal Raman microscope (Horiba) with either 532 nm or 785 nm excitation wavelengths. Using longer wavelengths is often preferable when dealing with biological samples since it reduces the amount of fluorescence and thus improves the signal to noise. 785 nm is also better when using gold as the substrate, as the optical loss at that wavelength is lower than at 532 nm. This is seen in FIG. 32 where the enhancement and hot-spot areas are better at 785 nm than 532 nm. Other materials as the substrate may be better utilised at shorter wavelengths. An example of such a material is silver.

Microscope objectives of 5× (NA=0.14), 10× (NA=0.26), and 50× (NA=0.42), where NA is numerical aperture, were used for the different purposes of this work. The laser power was controlled using a neutral density filter and set to 1% to 25% percent of the maximum power (100 mW) depending on the sample test. The percentage of maximum power was chosen in order firstly, to avoid damaging the EVs and secondly, to reduce the time taken for a measurement. A lower power could be used but at the expense of taking longer measurements. However, as long as the power is high enough to produce a signal that is all that is needed. All measurements were obtained with a 0.1 s to 10 s acquisition time, depending on the laser setup and choice of microscope object with only 1 accumulation. The system utilizes a 600 gr/mm blazed grating in conjunction with a notch filter to remove Rayleigh scattered light. Detection was conducted through a liquid nitrogen (−70° C.) CCD array (1024×256 pixels) detector. A confocal hole was set to 250 μm in back-scattering geometry. Then, the baseline was established and noise was removed using asymmetric least squares smoothing.⁹²

### Multivariate Analysis and Machine Learning

Machine learning and multivariate analyses including Principal Component Analysis (PCA) and t-distributed Stochastic Neighbour Embedding (t-SNE) transformations and K-mean clustering were carried out using the established Python library, Sklearn.⁹³ Machine learning and deep learning methods were implemented using TensorFlow python package.⁹⁴

### Substrate Design Method

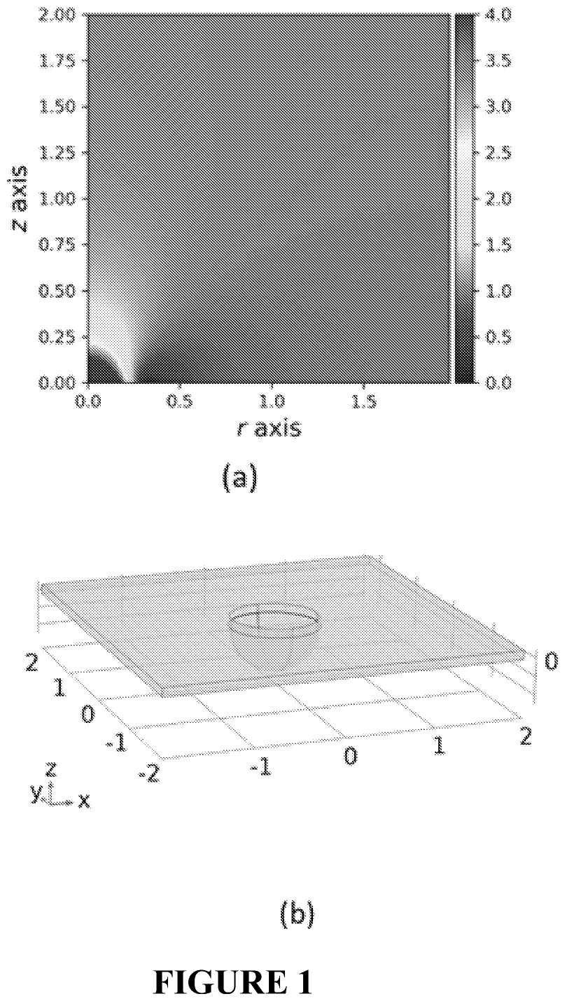

Consider a relative permittivity and permeability distribution of εφφ and μφφ as shown in FIG. 1(a). This material is placed on top of a flat metallic surface and also has rotational symmetry along the vertical z axis as illustrated in FIG. 1 (a). Theoretically, any plasmonic nanoparticle placed inside this area will create a resonant electric field with significantly higher amplitude than a bare particle. However, any high index material will prevent the analyte from reaching the surface of the particle where we have the maximum electric field and hence the highest Raman emission. We can apply the reverse method of transformation optics to turn this material gradient into a curved empty space so that particle is not surrounded by any material. As we have cylindrical symmetry, quasi-conformal transformation optics can be applied.⁹⁵ The detailed process of this reverse transformation was previously described in references 55,96, the contents of which are included herein by reference. This transformation changes the flat substrate into a surface with constant curvature as illustrated in FIG. 1(b). By placing a nanoparticle in the curved substrate, there is an added benefit of further amplification of the resonant field while the analyte can freely reach the nanoparticle surface.

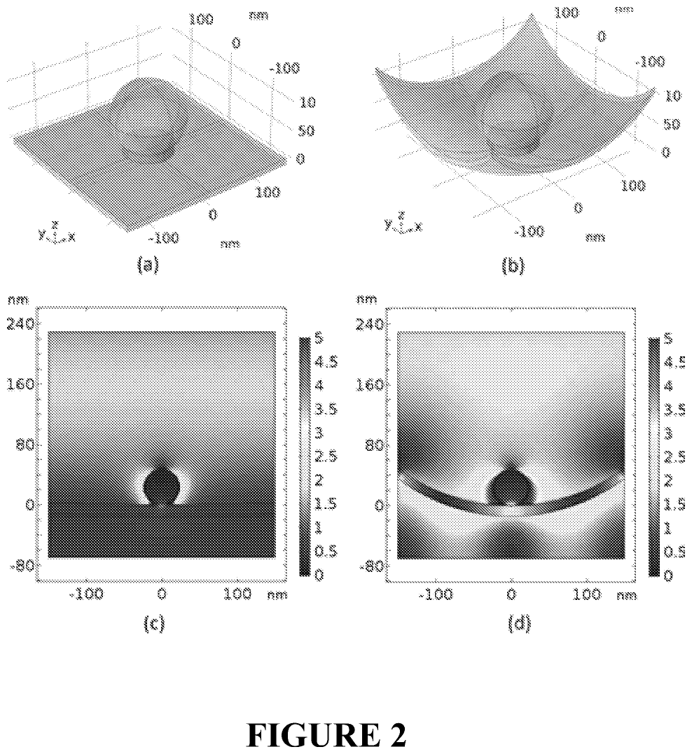

COMSOL Multiphysics™ simulation platform was used to model this concept by simulating different scenarios in which a gold nanoparticle with variable size is placed on either a flat or curved gold substrate as shown in FIGS. 2(a) and (b). This plasmonic structure was illuminated with 785 nm light from the top and we solved for the resulting electric field. The amplitude of the normalized electric fields relative to the incident field for the flat and curved substrate are shown in FIGS. 2(c) and (d), respectively. It can be seen that the electric field around the nanoparticle is enhanced in both cases while the size of the hot spot is clearly significantly bigger for the curved substrate. However, for a quantitative assessment, the volumetric average of the normalized electric field in the hotspot area (area which is marked by the blue cylinder at the contact point of spherical nanoparticle and substrate in FIGS. 2 (a) and (b)) is calculated.

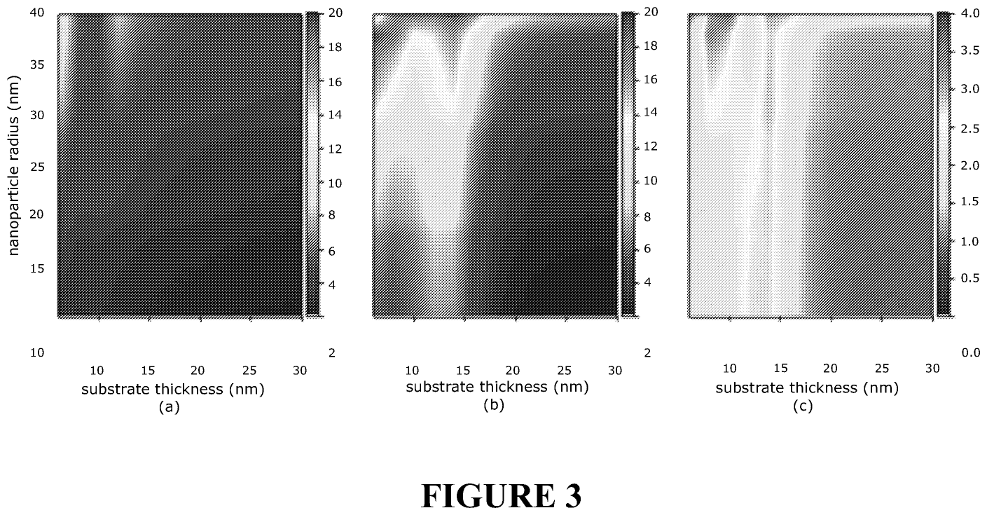

The result for the flat and curved substrates, when various thicknesses of the substrate and sizes of nanoparticles were considered, are shown in FIG. 3 (a) and (b), respectively. The electric field enhancement ratio is also demonstrated in FIG. 3 (c). As expected the average electric field is enhanced for almost all values for the thickness of substrate and the size of nanoparticle.

It should be noted that these simulations do not accurately model the small region where the ideal nanoparticle touches the substrate due to the minimum meshing size.⁷ This may cause some error in the results in this region but as this is a localized effect it does not significantly alter the overall results. Furthermore, such idealized shapes are impossible to fabricate and in fact the Finite Element Model (FEM) shape could be a more realistic approximation compared to our ideal model. This problem has been addressed analytically for structures smaller than 200 nm in references 7,97 and the inventors have also addressed this problem for the larger structures by introducing a simulation method.^{98,99}

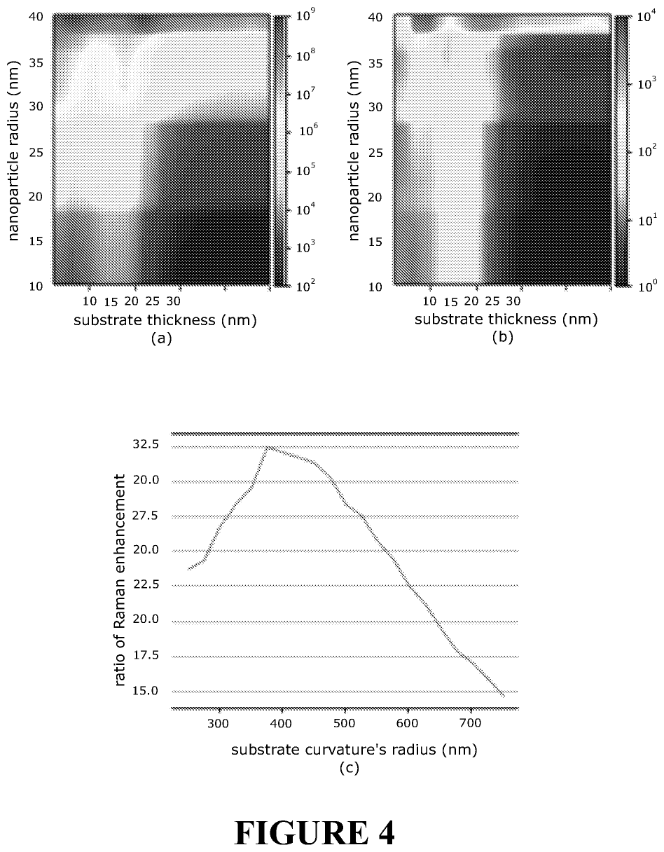

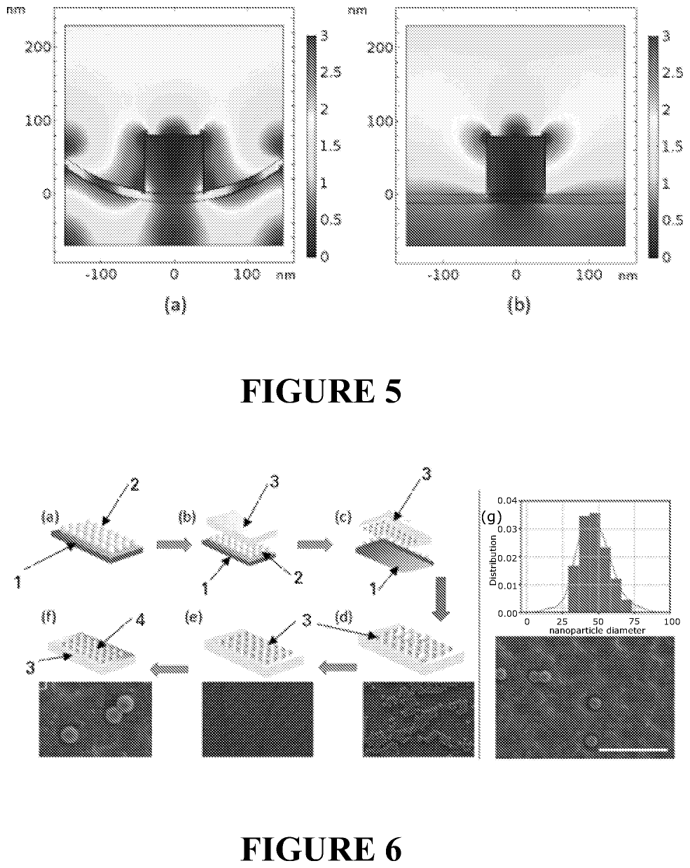

Knowing the expected electric field distribution allows the calculation of the expected Raman enhancement, which is proportional to the amplitude of the enhanced field near the plasmonic structures divided by the amplitude of the incident electric field, all to the fourth power.⁶ This expected enhancement is shown in FIG. 4(a) along with the improvement compared to the flat substrate in (b) for a range of different designs. The amount of enhancement for flat and curved substrate is found to be between 1 to 3 orders of magnitude higher for some nanoparticle sizes and the substrate thicknesses. To show the effect of the substrate radius of curvature on Raman enhancement, we also simulated the scenario in which the nanoparticle radius was chosen to be 20 nm with 1 nm substrate thickness and the radius of substrate curvature was varied between 250 nm to 750 nm. The calculated ratio of Raman enhancement between the flat and curved substrate for this scenario is shown in FIG. 4(c). To clearly demonstrate that this enhancement is due to the curvature of the substrate and not the curvature of the nanoparticle, we also simulated a cubic nanoparticle for both a flat and curved substrate, as is shown in FIG. 5 and again the curved substrate results in a significantly higher electric field.

### Example 1—Fabrication of a Substrate (SERS Device) by a Combination of Soft and Nanoparticle Lithography

A combination of soft and nanoparticle lithography were used to fabricate a warped substrate or SERS device. The process of manufacture for this example is shown in FIG. 6. First, a cleaned positively charged glass slide 1 is washed with acetone. Then followed by isopropyl alcohol to remove any residue from the glass surface. Then, the glass slide was oxygen plasma treated using radio frequency reactive ion etching (in this example, 50W 50% Oxygen inlet 300s, Nordson March CS-1701, however other appropriate etching methods may be used). This process adds OH⁻¹ to the surface of the glass and makes it hydrophilic.

A suspension of 1 μm polystyrene nanobeads (89904-5ML-F, SigmaAldrich) was diluted to the ratio of 5 percent solid in ultrapure MilliQ water. After sonicating the nanobeads for one hour to avoid any aggregation in suspension. A portion of the prepared bead suspension 2 (50 μl) is then dropped on the oxygen plasma cleaned glass slide 1 (see FIG. 6(a)). The glass slide was gently tilted until a uniform spread of suspension was achieved on the top surface. Then, the suspension was dried in a controlled environment, at 25° C. and 60% humidity, while the slide was placed on a surface with 10 degree slope from horizontal. This process is more efficient than Langmuir and spin coating to fabricate a large number of samples. Large areas of mono layer beads with sizes around few millimeter square, particularly in the middle of glass slides can be achieved using this method. The dried glass slide was then put in an oven at 65° C. to dry and increase the adhesion of beads to the glass surface. It will be appreciated that in other embodiments, other appropriate temperatures capable of resulting in an appropriate dryness of the glass slide may be used.

Next, a negative mould of the beads was created using an appropriate polymer, in this case a silicone, PDMS (Sylgard 184, Dow), mixed with a curing agent, at a 10:1 ratio. Referring to the numbering of the schematic in FIG. 6 which illustrates the general protocol, the silicone was degassed to remove bubbles, then poured onto a prepared glass slide 1 with a thickness of 1 mm, resulting in a thin silicone layer 3 on the bead layer 2 and degassed again. After being baked at 65° C. for 2 hours, the silicone layer 3 was gently peeled from the glass slide 1 with some beads typically remaining stuck to the silicone layer (see FIG. 6(d)). Any remaining beads were removed from the silicone layer using an acetone sonication bath, for a duration of 15 minutes. The resulting silicone is similar to that shown in FIG. 6(e), with a plurality of cup or bowl like depressions that are micron-sized. This resulted in depressions uniformly spread over the silicone mould. It will be appreciated that in some other embodiments the depressions may not be strictly uniform. Acetone does not change the silicone surface structure, but it diffuses into the silicone causing it to swell up. This effect can be reversed by putting the silicone layer in an oven for 24 hours at 100° C. This thermal processing also improves the subsequent gold coating leading to cleaner gold on the surface of the silicone layer.

The resulting silicone layer was then coated with gold using direct current sputtering with a 99.99% pure gold target. The coating process was done with the rate of 0.5 nm per minute until a 12.5 nm thickness was achieved then the deposition rate was increased to 6 nm per minute (Q150R S, Quorum). This results in a gold layer that is at least 12.5 nm thick but may be up to 110 nm thick. In the preferred embodiments the thickness of the gold layer is between 30 and 110 nm thick. In the most preferred embodiments, the gold layer is 50 nm thick. This method first provides a uniform layer of gold on the silicone surface 3, then the higher deposition rate leads to a larger grain size of the gold layer 4 at the top of the structure with size distribution as shown in FIG. 6(g). This sputtering setting was used based on the simulation results detailed in relation to FIG. 4.

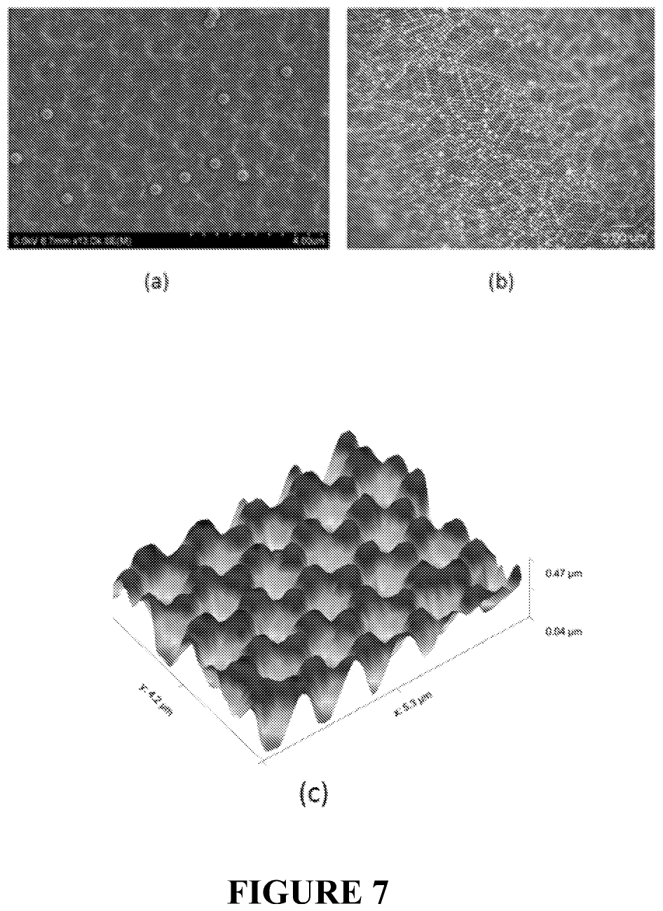

FIG. 7 shows the SERS substrate of Example 1. During creation, scanning electron microscopy (SEM) of 300 nm diameter polystyrene beads released on the SERS substrate was performed using a Hitachi SU-70 SEM to illustrate its trapping capability for EV-sized particles (FIG. 7 (a)) and optical microscopy demonstrates the consistent and tightly packed patterning of cup-shaped structures across the entire surface (FIG. 7 (b)). Atomic force microscopy (AFM) was also performed to further validate the nanocup structure of the fabricated SERS substrate using an Asylum Cypher ES AFM system, this is shown in FIG. 7(c).

### Example 1A—Alternative Fabrication of a Substrate (SERS Device) by a Combination of Soft and Nanoparticle Lithography

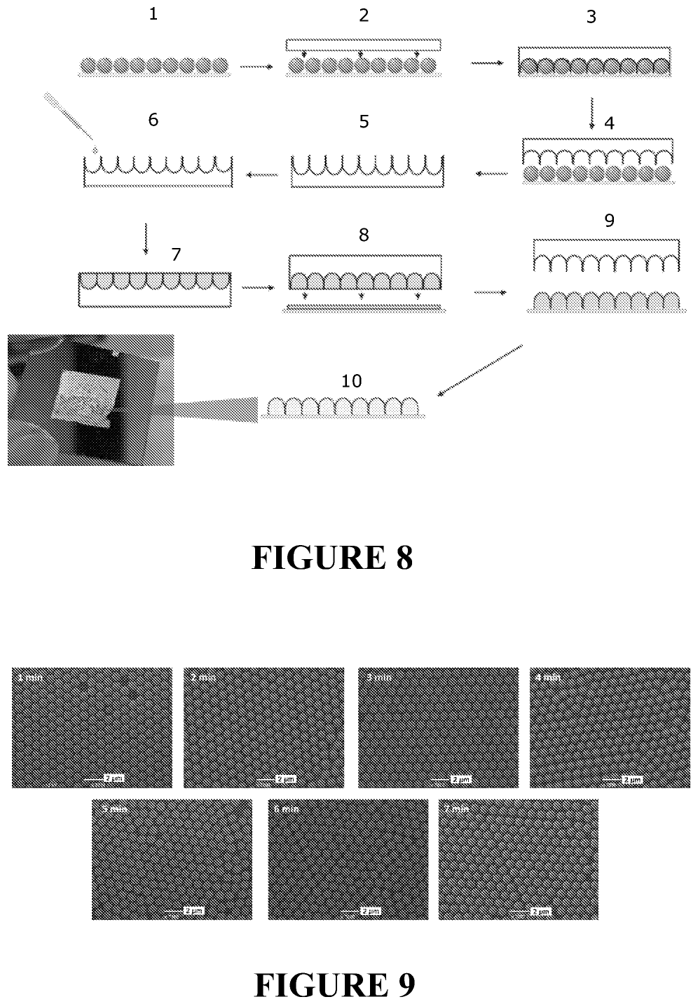

The method of example 1 can be adapted to provide a substrate or SERS device warped to provide a positive curvature rather than a negative curvature. Referring to the numbering in the schematic of FIG. 8, a suspension of 1 μm polystyrene nanobeads was deposited onto a glass slide (1), the suspension dried and a negative mould of the beads was created using PDMS (2) which was then baked (3) and the beads then removed (4), to create a silicone mould with a plurality of cup or bowl like depressions that are micron-sized (5), in a like manner to Example 1. This mould was then used as a PDMS stamp.

A glass slide with a thin layer of polystyrene anisole solution was placed on a 150° C. hot plate for 30 seconds to polymerize the polystyrene before being transferred to and kept on a 60° C. hot plate until use. During this process, the anisole was evaporated, leaving the polystyrene behind on the glass slide surface. A 10% polystyrene in anisole solution was spin-coated onto the PDMS stamp (6). The PDMS stamp was placed on the 150° C. hot plate for 50 seconds (7). After 50 seconds, the PDMS stamp was used to “stamp” the coated glass slide surface (8), by pressing the coated PDMS surface against the coated glass slide surface, applying a gentle and even pressure to the PDMS stamp for at least 10 seconds. The PDMS stamp was then peeled from the surface of the glass slide (9), and the nanodomes were coated with a gold layer (10) using a like process to that of Example 1.

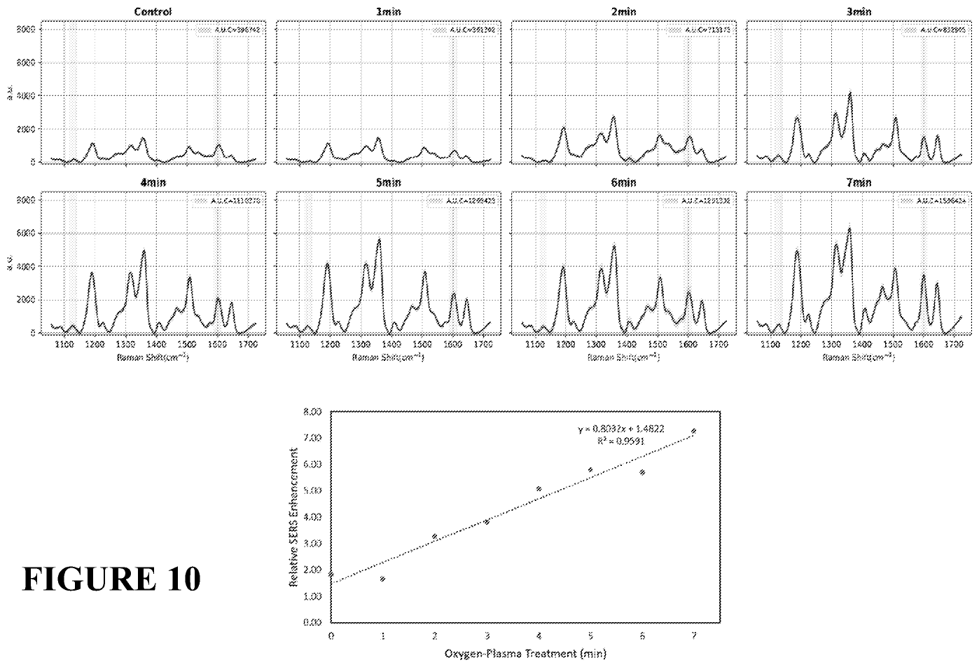

Once a number of nanodome substrates were formed, the substrates were oxygen plasma treated using radio frequency reactive ion etching (30 W, 0.7 torr, 10 SCCM O₂ flow rate) for different durations (1, 2, 3, 4, 5, 6, and 7 min). In FIG. 9, SEM images of the structure of the nanodomes is shown with progressively more oxygen plasma etching time, showing an increase in nanoscale curvature of the surface due to the formation of cracks randomly distributed along the surface. The increase in curvature resulted in greater SERS enhancement. This is illustrated in FIG. 10, which illustrates the SERS spectra of a 20 μL of 10⁻⁵ M R6G solution on each of the substrates shown in FIG. 9, and a control substrate which was not subjected to oxygen plasma treatment.

### Example 1B—Breast Cancer EV Isolation and Purification

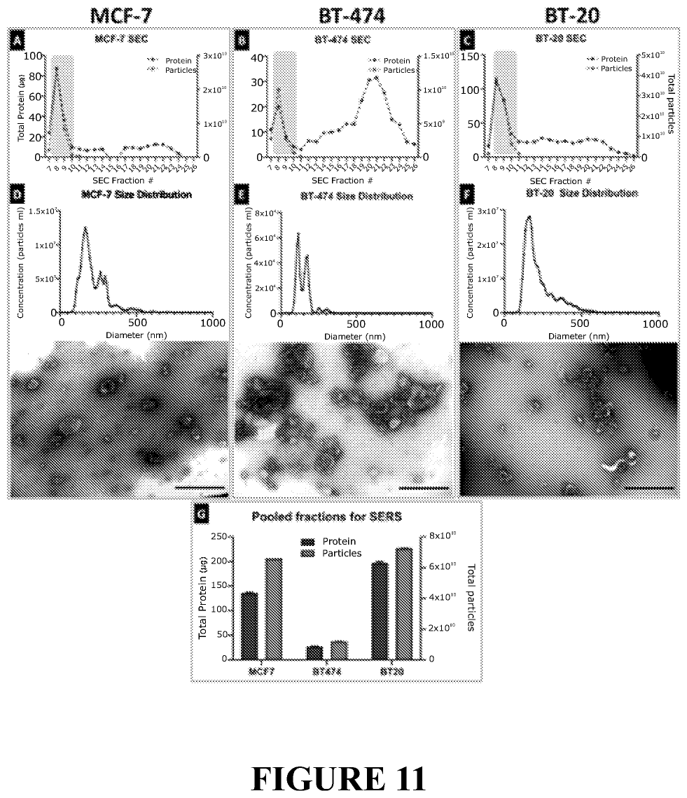

Based on NTA and BCA data, EV-rich fractions obtained using size-exclusion chromatography were found to be 8-10 (green/shaded boxes) and were pooled and used for NTA, TEM and SERS (FIG. 11A-C). NTA demonstrated the expected size distributions of purified small EVs from cell culture and TEM also showed a typical round morphology of the expected sizes (FIG. 11 (D-F)). Pooled EV samples (FIG. 11 G) were normalized to equal particle concentration using NTA measurements before loading onto SERS substrates. The use of CELLine AD 1000 bioreactor cultures combined with SEC purification provided several advantages compared to previous studies in this field. First, the 10 kDa semi-permeable cellulose acetate membrane separating the 15ml cell culture chamber from the 1L media chamber allowed the use of 2% FBS in the media chamber and passive diffusion of waste products and smaller nutrients between chambers, while preventing exogenous FBS EVs from entering the cell chamber. In addition, the chemically defined CDM-HD serum replacement additive further supported growth of the high-density culture. Often, EVs isolated from cultured cells are collected in one-off experiments, using large numbers of conventional flasks which require either inefficient FBS EV-depletion strategies or FBS starvation of the cultured cells, both of which could produce confounding factors in the final EV product. The bioreactors not only prevented these issues but provided a much higher concentration of EVs in the conditioned media compared to conventional cultures due to the high cell density. In addition, the natural turnover of cells within the bioreactor culture allowed the consistent harvest of EVs twice per week over the course of up to five months while replacing the media chamber only once per week. This is an advantage for the SERS fingerprinting and classification of the present invention, as the EVs are not seen as a limited or expensive reagent. The 15 ml of highly concentrated conditioned media also enables several efficient and cost-effective purification workflows, which are advantageous for rigour and reproducibility as the EV SERS field continues to grow. In this example, ultracentrifugation is used as a concentration strategy to reduce the 15 ml volume to 500 μl, followed by loading the sample onto a small SEC column to remove contaminating proteins (FIG. 11A-C). However, the initial 15 ml product could also be concentrated using ultrafiltration and used with a similar column, or even loaded directly onto a larger SEC column without concentration, depending on the equipment available.

### Example 1C—Reproducibility Testing of the Substrate

The functionality of the substrate of Example 1 was tested by obtaining Raman spectra from aqueous solutions of both Rhodamine b (Rb) and breast cancer EVs of different concentrations. Rb was used as a chemical standard to test the reproducibility of the designed SERS and to determine the degree of Raman enhancement caused by the surface of substrate Example 1 using a quick drying method, which is advantageous for EVs. Additionally, breast cancer EVs were investigated to demonstrate the potential of the fabricated SERS in cancer detection and classification. For the Raman measurements 1 μl of solution per square millimetre of samples was dropped on the fabricated SERS substrate of Example 1 which was quickly dried at 40° C. until no water was observed on the surface. This minimizes the coffee ring effect and increases the uniformity of the analyte across the SERS substrate.

### Example 1D—Quantitative Signal Enhancement

Mao et al. describe making a SERS substrate for single molecule detection²¹ with a feature size on the order of nanometres and with a fabrication process much more difficult than the SERS substrate of the present invention. The substrate of the present invention is better in terms of the electric field enhancement—The Mao enhancement factor is 3 compared to a flat surface while that of the substrate of Example 1 of the present invention is over 20. So, when considering that the Raman signal is proportional to the 4th power of the electric field this becomes a significant improvement over prior art SERS substrates.

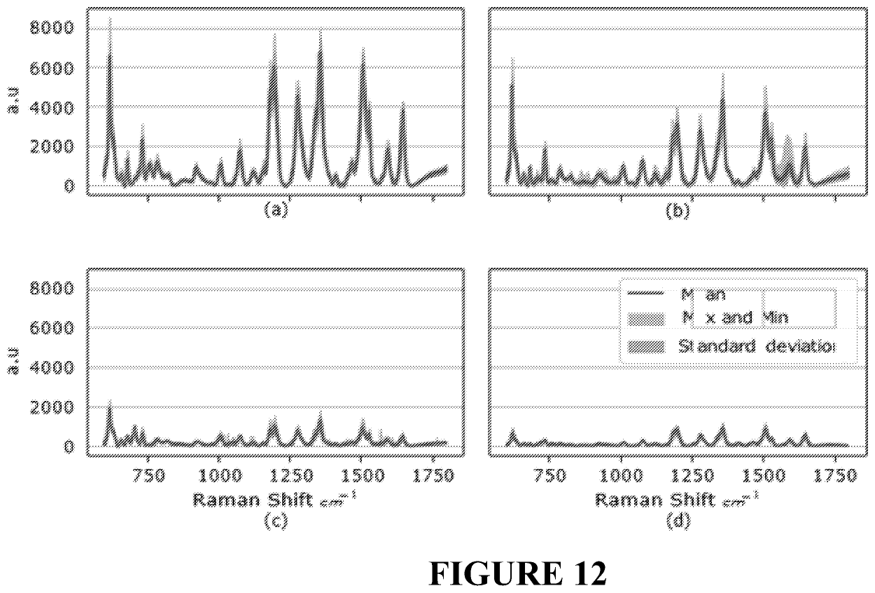

Sixteen spectra obtained from three different concentrations of Rb are shown in FIG. 12 (a), (b) and (c) for the concentrations of 10⁻⁴, 10⁻⁵ and 10⁻⁶ M, respectively. The yellow area corresponds to the maximum and minimum of the obtained spectra in each Raman shifts also the standard deviation is represented using cyan colour (inner shaded area). The curved substrate could identify the peaks of Rb even at 10⁻⁶ M while the lowest concentration that can be detected using a flat SERS substrate (FIG. 12 (d) was found to be 10⁻⁵ M, with almost half of the peaks' height. This corresponds to around 20 times amplification achieved by the curvature of the substrate, consistent with the prediction of FIG. 4 (b). Another important aspect of FIG. 12 is that it shows that the signal strength is directly related to the concentration, allowing quantification of the samples after proper calibration, which is valuable in biomedical applications.

### Example 1E—Reproducibility for EVs

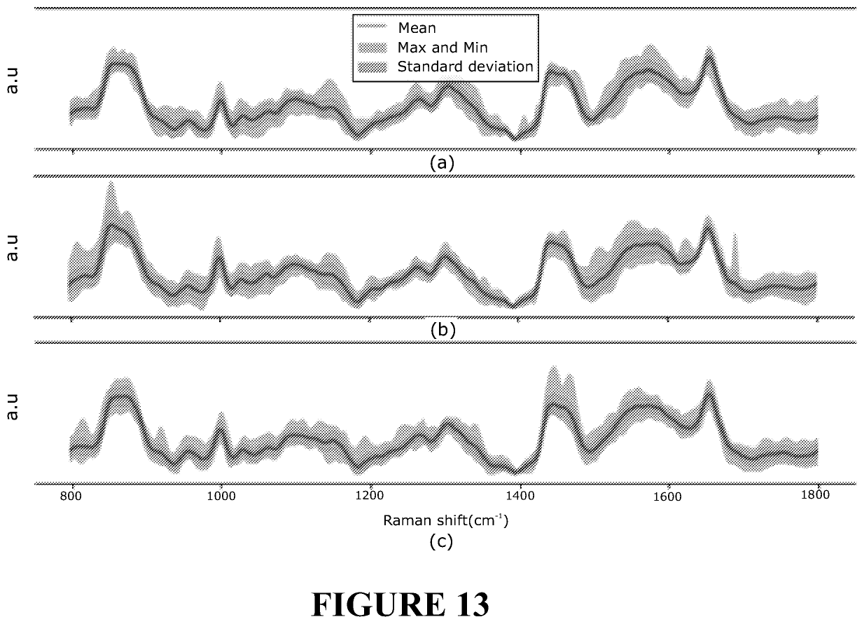

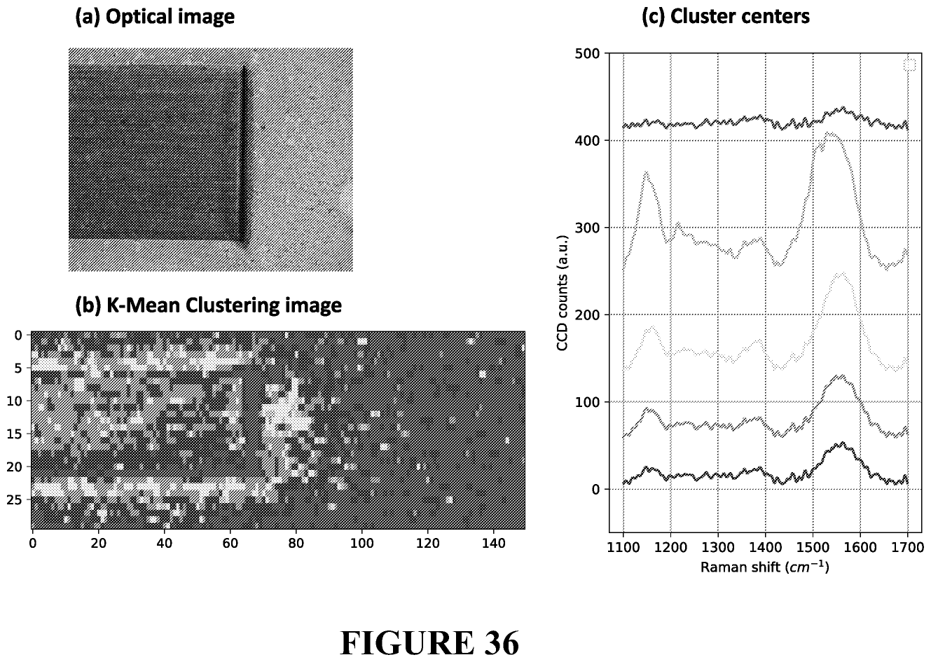

To test for reproducibility, Raman spectra of three replicates of EVs from the BT-20 cell line with a concentration of ≈1×10¹⁰ EVs/ml were obtained. Spectra from each sample were obtained from separately fabricated SERS substrates prepared according to Example 1, and 25 measurements were obtained from 25 different and random points where the minimum distance between sampling points was greater than the laser spot size. The normalized spectra from these three separately fabricated SERS substrates of small EVs from BT-20 cells are shown in FIGS. 13(a) to (c). Again, excellent reproducibility can be seen, indicating that any variability between substrates does not significantly affect the acquired spectra. The inventors also attempted to obtain SERS spectra from BT-20 EVs using a flat SERS substrate. However, due to the weak enhancement achieved by the flat substrate and the smaller hotspot areas (see FIGS. 2(c) and 5(b)), meaningful spectra could not be obtained (see also FIG. 36c, top trace which shows no SERS signal on the flat substrate).

### Example 1F—Testing Effect of Concentration on the Raman Spectra

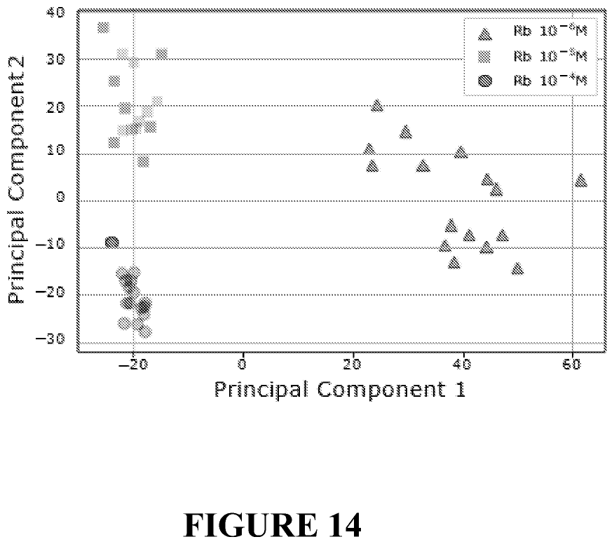

To demonstrate the effect of concentration on statistical analysis of the data we plotted the principal component analysis (PCA) of the normalized data from the Rb spectra of FIG. 12 in FIG. 14. The normalization removes the amplitude of the signal but preserves the wavenumbers of the different peaks. Although the major peaks of Rb almost perfectly match in the spectra, the PCA analysis shows some difference between samples. There are a number of reasons for this such as contamination during the fabrication process, and the difference in molecular orientation in hotspot areas. A small amount of contamination on the substrates can arise due to impurities of gold target, burning of the polymer (silicone/PDMS) due to the DC sputtering and the solution drying process. Variation in the drying speed can also lead to the presence of water in tiny geometrical features on SERS surface.¹⁹

### Example 1G—Effect of Concentration on the Raman Spectra—EVs

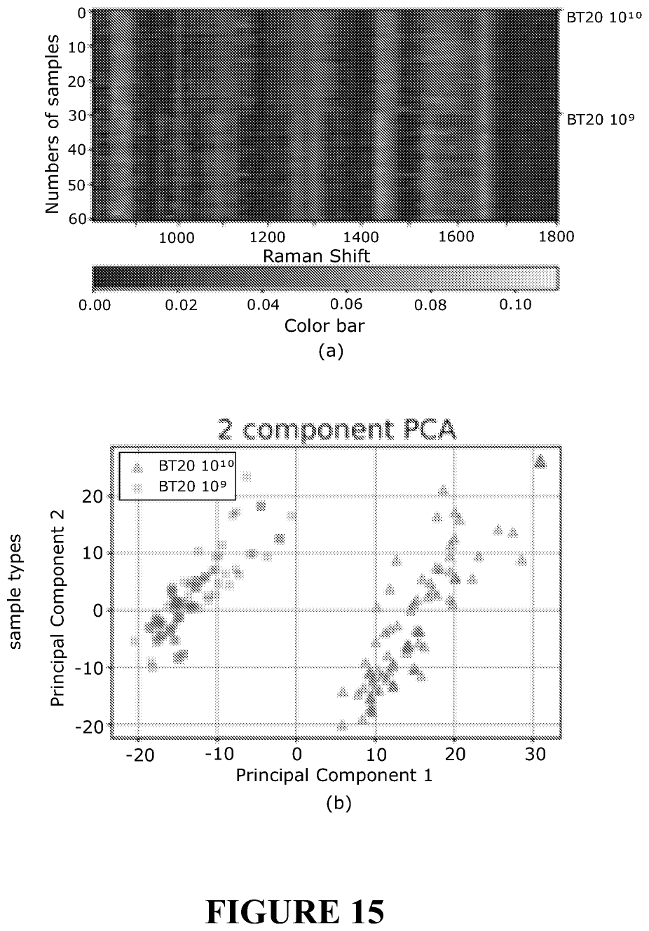

The effect of concentration was also investigated for the EVs spectra. The small EV solution from BT-20 cells was diluted to a concentration of ≈1×10⁹ EVs/mL and the spectra were obtained from 100 random points on the SERS substrate of Example 1. For the sake of clarity, 30 spectra of each of the 1×10⁹ and 1×10¹⁰ concentrations are shown in FIG. 15(a), and the PCA analysis is depicted in FIG. 15(b). Similar to the Rb spectra, the PCA shows there are some differences between the spectra, although the location of the major peaks remains the same.

### Example 2—Classification of EVs from Breast Cancer Cell Lines

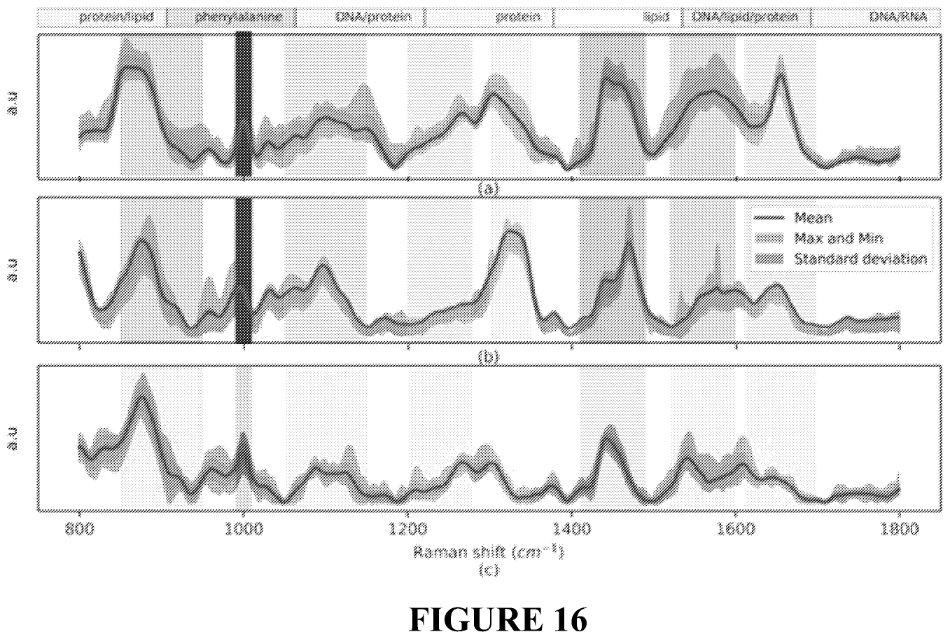

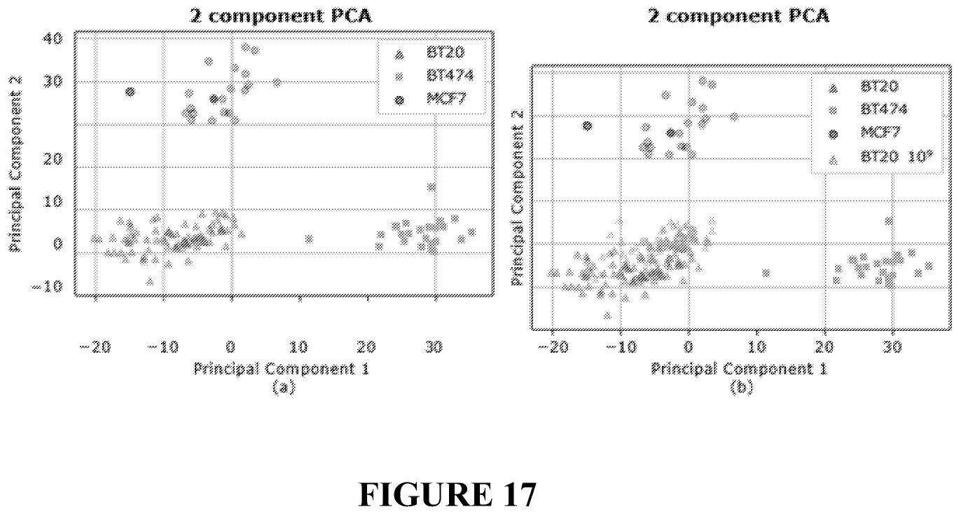

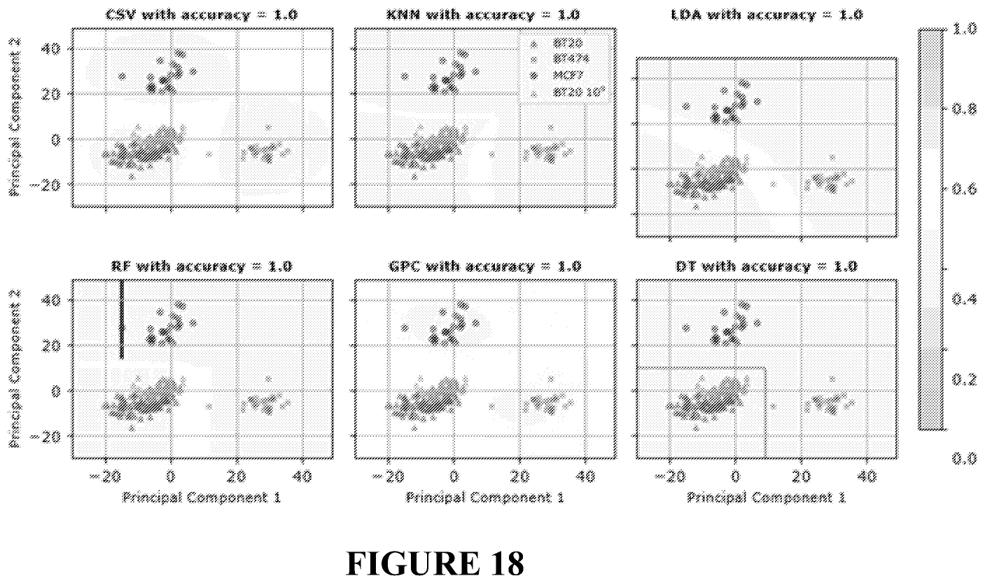

Using the SERS substrate of Example 1, the Raman spectra of EVs from two cell lines of other breast cancer subtypes, MCF-7 and BT-474, were obtained with equal concentration of ≈1×10¹⁰ EVs/mL to investigate the classification potential of the present invention. The average spectra of EVs of each type of breast cancer are shown in FIG. 16. In this figure the mean, maximum/minimum and standard deviation are shown in red (line), yellow (outer shaded area) and cyan (inner shaded area) colour, respectively and it can be seen that the sensing system again provides excellent reproducibility. In addition, each prominent band has been labelled according to the biochemical substance responsible for the peak following the analysis of Movasaghi et al.¹⁰⁰ The PCA analysis of these spectra is shown in FIG. 17(a). As can be seen from FIGS. 17 and 18, each breast cancer subtype can be easily distinguished leading to accurate classification using several algorithms.

As a demonstration of the potential of the method of the present invention, the same PCA transformation that was developed to compare the spectra of high concentration samples of BT-20 EVs with concentration of ≈1×10⁹ (i.e. ten times lower) was applied to the spectra of EVs from MCF-7 and BT-474 cell lines, and the results plotted in FIG. 17(b) along with the original data. As shown, the BT-20 EV spectra with different concentrations lie almost on top of each other and are well separated from the spectra of EVs from the other breast cancer subtypes. Importantly, this shows the method of the present invention can robustly classify breast cancer types irrespective of EV concentrations. Since patient plasma samples have reported EV concentration ranges anywhere from ≈×10⁷ to ≈×10¹³ EVs/mL and an average of ≈2×10¹⁰ EVs/mL,101 effective classification using only 20 μl of ≈ a sample of concentration 1×10⁹ EVs/ml clearly indicates that this approach is clinically viable. Often, 500 μl to 2 ml of plasma can be obtained per patient, and the number of EVs from that sample that would be analysed using SERS is highly dependent on any pre-purification steps, the specific patient, and whether the whole EV population is analysed or only a disease-specific subset of EVs.

To properly test the classification scheme, six different classification algorithms were applied, all trained on the high concentration samples within the data set. The algorithms used were: C support vector classifier with RBF kernel (CSV), k-nearest neighbours (KNN), linear discriminant analysis (LDA), Random forest (RF), Gaussian process classifier (GPC), and decision tree (DT). The results are shown in FIG. 18 with the probability of each point in PCA plane to be BT-20 being represented using a colour map. All the chosen classification algorithms successfully identify all the EV spectra correctly including BT-20 at a lower concentration. This shows the power of this sensing system of the present invention to classify the EVs at lower concentration, which is 100 times more sensitive than ELISA, a standardized fluorescence or absorbance-based microplate assay usually performed using antibody-conjugates.⁶⁴

While the methods described herein enable the preparation of highly homogenous and purified suspensions of breast cancer EVs, in patient samples, the number of breast cancer EVs relative to other cell type-derived EVs could be very limited. However, these results show that the Raman spectra of EVs may serve as valuable diagnostic or monitoring tools in breast cancer and many other diseases.

### Example 3—Identification of EVs from Bacteria

Machine learning was applied to SERS spectra obtained using the label-free SERS substrate of Example 1, to classify E. coli EVs based on differences in strain, culture conditions, and purification method. This example establishes manifold machine learning as a viable method of dimension reduction for EV SERS spectra. Importantly, the ability to classify E. coli EVs based on these parameters is unlikely to preclude their collective or individual classification from EVs produced by other cells, such as host EVs in the case of infection. Rather, this example demonstrates the ultra-sensitivity of SERS for bacterial EV fingerprinting, as even slight differences in acquired spectra can be used for classification following the application of various machine learning algorithms. Thus, SERS can be used for bacterial EV characterisation in a multitude of laboratory and clinical applications.

### Example 3a—Bacterial EV Characterisation

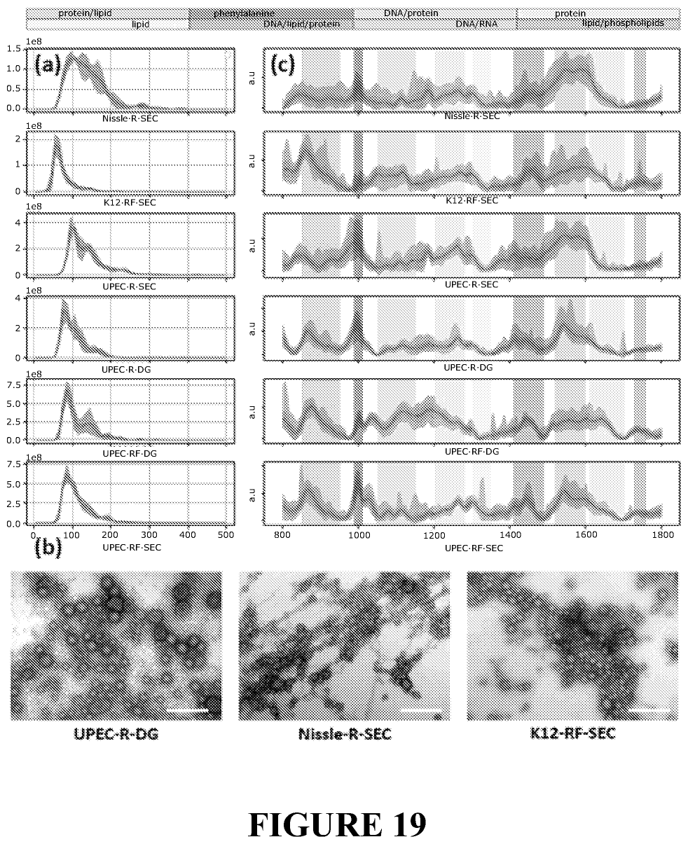

Nanoparticle tracking analysis (NTA) data demonstrated that all UPEC EVs exhibited similar sizes, with an average mean of 115.4+/−6.5 nm and mode of 89.7+/−4.5 nm (FIG. 19 (a)). Interestingly, the probiotic Nissle-R-SEC EVs were noticeably larger with a mean diameter of 135.8 nm and mode of 101.2 nm, while K12-RF-SEC yielded smaller EVs mean diameter of 71.1 nm and a mode of 62.9 nm. TEM imaging demonstrated similar EV morphologies and the expected size range for all samples, and representative TEM images for EVs from each E. coli strain are shown in FIG. 19 (b).

The post-processed SERS spectra for all E. coli EVs investigated are shown in FIG. 19(c). These spectra were normalised and thus any information related to the amplitude of SERS spectra were removed. The mean, maximum/minimum and standard deviation around the mean was calculated and presented with red (line), yellow (outer shaded area) and cyan (inner shaded area) colour, respectively. The correspondence of Raman bands to the known types of biomolecules are shown using vertical colour bands.¹⁰⁰

### Examples 3B-3G—Classification Using Multivariate Analysis and Machine Learning

Samples were classified using standard machine learning algorithms in two different scenarios. In the first scenario, data from the three relevant pairwise comparisons based on strain, culture medium, or purification method were used for training and classification. This analysis was performed first to determine the effect of each individual parameter at a time. In the second scenario, the machine learning algorithms were trained to identify the EVs from all 6 subtypes simultaneously, such that the spectra of all EVs were used for training.

### Example 3B—Paired Subset Classification

For the purpose of strain classification, paired subsets {Nissle-R-SEC, UPEC-R-SEC} and {K12-RF-SEC, UPEC-RF-SEC} were used as they are different strains but were grown in identical culture medium and purified identically. Similarly, the paired subsets of {UPEC-R-DG, UPEC-RF-DG} and {UPEC-R-SEC, UPEC-RF-SEC} were used for the investigation of culture medium as they are the same strain and purified identically but grown in different culture media. Lastly, {UPEC-R-SEC, UPEC-R-DG} and {UPEC-RF-SEC, UPEC-RF-DG} were used for the evaluation of purification methods as they are the same strain and grown in identical culture medium but purified using different methods.

To effectively present the variance of the obtained SERS spectra all the components of each spectra was investigated. Each of the obtained spectra consists of 1512 data points between Raman shifts of 800-1800 cm⁻¹. These are, in fact, an array of 1512 dimensions and the vast number of dimensions severely limits the visualisation of the variance within the data. Principal Component Analysis (PCA) was used to transform the obtained spectra in a way which reduces the spectral dimensions while preserving the maximum variance between the data after transformation. This was done firstly for the sake of the visualisation and secondly as means of classification in lower dimensions between samples.

### Example 3C—Automatic Classification by Machine Learning Algorithms

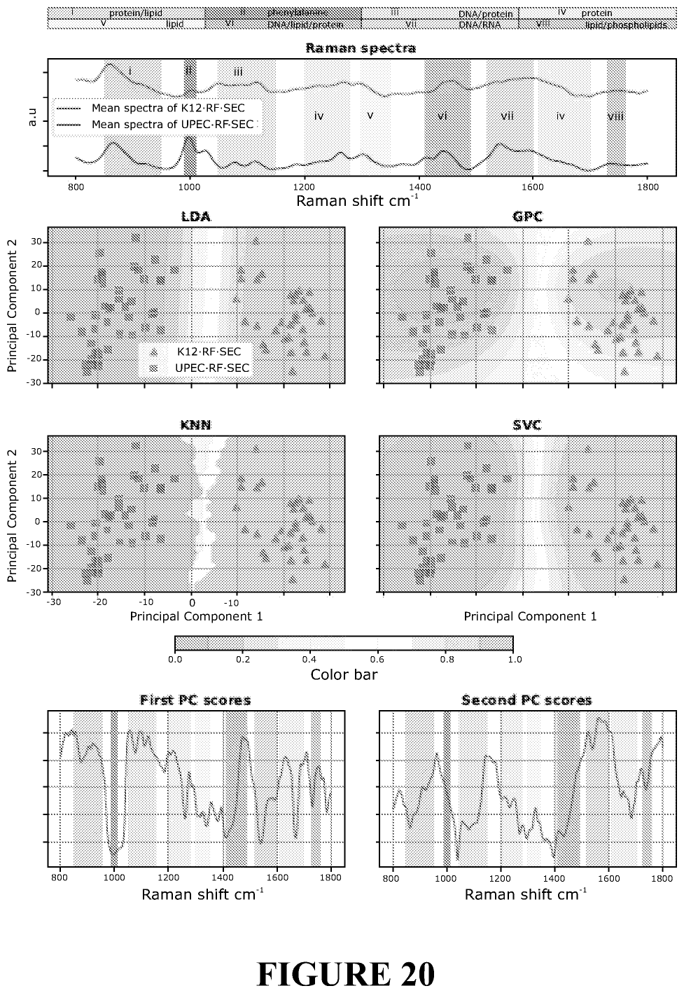

To demonstrate the possibility of the automatic classification, we used four different types of established machine learning algorithms, including Linear Discriminant Analysis (LDA), Gaussian Process Classifier (GPC), K-Nearest Neighbour (KNN), and Support Vector Machine (SVM) with RBF kernel. All the machine learning algorithms were employed over the PCA transformed data to calculate the probability of each point in the PCA plane to be classified as the correct type within each subgroup. The average SERS spectra for K12-RF-SEC and UPEC-RF-SEC, PCA transformation, and probability distribution obtained using machine learning algorithms, as well as first and second PCA scores are depicted in FIG. 20.

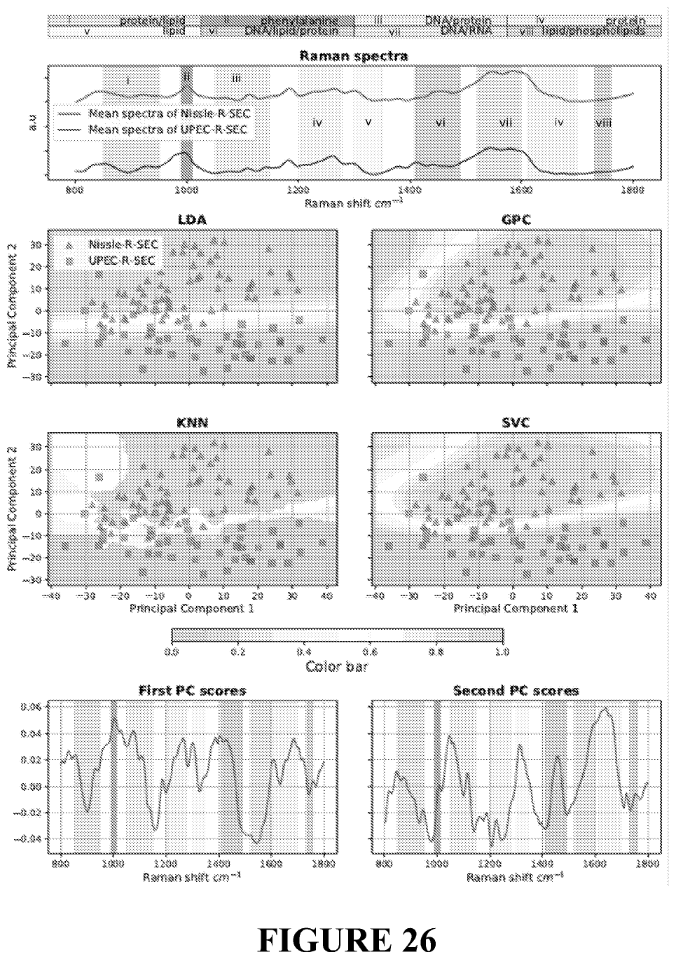

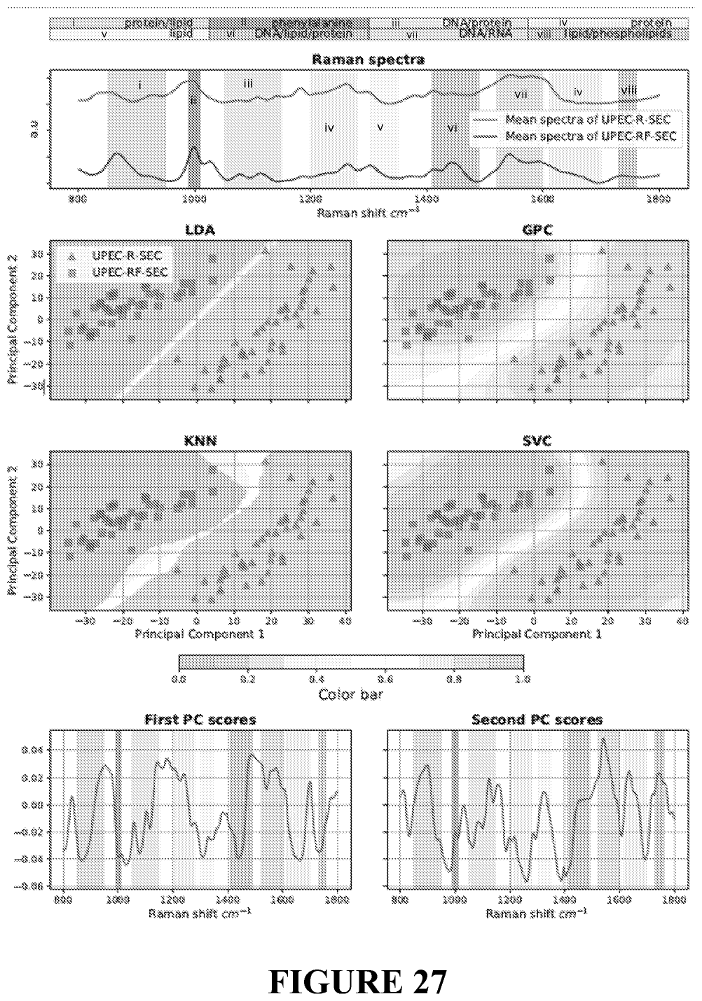

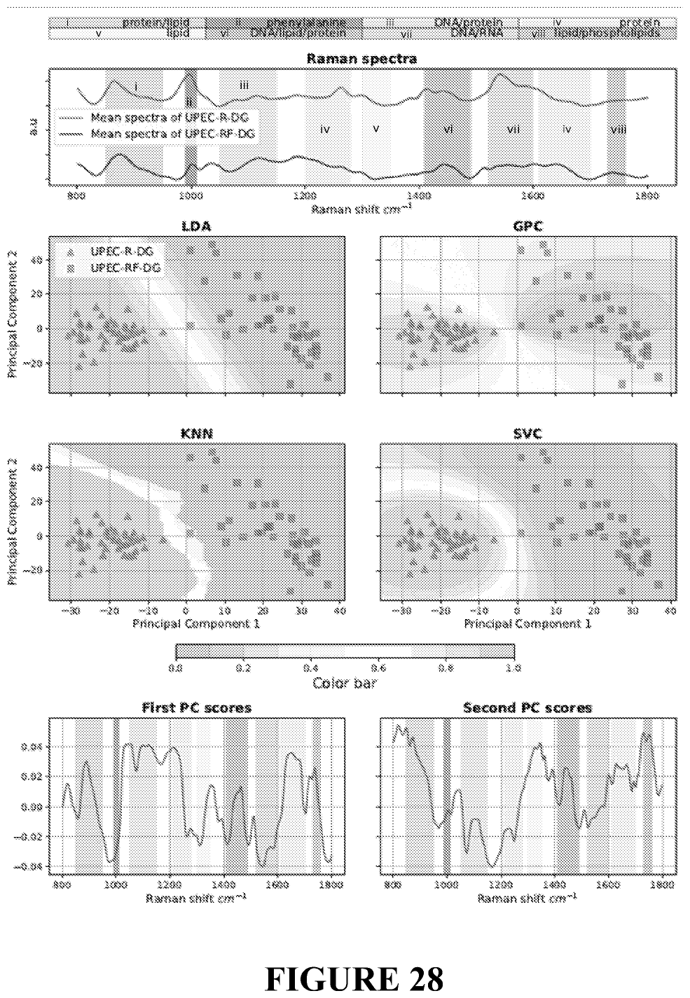

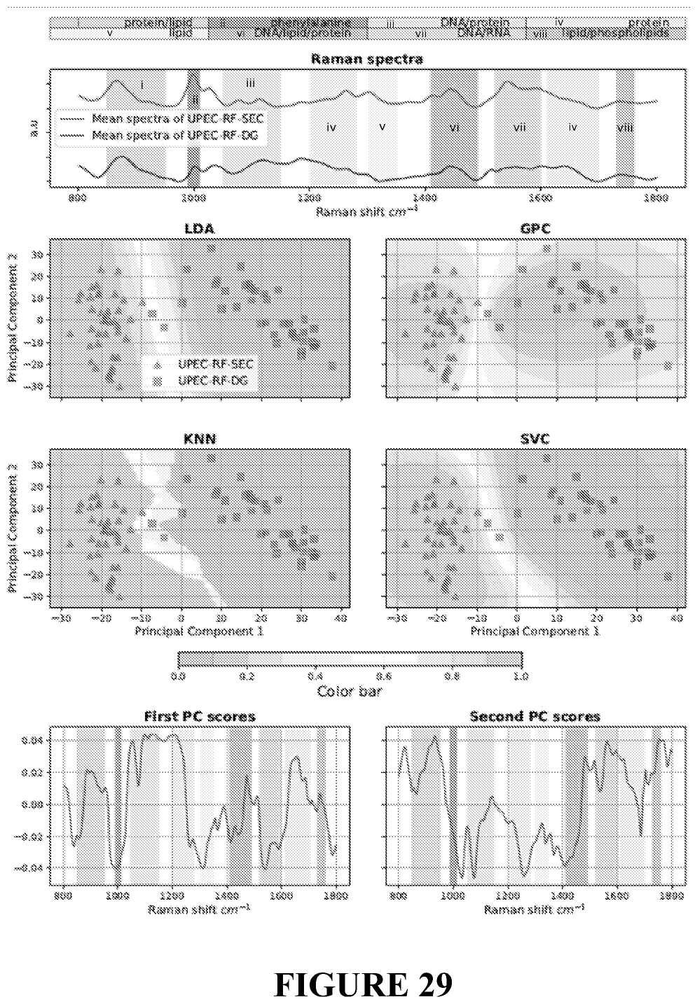

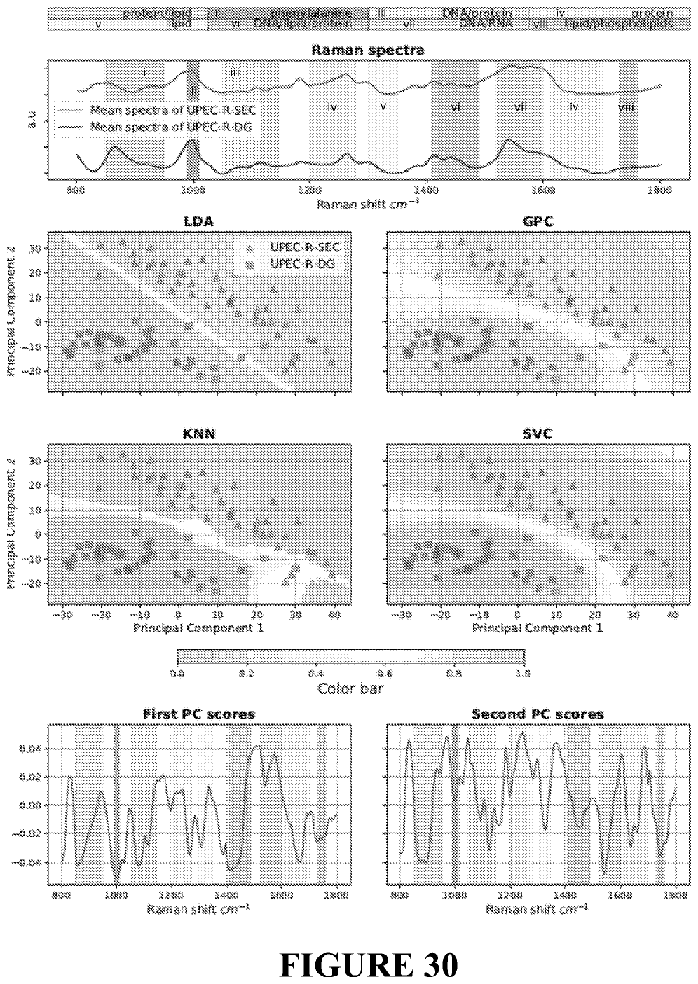

For the paired comparison of K12-RF-SEC and UPEC-RF-SEC in FIG. 20, clear differences in several peaks can be visually discerned, resulting in an expected and highly efficient classification by all the machine learning algorithms tested. By comparison, relatively subtle differences can be seen in the averaged SERS spectra for Nissle-R-SEC and UPEC-R-SEC (see FIG. 26), and while the classification is not as efficient, all the machine learning algorithms tested were still largely effective. For pairwise comparisons between plain and iron supplemented culture media (see FIGS. 27 and 28), clear differences in several peaks can be easily discerned, resulting in highly efficient classification in both cases. Lastly, differences between SEC and DG-purified UPEC EVs from both types of culture media can also be effectively classified (see FIGS. 29 and 30), although not quite to the level of efficiency as when comparing different culture media.

### Example 3D—Classification of All Samples

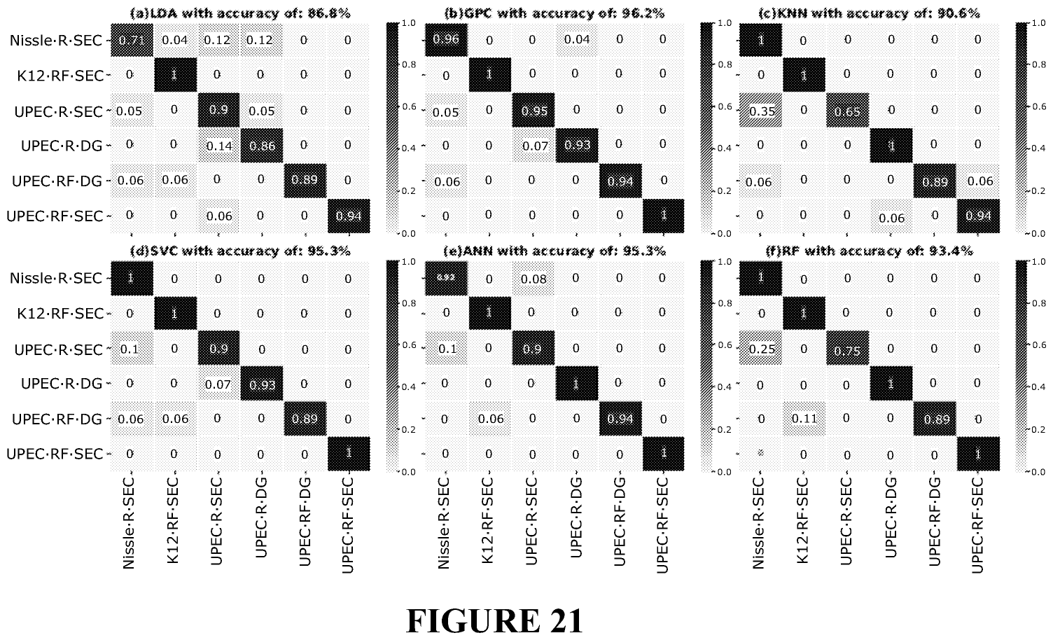

Based on the initial success of classification using simple pairwise comparisons, a more challenging investigation of classifying all the samples simultaneously was explored. For the purpose of whole data classification, we first trained all the machine learning algorithms over each of the normalised spectra used to produce FIG. 19(c) without PCA transformation. The confusion matrices produced when 60% of the data was used for training and the other 40% was used as a testing set are shown in FIG. 21 for all the chosen machine learning algorithms used previously, in addition Artificial Neural Network (ANN) and Random Forest (RF). Of the six classification methods tested, five produced efficiencies above 90%, with GPC, SVC, and ANN all resulting in greater than 95%. LDA performed the worst, but still produced a classification efficiency of 86.8%. Collectively, these results demonstrate the power of analysis of SERS spectra with machine learning, as even subtle differences between normalised bacterial EV fingerprints can enable highly efficient classification, even from the same species of bacteria.

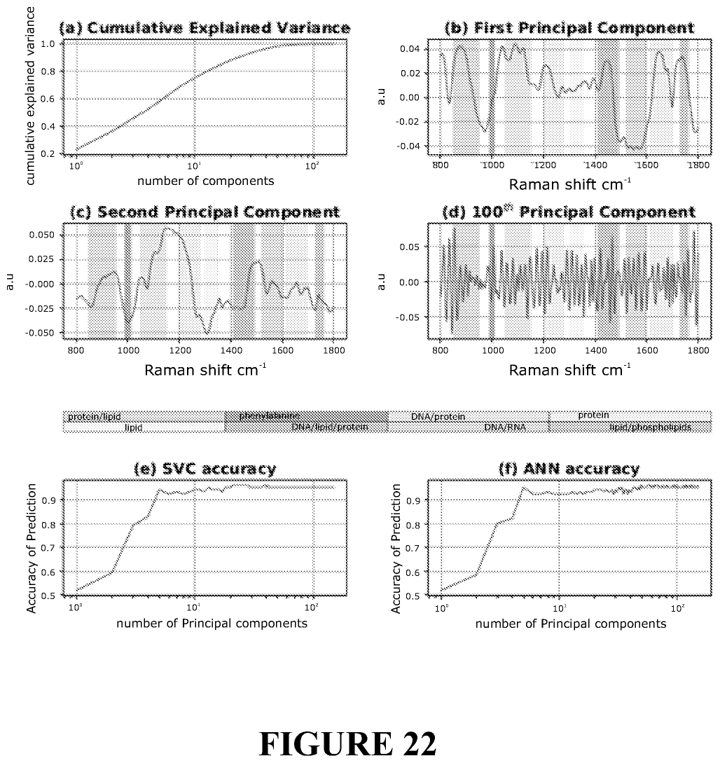

The same algorithms were also performed on the PCA-transformed data. Cumulative explained variance and the first, second, and 100th PCA scores are depicted in FIG. 19 (a), (b), (c) and (d), respectively. The accuracy of SVC and ANN machine learning algorithms when different PCA dimensions were considered for training are shown in FIG. 22(e) and (f), demonstrating that the acceptable accuracy is achieved when few (5-10) PCs are used. Interestingly, increasing the number of components beyond a certain point does not lead to better classification result. One of the main reasons for this phenomenon may be the natural variance in the E. coli EV biochemical contents within each subtype and the noise level due to weak Raman intensity of EVs. In fact, the 100th PC depicted in FIG. 22(d) contains little biochemical information about the EVs and thus adding more PCs for classification purposes can actually lead to weaker classification results.

### Example 3E—Effects of Culture Medium on SERS Spectra

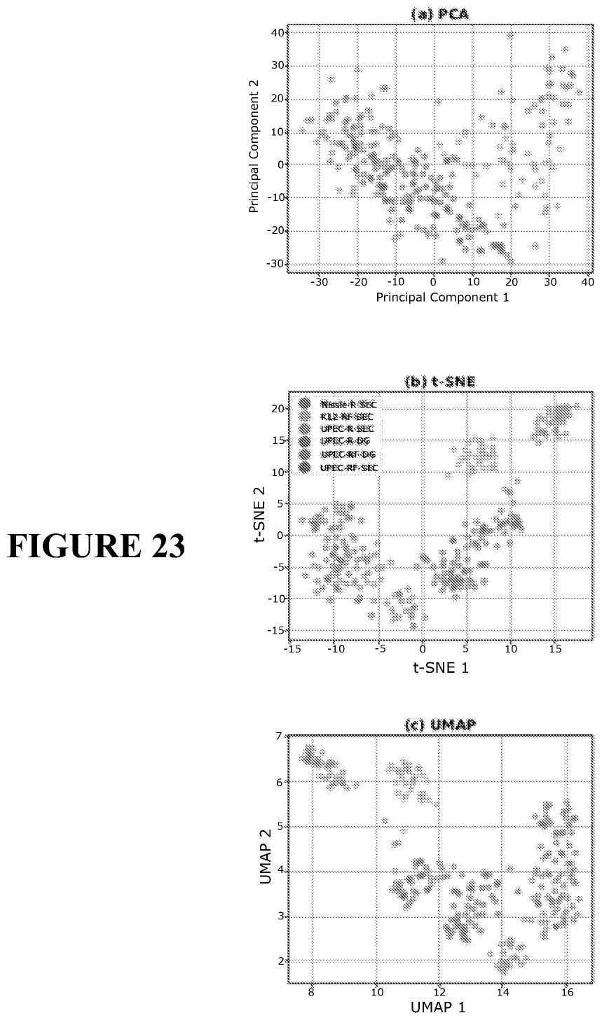

To further investigate the effect of culture medium on the SERS spectra, which is known to significantly affect the proteome of E. coli EVs according to a previous study,⁸⁶ two dimensional PCA transformation of the whole data set is plotted in FIG. 23 (a) with the plain (R) and iron(III) chloride-supplemented (RF) medium marked by {red, green, blue} and {purple, orange, brown}, respectively. Clearly, the first principal component can effectively separate the data of E. coli EVs produced in the different media. This is likely due to the fact that the SERS spectra of EVs is mostly determined by the surface biochemical makeup of the EVs, which appears to be heavily influenced by the culture medium. For this study, the spectra below a Raman shift of 800 cm⁻¹, which would contain peaks related to iron, were removed to focus on the EV biochemical contents rather than any free iron bound to the EV surfaces. This strong influence of culture media on the SERS spectra not only corroborates the previous study,⁸⁶ but indicates that the type of fluid in which bacterial cells are cultured may need to be carefully considered in future EV SERS comparisons. This could be particularly important in clinical applications, as different bodily fluids may confound specific species or strain identification.

### Example 3F—Manifold Machine Learning for Improved Data Visualisation

As shown in example 3E and FIG. 23 (a), 2-dimensional PCA transformation cannot effectively illustrate the variance of the data as the data points of each subgroup are mixed with no clear spacing between them. From the results of the classification algorithms using PCA-transformed data in FIG. 22 (e) and (f), we can see that the 5-dimensional PCA transformation, however, results in a very good classification accuracy. In other words, in 5 dimensional PCA space, the data corresponding to each sub-type of E. coli EVs are effectively separated with clear boundaries between them. Given the challenges conceptualizing anything beyond 3 dimensions, a better dimensional reduction technique could ideally reduce the dimensional complexity. To achieve this, the inventors employed t-SNE¹⁰² and UMAP¹⁰³ as unsupervised manifold machine learning and non-linear dimensional reduction techniques, with the results presented in FIG. 23(b) and (c), respectively. In clear contrast to the standard PCA transformation (FIG. 23(a)), the variance and differences between the spectra of each E. coli EV subtype are much clearer in two dimensional UMAP and t-SNE planes.

### Example 3G—Alternative Method of Analysing Spectra—Convolutional Neural Networks

In the system of the present invention a CNN may be used to classify or analyse the spectra obtained. The CNN of the present invention is inspired from ResNet architecture and then the network trained network using two different data sets. The primary data set used was a set of Raman spectra of different human bladder cancer tissues. This data set contains 2592 spectra obtained using portable, low resolution Raman spectrometer for three categories namely as healthy, low-grade and high-grade tumour. This data was obtained using various laser's power therefore it contains Raman signals with the wide range of signal to noise ratios (SNR) and baselines. The second data set used the SERS spectra of EV from different strains of EV from E-Coli. SERS peaks of EVs are known to be very weak due to absence of chromophore molecules in them. The other main issue regarding them is natural SERS's Raman bands which can be easily interfere with the bands of EVs and therefore usually require explicit human interaction for pre-processing.

The two data sets were aggregated to avoid overfitting of training and to introduce different scenarios to the proposed network to obtain more reliable accuracy when the network is faced with data obtained from different Raman spectrometers.

The CNN of the present invention is a feed forward neural network with added skips which bypass some layers in between. This is utilized to overcome two main obstacles toward having a very deep neural network. Firstly, it significantly helps the problem of vanishing gradient and makes it possible to implement a much deeper neural network. Secondly, accuracy saturation and degradation, which is another problem of deep plain neural nets, has been successfully handled with this technique. Accuracy saturation arises when more layers are added to a shallow plain neural network. Initially, adding more layers to the network increases the accuracy but then it gets saturated and finally degraded rapidly after a point.

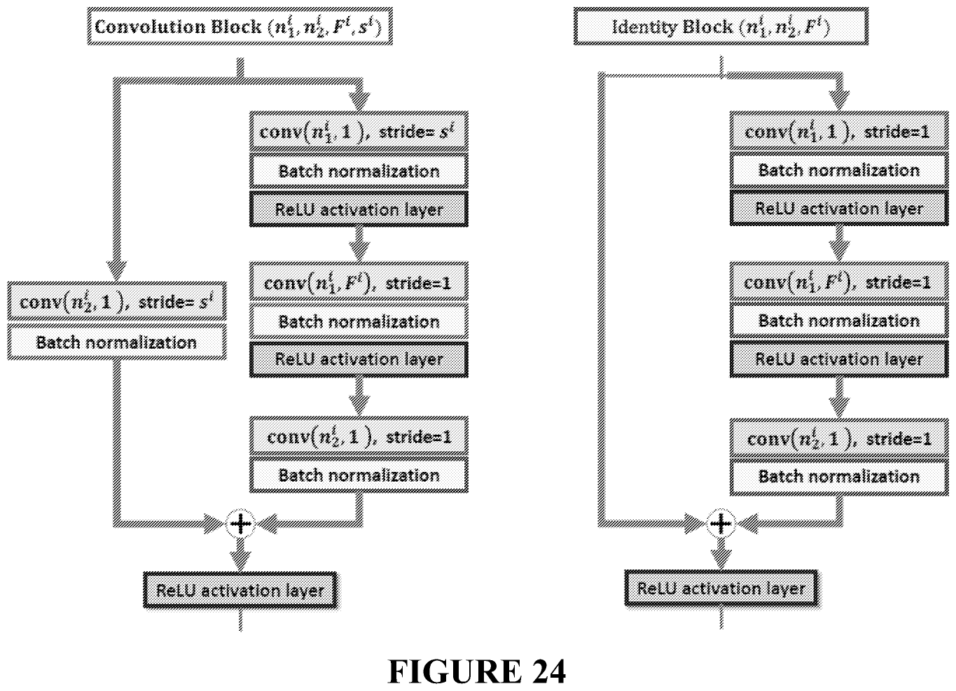

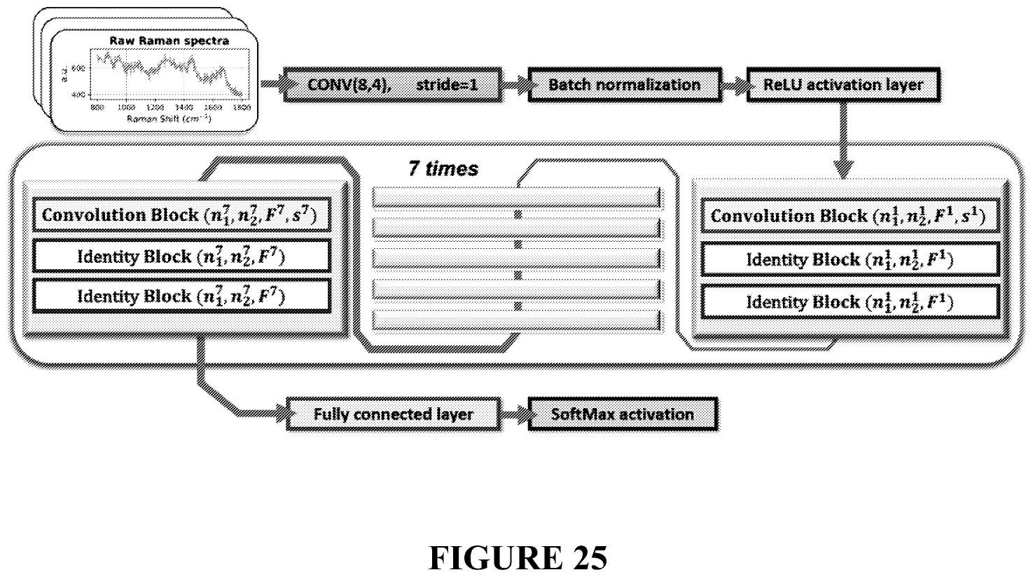

The skips are implemented in two sub blocks of the whole network namely as Identity block (see FIG. 24) and Convolutional block (see FIG. 25). Each of these blocks consist of some stacks of convolution, batch normalization and nonlinear activation layers while the input of them is shortcut to their output. The only difference between them are the presence of a single convolution and batch normalization layer in the detour pass of the convolutional block. The parameters nⁱ₁ and nⁱ₂ are the number of filters in the different layers of convolution layers (CONV) while Fⁱ and sⁱ are the size and stride step defined for the filters.

The overall architecture of the CNN of the present invention is depicted in FIG. 25. The training data set is the raw Raman spectra. This data is directly feed into a convolution layer followed by batch normalization and the rectified linear activation layer. The output of the activation then is feed into a seven consecutive step process. Each step consists of a stack of Convolution blocks followed by two Identity blocks. Finally, the output of the last step is connected to a fully connected flat layer and the output of the whole network is generated using a softmax activation layer.

The classification ability of the CNN of the present invention was compared to a number of known machine learning algorithms and each of the algorithms robustness to change in the spectroscopy setup and weak calibration. The CNN of the present invention resulted in an accuracy of 94.3%, which compares well with the best result previously obtained by machine learning technique (96% using Gaussian process classifier). Thus, it can be seen that the CNN of the present invention overcomes disadvantages of other machine learning techniques, in that human intervention is not required as well as reducing the effects of weak EV spectra and diversity of spectra. As such, extensive baseline corrections and denoising is not needed, reducing time, effort and vulnerability to human error.

### Example 4—Substrate (SERS Device) by Laser-Induced Nanostructuring of SERS-Active Thin Films (LINST)

### Example 4A—Formation of LINST

With reference to the protocol mentioned in the Materials and Methods section above, the laser scanning strategy was tailored to very low fluence (slightly above the ablation threshold) and the distance between scanned lines was optimized to continuously pattern the whole area. In this example, LINST was carried out with a fluence of 0.2 J/cm², scanning speed of 1.125 mm/s, and a separation between the scanned lines of 2.5 μm.

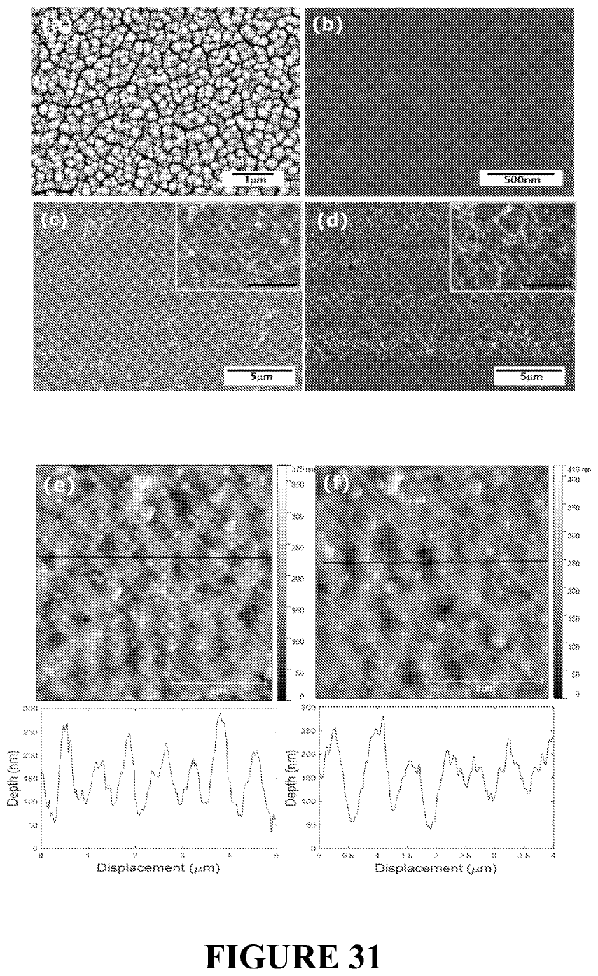

Both magnetron sputter coated, and thermal evaporated gold thin films were tested for their potential use in LINST patterning. As shown in FIG. 31a-b, the initial grain size was different for each method as well as the overall thin film morphology at the nanoscale. However, following fs-laser-machining, the LINST were almost identical in terms of nanotopography (FIG. 31c-d). Following irradiation, both types of gold thin films were fully covered by nanostructures with no areas remaining where the gold thin films were not patterned. As expected, the initial differences in grain size did not play a major role in the fs-laser nanopatterning process since the absorption of both surfaces was very similar.

The presence of slightly periodic nanoripples oriented perpendicular to the scanning direction can also be seen, which somewhat resembles the LIPSS method that is used for patterning bulk materials. 2D-FFT analysis of the SEM images indicates that the fabricated patterns present an average periodicity of 565±20 nm and 548 nm±27 nm (FIG. 31e-f) which coincide with a periodicity slightly smaller than the irradiation wavelength. Additionally, AFM images and profiles (FIG. 31) corroborate these findings and indicate the presence of both convex and concave curvature, around 150 nm deep, that exist across the entirety of the patterned surface. Given the known thickness of the deposited gold layer, the AFM results verify that the gold thin films (substrates) were not fully ablated at any point, avoiding any risk of the underlying chromium or silicon base layer contaminating the SERS signal.

Both SEM and AFM images clearly show the presence of small gold nanoparticles (tens of nanometres) covering the entire patterned area, a phenomenon associated to the redeposition of the thin film material ejected during modulated ablation (FIG. 31c-d insets). Once it was established that either deposition method could be easily used for LINST patterning, thermal evaporation was used for the remainder of the study. The inventors believe that other deposition methods known in the art may also be used. And other suitable deposition methods will be apparent to those skilled in the art.

Most recent studies about LIPSS conclude that the formation of such structures arise from a combination of plasmonic interference effects and hydrodynamic matter reorganization.⁷ In the case of LINST, the need to nanopattern the surface while completely avoiding bulk removal of the thin film led to the use of a very low fluence and relatively low number of pulses. These conditions made LINST patterns less defined and coherent compared to conventional LIPSS, but the resulting periodicity combined with the gold nanoparticle redeposition ultimately produced viable SERS-active substrates. While the redeposited nanoparticles can often be detrimental in laser machining applications, they are actually ideal for SERS purposes as demonstrated in the following sections.

Moreover, LINST may be done in a single fabrication step, avoiding the need of additional materials or chemicals or the use of cleanroom environments since the process is performed in an open-air atmosphere. Lastly, the potential scalability with the use of the latest femtosecond lasers and machining platforms could allow the fabrication times to be reduced by even 100-fold compared to the minimal time required for the examples herein.

### Example 4B—Full-Wave Simulation

As seen in FIG. 31, the gold LINST substrates contain both concave and convex curvatures on their surface, with gold nanoparticles scattered substantially across their entirety. To quantify the potential effect of these combined features on the Raman signal enhancement, simulations were performed in COMSOL Multiphysics based on the AFM profiles. It is important to note that both substrate curvature and nanoparticle sizes have a seemingly random nature within specific size ranges. To effectively assess this complexity, the simulations were performed over the actual dimension ranges of nanoparticle sizes and substrate curvatures.

Given the extensive possible combinations of particle sizes and substrate curvatures, the three-dimensional structures were simplified to two dimensions. This was done to reduce the computational cost while still exploring the fundamental characteristics of the LINST structures. A small portion (10 nm) of the nanoparticle was also fused into the substrate to avoid a singularity at their intersection and to simulate a more realistic physical condition (FIG. 32a).

As previously established, plasmonic nanostructures can confine incoming light around their nanometric features and sharp edges (also known as hotspot). The confined light is then presented with higher electric field amplitude, effectively enhancing any nonlinear phenomena such as Raman scattering. To quantify this effect for various geometries and conditions, the area of regions which have an electric field enhancement of more than 5 fold (corresponding to more than 5⁴ fold Raman enhancement) was calculated as shown FIGS. 32b and 32c for 532 nm and 785 nm excitation wavelengths, respectively. According to the simulations, the 785 nm laser leads to 100 times larger hotspot for most of the particle sizes and substrate curvatures that were simulated. This should in turn lead to a better Raman signal enhancement for larger molecules and EVs on the LINST surface, as they can be better fit within the hotspot areas. The normalized electric field to the incident field for the instances which achieved the best hotspot area for 532 nm and 785 nm are also presented in FIGS. 32d and 32e, respectively.

### Example 4C—Measuring SERS Performance

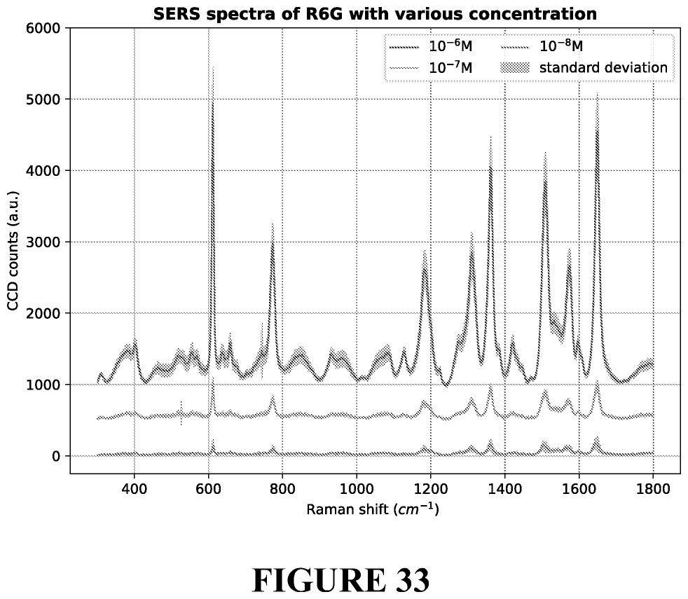

To clearly demonstrate the performance of the optimized LINST for SERS, we obtained Raman spectra of various Rhodamine 6G (R6G) concentrations using 532 nm laser and 1 second acquisition time with a 50×microscope objective. The average of 100 spectra at each concentration, and their standard deviation, are shown in FIG. 33.

This demonstrates the high sensitivity of the fabricated SERS as all the R6G bands are clearly observable even at a concentration of (10⁻⁸ M). There is also a clear quantitative relationship between R6G concentration and the amplitude of the signal, meaning that with a proper calibration, it could potentially be used to gain concentration information of the sample from the acquired SERS spectra alone.

### Example 4D—SERS Spectral Quality Improvement

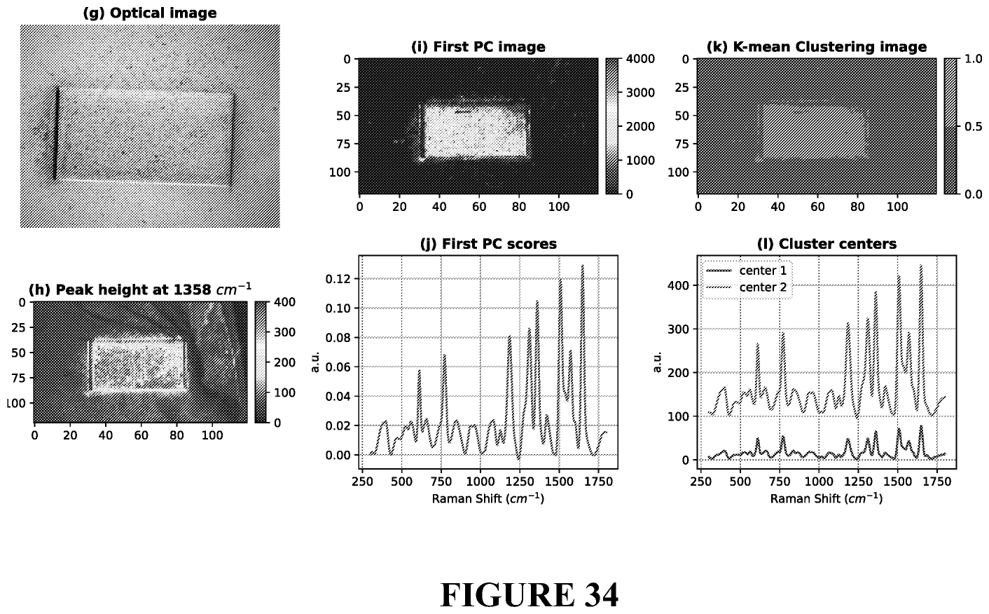

Hyperspectral Raman imaging was also used to compare the sensitivity of the LINST area to the area which was not directly machined. For this image, the 532 nm laser with 50×microscope objective and R6G with concentration of 10⁻⁷ M were used, but as a relatively large grid (100×100 μm) was imaged, the acquisition time was reduced to 100 ms. As shown in the optical image of the investigated sample (FIG. 34a), an area which contains the boundary between the machined area and bare gold was chosen to determine if any correlation between machining and obtained spectra quality existed. In FIG. 34b, the amplitude of the obtained spectra at 1358 cm⁻¹, one of the main R6G peaks, is shown. Although this slightly higher amplitude of the peak of R6G over the machined area shows a better performance of the machined area, the information regarding the other peaks and hence the overall quality of the obtained spectra is not clearly demonstrated.

To have a better understanding of the overall quality of the acquired spectra rather than a single peak's amplitude, the first principal component (PC) was calculated for the normalized spectra and used for imaging as shown in FIG. 34c for the first PC score shown in FIG. 34d. Interestingly, this PC score clearly contains all the major peaks of R6G and thereby higher intensity of the depicted data in the PC image on machined area in FIG. 34(c) actually demonstrates better signal quality. However, for the PC calculation, all the investigated points in the imaged area play an equal role, distorting the first PC from bare gold which likely has much lower quality.

To compensate for this drawback of PC imaging, K-mean clustering was used to automatically label the normalized and unlabelled data into two clusters, with the centre of each cluster being its representative spectrum. The K-mean imaging for R6G when different colours (blue and red) are used to represent the different clusters is shown in FIG. 34e. The cluster centres are also shown in FIG. 34f for their corresponding colours in FIG. 34e. Clearly, the boundary of the machined area corresponds to the boundary of the K-mean clusters of the normalized data. Moreover, the fact that the cluster centre corresponding to the machined area more clearly contains all the peaks of R6G compared to the cluster centre for the bare gold indicates that the machined area significantly enhances the quality of the spectra at this concentration. Finally, the scattered points with greater signal quality on the bare gold, but in close proximity to the LINST area (FIG. 34e), indicate the presence and Raman enhancement capability of redeposited gold nanoparticles alone.

### Example 4E—Spectral Amplitude Enhancement

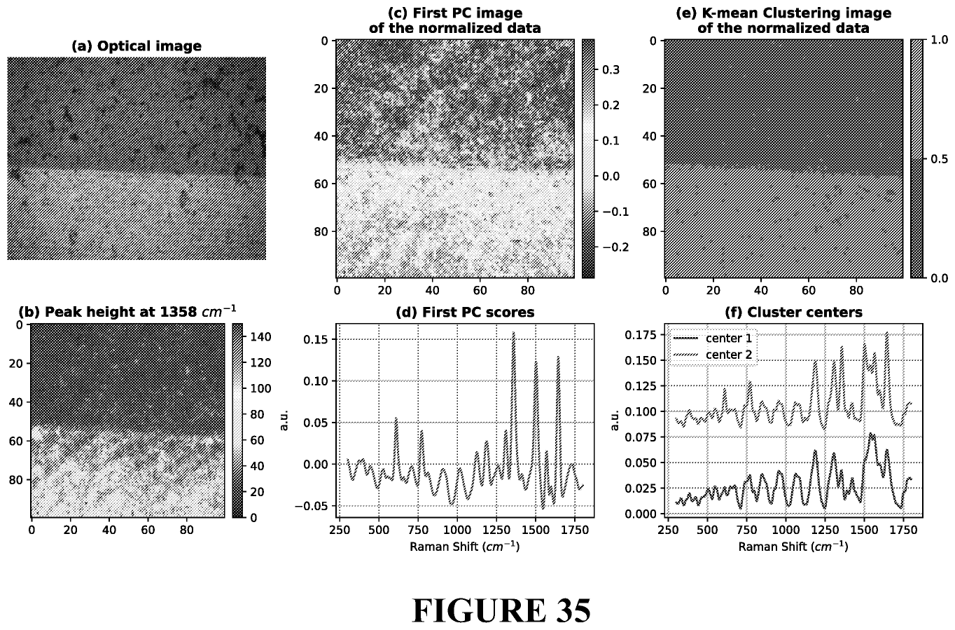

To investigate the amplitude of the obtained spectra, an image of the LINST was taken with the 532 nm laser, 5×microscope object, 25% of ND filter, and R6G concentration of the 10⁻⁶ M. As a higher concentration of R6G was used, the bare gold also produced a Raman signal with acceptable quality, and the difference in this example may be solely due to the amplitude of the obtained signal. In this case, the data which are used for the statistical analyses are not normalized to preserve the amplitude in their result. An optical image corresponding to the Raman hyperspectral imaging area, amplitude of the obtained spectra at 1358 cm⁻¹, first PC image, first PC score, K-mean clustering image, and their corresponding centres are shown in FIG. 35a-f, respectively. In this case, the peak at 1358 cm⁻¹ shows a much stronger signal in the machined area. As seen in FIG. 35d, the first PC score is clearly the expected R6G spectrum, and thus any higher amplitude directly indicates higher intensity of the obtained spectra. Similarly, the K-mean clustering image identified the machined area as a separate cluster and its corresponding centre shows how much higher, on average, the signals in the machined area are in comparison to the bare gold surface.

### Example 4F—SERS Measurements of Preeclamptic (LOPE) EV Suspensions

In a similar approach to the previous chemical testing, hyper-spectral Raman imaging of the LINST substrate of Example 4 was carried out using one of the late onset preeclamptic (LOPE) EV suspensions. The Raman acquisition parameters were: 785 nm laser with 25% of ND filter, 10×microscope object and 0.3 s (300 ms) acquisition time. K-mean clustering was again used to automatically label the data based on its amplitude and signal shape. The optical image of the investigated area, K-mean clustering image, and corresponding cluster centers are shown in FIG. 36a-c.

Clearly, the LINST area produces characteristic EV SERS spectra while the flat gold surface produces little to no signal, with the exception of the LINST-adjacent region containing redeposited gold nanoparticle debris. This EV Raman signal from the regions of flat gold with redeposited nanoparticles is in agreement with the simulations presented in FIG. 32b, where regions of zero curvature still create a hotspot for certain nanoparticle sizes when using the 785 nm laser.

As mentioned above, due to relatively large sizes of even small EVs (30-150 nm) compared to chemical species, and EVs' lack of strong chromophore molecules, on a flat SERS substrate their SERS spectra are much weaker than those of chemical dyes like R6G. Their size also prevents them from fitting perfectly into the nanometric hotspots, in theory resulting in larger EVs producing weaker signals compared to the smaller EVs. Thus, the SERS substrates of the invention which have larger hotspot areas are suitable for EV SERS measurements, in contrast to flat gold surfaces, which provide weak to no Raman spectra for EVs.

Importantly, FIG. 36 shows the availability of strong EV Raman spectra in the region directly adjacent to the LINST area. This appears to be due to the presence of the redeposited gold nanoparticle debris. While their presence only produced moderate Raman enhancement for the R6G in the FIG. 36, the redeposited gold nanoparticle debris produced significantly greater enhancement for EVs. This further illustrates the complex interplay of EVs and the geometry of plasmonic surfaces, and the effect they can ultimately have on SERS measurements.

### Example 5—Classification of Normotensive and Preeclamptic Placental EVs

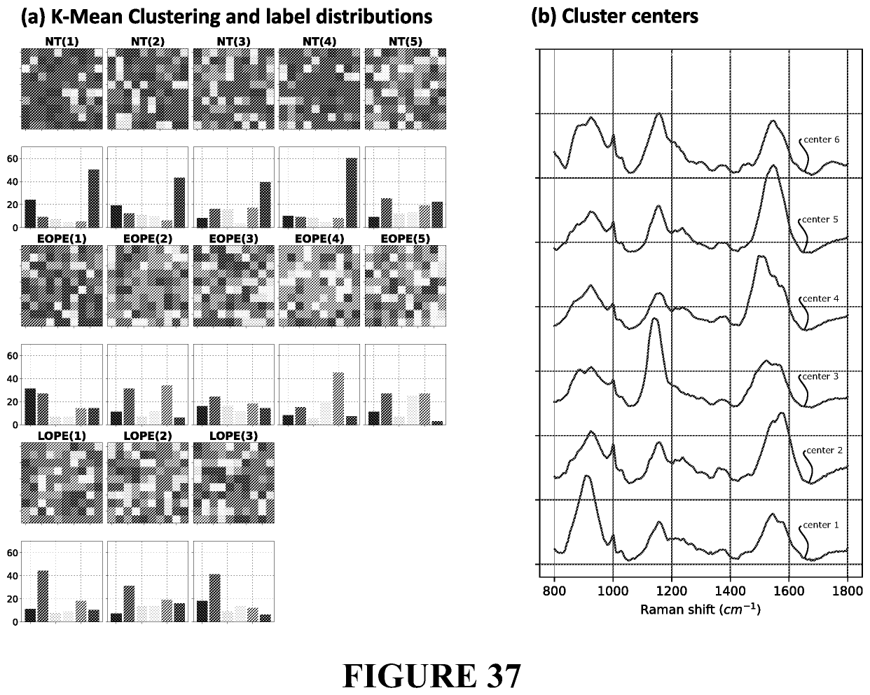

SERS spectra of small EVs isolated from 13 different tissue explant cultures, including 5 normotensive (NT), 5 early onset preeclampsia (EOPE), and 3 late onset preeclampsia (LOPE), were acquired (culture and isolation detailed in methods). For each sample, 100 spectra were obtained over a 10×10 μ rectangular grid on the optimized LINST. To obtain these spectra, an 800 cm⁻¹ to 1800 cm⁻¹ wavenumber range and Raman microscope configurations of 50×microscope object, 785 nm laser, 10 s acquisition time, and 10% ND filter were selected. As the samples in this study are harvested from a tissue explant culture, the EVs were assumed to be significantly heterogeneous, so K-mean clustering was performed spatially for all of the normalized spectra as shown in FIG. 37a. K-mean clustering investigates the abundance of different types of Raman spectra in each sample by automatically labelling the samples based on their shapes and peaks. The centre of each of the clusters are also shown in FIG. 37b.

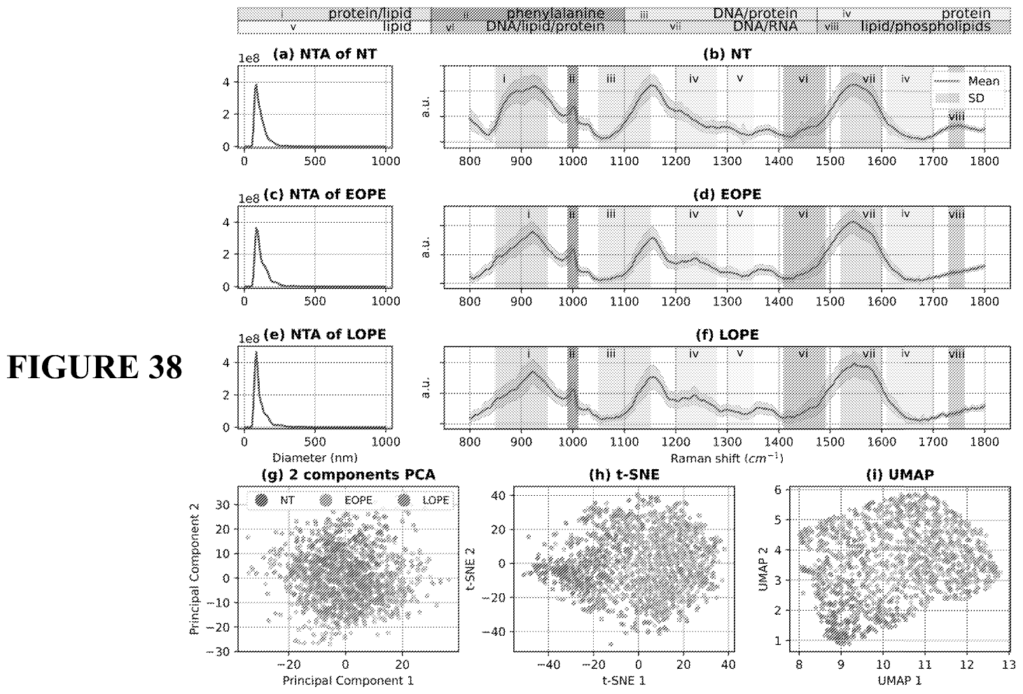

As shown in FIG. 37, there are major differences in the color-coded label distributions between NT, EOPE, and LOPE samples, which represent different EV populations at different laser spots. For instance with reference to FIG. 38a, the 6th label (brown, corresponding to spectral centre 6 in FIG. 37b) dominates much of the NT samples, the 5th label (orange corresponding to spectral center 5 in FIG. 37b) exists in many points of the EOPE samples, and the second label (light blue corresponding to spectral center 2 in FIG. 37b) exists in many points of the LOPE samples. FIG. 37 demonstrates the heterogeneity which can exist even spatially from a single EV sample and will act as a valuable analytical approach as the field of EV SERS continues to grow. By comparison, a more conventional means of reporting EV Raman spectra is shown in 6, where normalized spectra are averaged and presented with standard deviation, removing any information on exact spatial heterogeneity.

In FIG. 38a, the averaged size distributions from nanoparticle tracking analysis (NTA) show the consistent isolation of small EVs (50-200 nm) from differential ultracentrifugation followed by size exclusion chromatography, ensuring that only EVs of similar sizes were compared. In addition, in FIG. 38b, the averaged spectra more clearly demonstrate some key differences between samples types that were initially indicated by the k-means comparison. Here, the average spectrum is presented as the blue line with standard deviation in yellow, with each subtype of biomolecules peaks indicated by corresponding color bands as noted above the spectra. Of particularly interest is the peak at around 1750 cm⁻¹, which is clearly more prominent in the normotensive samples as well as more subtle differences in the shapes and sizes of some peaks. The 1750 cm⁻¹ peak is known to be directly related to the stretching C═O double-bond of esters found in lipids and phospholipids, based on past Raman studies of other biological membranes and lipid reference products.^{77,98} In addition, there appears to be an additional peak at 870 cm⁻¹ in the normotensive samples, which could be caused by several known protein-associated bonds, or the stretching O—C—C—N+ or C4—N+ bonds found in phospholipids.

Given their important and established role in cellular signalling, energy storage, and building of cellular membranes, as well as their clinical association with the vascular wall pathologies, the clear differences in lipid and phospholipid-associated peaks should not be understated. For example, Omatsu et al.¹⁰⁴ demonstrated that injecting phosphatidylserine-phosphatidylcholine artificial micro-vesicles induced a preeclampsia-like disease in mice, while He et al.¹⁰⁵, characterised the maternal blood lipidome and demonstrated that phospholipids including phosphatidylcholines, phosphatidylethanolamines, and ceramides are possible biomarkers for preeclampsia. Confirming the importance of differences in the lipid content of EVs from preeclampsia, Chen et al.¹⁰⁶, have recently published that placental EVs that have vesicle-surface exposed phosphatidyl serine (identified by annexin V binding) are increased in preeclamptic pregnancies. However, the exact role of placental EV-lipid content and its role in normotensive and preeclamptic pregnancies have not been fully elucidated to date. These findings encourage further lipidomic analyses of these types of EVs to better understand the distinct differences that may be present, and if they could be used exclusively as biomarkers outside of SERS analyses.

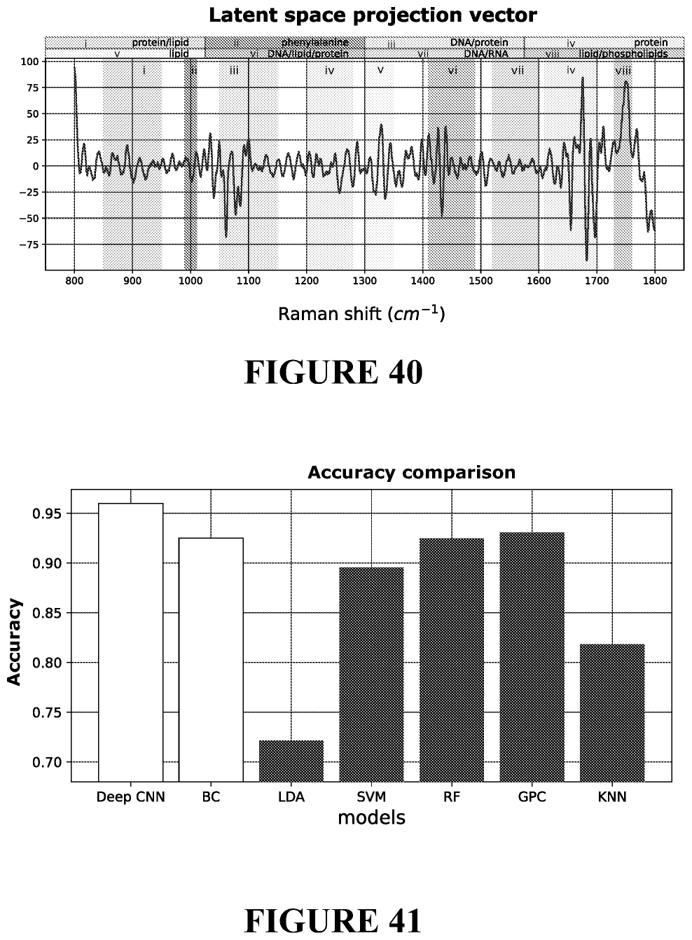

For the sake of further visualizing of all acquired spectra, PCA, t-SNE, and UMAP were used as dimension reduction techniques to embed the high-dimensional pre-processed spectroscopy data into a lower dimension space. The results are presented in FIG. 40.

### Example 6—Classification of Normotensive and Preeclamptic Placental EVs Using Advanced Machine Learning Methods

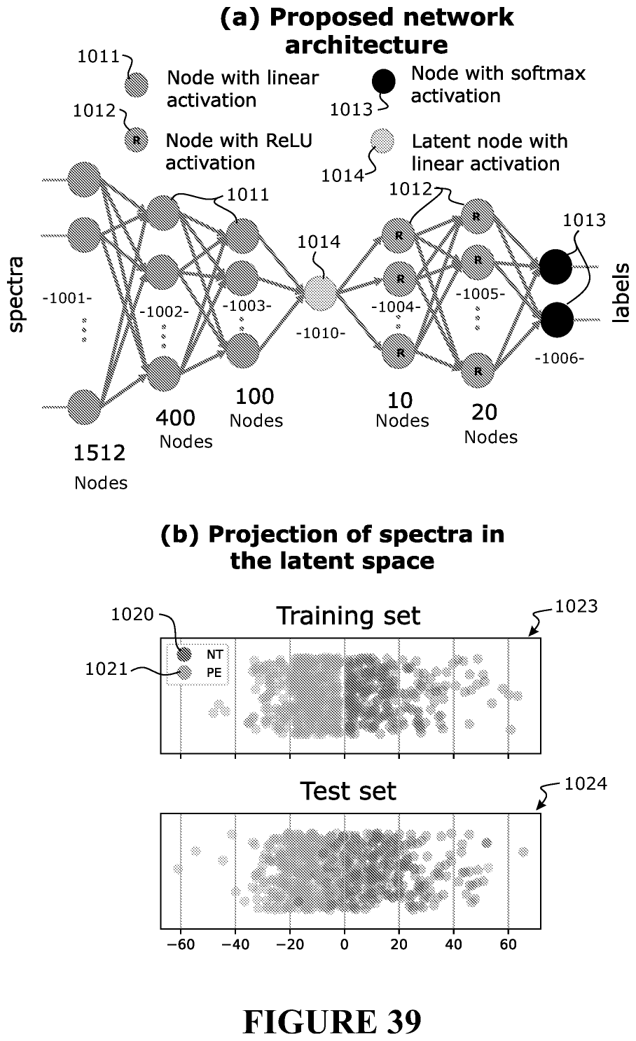

Two advanced machine learning methods were employed to classify the placental EV SERS spectra using neural networks. In the first method, a hybrid autoencoder-inspired architecture was used to first reduce the dimension of the data to one using dense layers with linear activation. Then, nonlinear activated dense layers 1004, 1005 were used to classify the samples with non-linear or ReLU nodes 1012, as shown in FIG. 39a. Additional layer 1006 uses softmax nodes 1013 to generate output data labels. This was done in continuation of the efforts references 107 and 108 for the purpose of simultaneous visualization and classification. The second method is based on a deep convolutional neural network for performing the direct classification over the raw spectra using our previously developed method.

### Example 6A—Bottleneck Classifier and Linear Dimension Reduction

In FIG. 39, the first linear dense layers 1001, 1002, 1003 with linear nodes 1011 seek a particular direction in the Euclidean hyperspace of the data which optimally separates it based on its labels. The main advantage of using this type of network is that the latent space 1010 with node(s) 1014 is explainable such that any obtained values for each of the spectra directly indicates the presence or absence of specific Raman peaks. The distribution of the training and test spectra (when 50 percent of the data is randomly chosen for testing purposes) is shown in FIG. 39b for differentiating between NT particles 1020 and PE particles 1021. As shown, the obtained metric from the training set 1023 is a good way to separate the data based on their labels for both training 1023 and test 1024 sets, as the topology of the training data is recreated with very good precision by the test set. It is important to note that the obtained distribution of the spectra in the latent space is a result of linear transformation, similar to the PCA transformation in FIG. 38. However, while PCA aims to keep the maximum variance of the data in its latent space, the projection in FIG. 39b aims to effectively separate them based on their specific labels within the latent space.

Another important advantage of this type of classification is its ability to avoid potential issues caused by EV heterogeneity. Similar to autoencoder, the presented network is forced to compress the data and thus rejects any randomness in the spectra as much as possible. This important ability was investigated in detail in a prior art network for image denoising and compression.^{109,110}

The calculated direction using the first linear layers 1001, 1002, 1003 is presented in FIG. 40. FIG. 40 shows the relative effect of each spectral value on the overall latent space value calculated by the network. This means that the results of the network are explainable, in the sense that it is possible to state why a particular EV was identified based on the spectra used. This is important for clinical applications. Importantly, as all the first layers 1001, 1002, 1003 are linearly activated, some can easily find the corresponding value of any of the spectra by calculating the dot product of the spectra and the obtained direction and adding the equivalent bias. The obtained result is then classified as the NT if the calculated value is positive and PE if the value is negative, although other labels may be used, and in general any difference between the latent node values may be indicative of different EVs or particles. Interestingly, the obtained direction has two distinct positive peaks around 1330 (cm⁻¹) and 1745 (cm⁻¹), which agrees with FIG. 35, indicating that lipids or phospholipids are projected in the positive side of the latent axis in FIG. 39b. Finally, this custom network achieved more than 92 percent of accuracy of classification between NT and PE.

FIGS. 39 and 40 have explained the use of a bottleneck classifier in an autoencoder to classify EVs and provide a measurable and/or explainable reason for the classification. This is advantageous in clinical settings to establish not only a classification, but also an understandable basis for the classification. The understandable nature is achieved because the plurality of layers 1001, 1002, 1003 between the input spectra are linear layers. As the number of nodes reduces from 1512 to 400 to 100 to the bottleneck (which may be 1, or at least less than 10) the network is forced to generate a latent node(s) which hold all the information about the spectra but which have reduced the dimensionality to the number of latent nodes (e.g. 1). Because the training data forces the bottleneck classifier to classify the spectra (at labels or output nodes 1006) the second portion 1004, 1005 or half of the network can only use those latent features of the bottleneck layer 1010. Non-linear nodes such as ReLU nodes provide a means of achieving this classification. Once trained based on known data (here the data was 50% split into training and test data, but in practise a set of known data for EV spectra will be available, measurable or experimentally found) the network can then be used to test unknown EV spectra as required.

### Example 6B—Bottleneck Classifier and Auto-Encoder

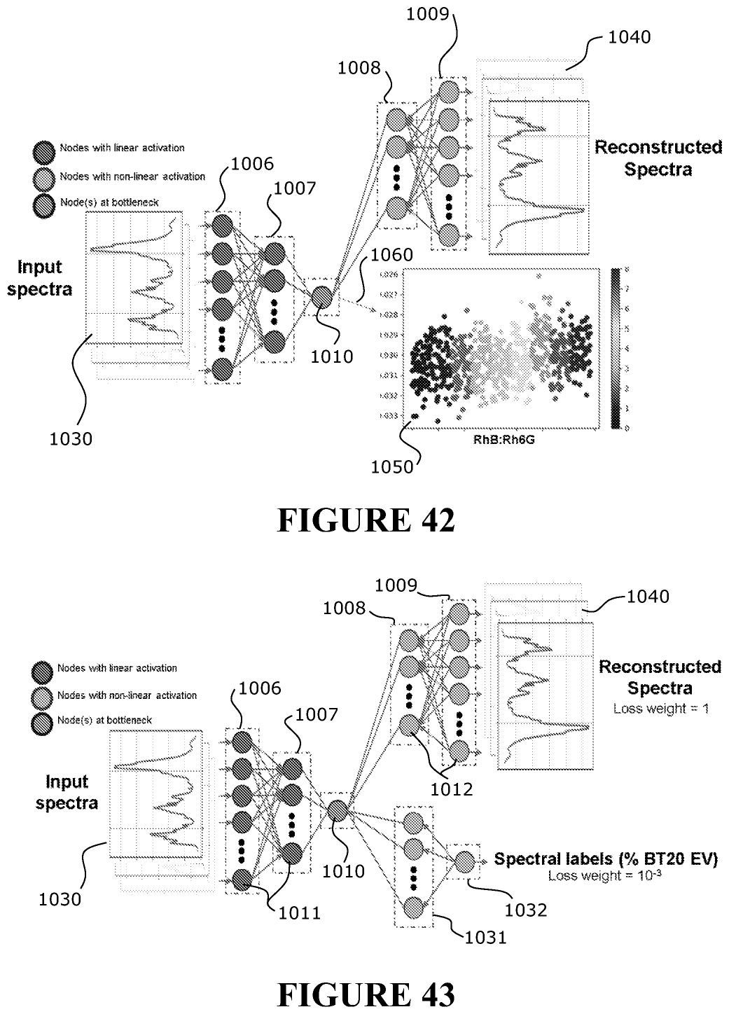

FIG. 42 shows a bottleneck classifier and/or autoencoder. Similar to the embodiment of FIG. 39 a set of preferably linear node 1011 layers 1006, 1007 may be used to reduce the dimensionality of the system (i.e., the number of nodes in a layer) at a bottleneck layer 1010. The input to the first layer 1006 is an EV spectrum 1030, or for training purposes a plurality of EV spectra. The network differs in the second half or portion where one or more, or a plurality of, layers 1008, 1009 (which may have non-linear nodes 1012) recreate or reconstruct the input spectra 1030 to form reconstructed spectra 1040. This means that the latent layer or node(s) 1010 or bottleneck must again have latent features which encode the entire spectra because the spectra are regenerated from only the information contained in the bottleneck nodes 1010. Therefore, by limiting the bottleneck to a small number of nodes we reduce the dimensionality of the spectra to the number of nodes in the bottleneck. The number of nodes at the bottleneck relates to the detection required, for instance to detect a ratio of two EVs a single node would suffice (representing a single value, the ratio), while to distinguish between 3 EVs two nodes would likely be required (it follows that the number of nodes in the bottleneck is preferably one less than the characteristic being tested.

FIG. 43 shows a further improvement to the classifier of FIG. 42. In this case supervised learning is used by the provision of training data into the network. A series of known or labelled spectra is required (either sourced or measured). The second portion of the network, or the portion following the bottleneck layer 1010 now has two portions or outputs. The first is the spectra reconstruction of FIG. 42, while the second is a spectral labelling (which may be similar to FIG. 39) where, a third network portion, generates or is configured to generate a spectral label from the bottleneck node(s). This may be in the form of a simple regression, non-linear classifier, or other set(s) of node layers (1031, 1032). The system is now trained to produce both correct spectral labels and reconstructed spectra, where the spectral labels help to guide and hence provide better separation between EVs. The relative weighting of the second and third networks may be varied (e.g. the loss weight, or which is trained to be more accurate). Advantageously, the reconstructed spectra are weighted greater than the spectral labels, preferably any one of at least 10 times, 100 times or 1000 times greater weighted. This ensures that the labels are not overfit.

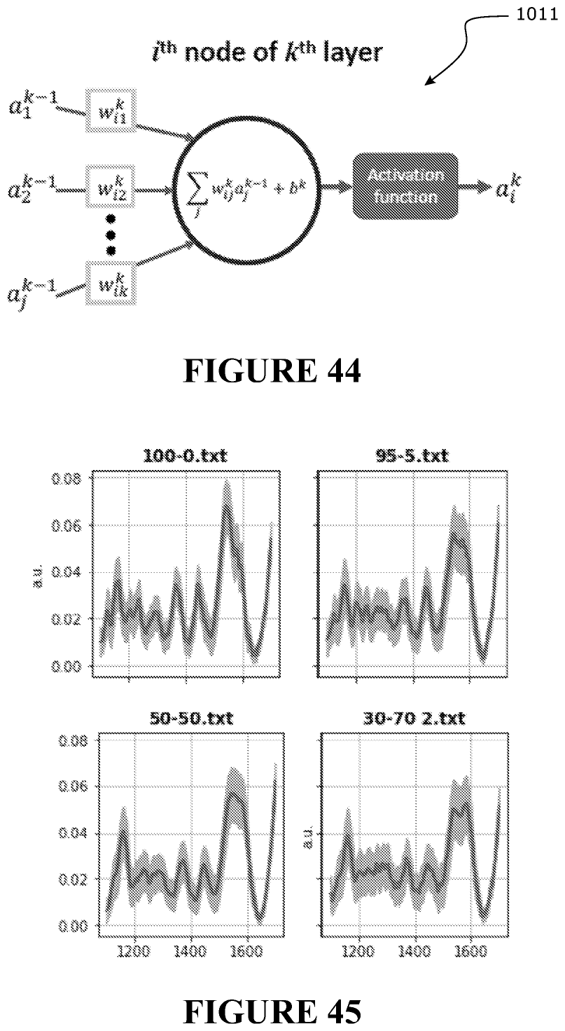

FIG. 44 shows a diagrammatic representation of linear node 1011, which may be used in the first and/or second portion of the networks shown in FIGS. 42 and 43. The choice of the linear activation ensures that the output of each node(s) is a linear combination of its input plus a bias:

$$\\ {a}_{i}^{{}_{} k} = {\sum }_{j} {w}_{ij}^{{}_{} k} {a}_{j}^{{}_{} k - 1} + {b}_{i}^{{}_{} k}$$

Where: w represents the weights associated to the nodes at the previous layer and indices k, i, j indicate respectively the layer, the node whose output is being calculated, and the nodes at the previous layer. a represents the output of a layer and b represents a node's bias.

Therefore, the output of the latent space can be written down as:

$$\\ {a}_{i}^{{}_{} latent} = {\sum }_{j} {𝓏}_{ij}^{{}_{} latent} {x}_{j} + {M}_{i}^{{}_{} latent}$$

In which, a and x are the components of the latent and input data while z and M are the equivalent weights and biases that can be found based on equation of each layer. The weight associated to each node of the latent layer (z) can be consider as a vector with the same dimension as the input vector. That is to say, the latent space (defined by the output of the latent layer), is a linear lower dimensional projection of the data with some biases. This lower dimensional subspace is defined by the vectors zij and biases Mi. For example, in the case where the latent dimension is two, the latent space is a plane indicated by two vectors. As a result, the projected points of the input data is interpretable and their relative positions in the latent space directly indicate the absence or presence of zij vectors in the original data. The selection of the latent space is achieved by selecting the proper vectors zij and biases Mi. These are preferably automatically selected or optimised based on the defined goal and the loss function at the end of decoder layer. In deep neural networks, it is common to use batch normalization layers after each dense or convolution layer. Because these layers are also linear transformations they may be applied or included in the described system without loss of the advantages.

There are currently no rapid, inexpensive, and label-free methods for determining the relative concentration of, for example, FBS EVs within a given sample. The present methods show that surface-enhanced Raman spectroscopy (SERS) can effectively fingerprint foetal bovine serum and other EVs, and after applying a hybrid Autoencoder method or algorithm, the relative FBS EV concentration within a sample can be detected. This is shown using known ratios of Rhodamine B to Rhodamine 6G, then using known ratios of FBS EVs to bioreactor-produced triple-negative breast cancer EVs. This approach could eventually provide several useful applications such as monitoring the integrity of semipermeable membranes within EV bioreactors, ensuring the quality or potency of therapeutic EV preparations, or even determining relative amounts of EVs produced in coculture systems.

The development of EV isolation techniques from cell cultures has revealed several issues with relying on cell cultures as an EV source, such as the complexity of EV samples from patient samples, the lack of biomimicry, and the strong dependence of EV cultures on the use of serum supplementation to support cell growth. These serum supplements contain vast quantities of exogenous EVs that can confound many experimental findings if they are not properly depleted or accounted for with proper controls.

Several researchers have shown that, for example, FBS EVs can have significant effects on several experimental outcomes. Approaches to avoiding these issues include the commercially available EV-depleted serum supplements and bioreactors which allow the use of serum supplements but utilize semi-permeable membranes to prevent serum EVs from passing into the cell culture chambers. However, few techniques have been established to ensure the purity of these EV preparations, with many researchers relying on simple nanoparticle counts, or serum EV controls. A rapid, label-free, and/or inexpensive method of assessing the relative amounts of serum EVs within EV preparations isolated from cell cultures would be advantageous.

Raman spectroscopy has recently emerged as a promising technique for characterizing and identifying different subtypes of EVs from both cell culture and patient samples. By measuring the inelastic scattering of incident light, the chemical bonds of EVs can be recorded as a spectral “fingerprint”, which can then be used for comparative classification purposes. In particular, surface-enhanced Raman spectroscopy (SERS) of EVs could overcome the severe limitations of conventional Raman spectroscopy, such as the need of highly concentrated samples and long signal acquisition times. For SERS, EV Raman spectra are acquired while the EVs are in close proximity to plasmonic surfaces, such that the inelastic scattering is amplified by several orders of magnitude. This not only increases the speed of analysis, but allows the use of much less concentrated samples which is imperative for routine SERS of patient EV samples.

FIG. 45 shows an example set of SERS for different mixtures. In particular the mixtures are for ratios of two particles (RhB and Rh6G, chemical standards—although EVs are the preferably particles (for instance foetal bovine serum (FBS) and MDAMB231). In general, any mixtures of two or more populations of particles with distinct Raman spectra could be used where training data (e.g., a dilution series) is available. This would include other particles of similar size, like lipoproteins, or other biomolecules in solution. Looking at the four example SERS spectra of FIG. 45 we note that it is difficult discern meaningful trend information, likely due to the number of bonds/particles involved in each measurement. The convoluted nature of the spectral peaks, as shown in FIG. 45, compared to chemical species requires more complex methods to process them, including machine learning. Rather than relying on individual peak ratios, machine learning enables the comparison of the latent features within EV Raman spectra, which can be combinations of bonds from hundreds, if not thousands, of different proteins, lipids, nucleic acids, metabolites, and other biomolecules. In addition, it can address the heterogeneity which is inherent in EV samples even from individual cell lines, but particularly relevant for those isolated from patient samples. However even machine learning methods may struggle.

Returning to the networks of FIGS. 42 and 43 we have some stack of dense layers (1001, 1002, 1003, 1006, 1007) in which each layer may be followed by linear activation layers. This first section is known as the encoder as the descending numbers of dimensions (e.g. numbers of nodes) in each consecutive layers compresses the high dimensional data of the input into a lower dimension layer known as latent or code layer. The bottleneck layer 1010 is the minimum dimension used. Preferably it has less than 10 nodes, more preferably less than 5 and most preferably a single node or two nodes. Preferably it has less than 10% of the number of nodes in the first layer, more preferably less than 1% of the number of nodes in the first layer.

The number of nodes in the bottleneck layer can be related to the number of independent parameters in the problem, here ideally the number of nodes or dimension of the bottleneck layer is equal to, or substantially equal to (e.g. within 1, 5 or 10 nodes of), the number of independent parameters. For example, a mixture of ten different chemicals has 9 independent parameters (because, as the sum of all the percentages must be 100 there is always one dependent parameter). For mixtures a general rule is that the number of nodes is one less than the SERS items in the mixture. The bottleneck assists the network to reject non-useful randomness within the data. For example, for the EV mixture problem, the data for each EV subtype is very heterogeneous and the heterogeneity within the EV subtype should be rejected or ameliorated before clustering them based on their mixture. It has been found that the bottleneck is efficient at handling the heterogeneity because the bottleneck reduces the dimensionality or space so that EV heterogeneity is ameliorated, and the network only finds the fundamental differences within the spectra. For example, creating clusters based on mixture ratios.

To construct the decoder, we consider one or two parallel networks. The decoder of FIG. 42 has a stack of dense layers with ascending number of dimensions to finally match its dimension to the output's layer (which is preferably similar or the same as input data because of the autoencoder form). The decoder of FIG. 43 includes the stack or plurality of dense layers (as in FIG. 42) and a second portion or part which is trained to match an output layer 1032 to data's labels (classification or prior knowledge) and/or any scalar number associated to each data (regression).

For the decoder of FIG. 42, in order to extract the highly nonlinear and complex information from the linear projection of data obtained by encoder (i.e., the bottleneck layer), multiple dense layers with rectified liner unite (ReLU) activation are used. To avoid problems such as accuracy saturation and gradient vanishing (especially as gradient needs to reach to the encoder) in the deep decoder network, some skips similar to the one proposed in ResNet may be used. The output preferably has the same dimension as of the input of the network and tries to recreate the input. Regardless of complexity of the decoder architecture, the linear encoder ensures simple and explainable latent space and decoder only tries to retrieve as much information as possible from our linear latent space.

For the decoder of FIG. 43 some layers of non-liner activated dense layer (preferably fewer than the autoencoder network) are used to match the data's labels or any corresponding numerical output. This part can work as a supervised classifier or regression. This network is similar to the bottleneck classifier of FIG. 39 for the purpose of the dimension reduction and classification.

The network can be implemented via TensorFlow in python. Adam optimiser as a variant of stochastic gradient descending is used to train the network while learning rates, α, β1, β2 and ε were chosen to be 0.001, 0.9, 0.999 and 0.00000001, respectively.

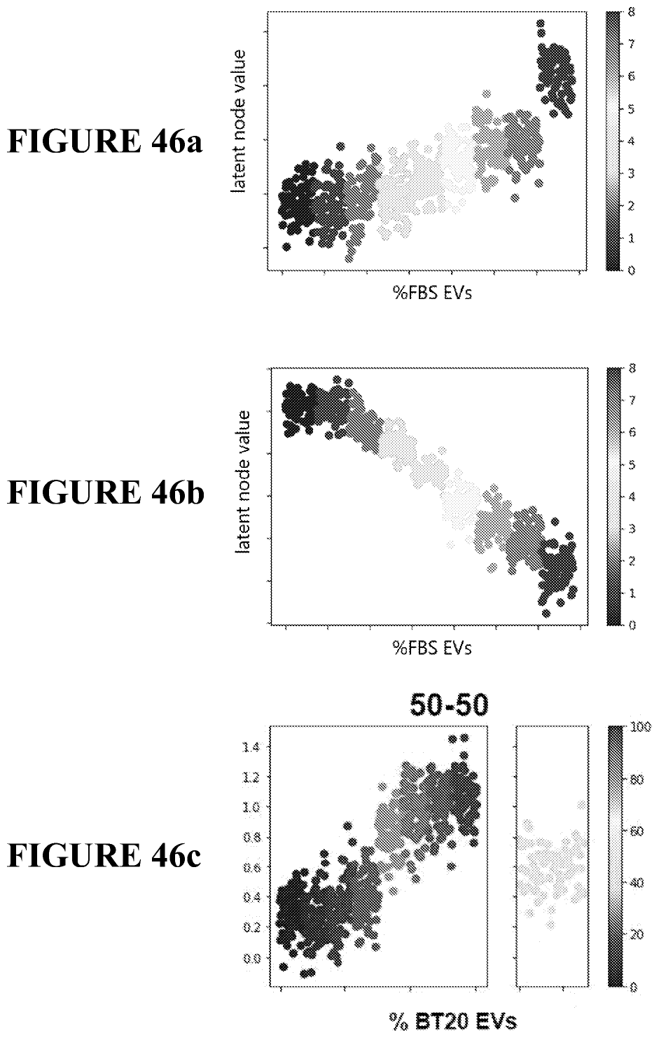

FIG. 46 shows example outputs from the system when training to calculate a percentage of FBS EVs in a sample. The FBS EVs were tested from multiple brands (Gibco™, Moregate™ and Sigma™ and culture media. EV enrichment was performed through a UC, SEC and UF process, although any means suitable to achieved SERS spectra could be used. A range of FBS ratios were prepared, to create enough data (EV spectra) to train either network. The shaded regions, and right had axis indicate the ratio of EVs.

These examples use Rhodamine 6G and Rhodamine 6B aquas solution with 10\hat{ }−6M concentration. These solutions are mixed to achieve different mixture. They are dried onto fabricated SERS substrates (such as, but not limited to the substrates described herein, which use femtosecond laser ablation of gold thin films). Raman measurement was performed using a Horiba LabRam Raman microscope with a 532 nm laser, 50×microscope object and 1 percent of the maximum laser power which is 100 mw. The acquisition time for each spectrum was also chosen to be 1 s only and in total 100 spectra are collected over a 10×10 rectangular grid for each mixture.

FIG. 46a represents a trained output of FIG. 42. A one-dimensional projection is chosen because the change of the Raman spectra for a smoothly changing mixture ratio should only depend on one single number which is the ratio of two chemicals. As just a one single number should uniquely identify each one of the spectra. In other embodiments, such a mixture of a plurality of EVs or particles, a larger number of dimensions may be necessary. The latent node value (the exact value of which is not important) steadily or strictly increases as the percentage of EVs in the sample increases, which demonstrates good classification of the EVs.

FIG. 46b represents a trained output of FIG. 43. Training the network consists of matching input and output for each spectrum until the error is reduced below a threshold or using known neural network training techniques. We expect improvement over 46a because of the additional training and/or information available to the system through the incorporation of the labels. In this example the network 1031, 1032 acts as a regression determining the percentage of R6G in the mixture. The errors generated from both decoders (1031, 1032 and 1008, 1009) are back propagated through the encoder to find a more descriptive latent projection vector to fulfil both output decoder tasks: reconstruction of the spectra 1040 and finding the percentage of the mixture 1032. FIG. 46b shows improved separation of across the FBA ratio (as the y-axis is nominal the positive/negative slope is not important, rather the gradient or steepness, and constant direction is important).

FIG. 46c represents an example use of the system. The network has been trained with eight sets of data or spectra, each set having different EV ratios, shown in the left-hand plot. The amount of training data required will depend on the EVs used and the specificity/accuracy required. This has formed a network where the bottleneck layer produces a clear trend able to classify the percentage of EVs, in this case BT20 EVs. The trained network is then tested on further data, in FIG. 46c a 50% sample of BT20EVs is used (this ratio was not in the training data, but could have been). As shown in the right-hand plot the new data is correctly identified as approximately the mid-point of the training ratios (between 0 and 100 BT20 EVs). If further accuracy was required further training data, or a larger set of training ratios could be used. In some embodiments a trained network based on a dilution series (i.e., a range of known dilutions of EVs).

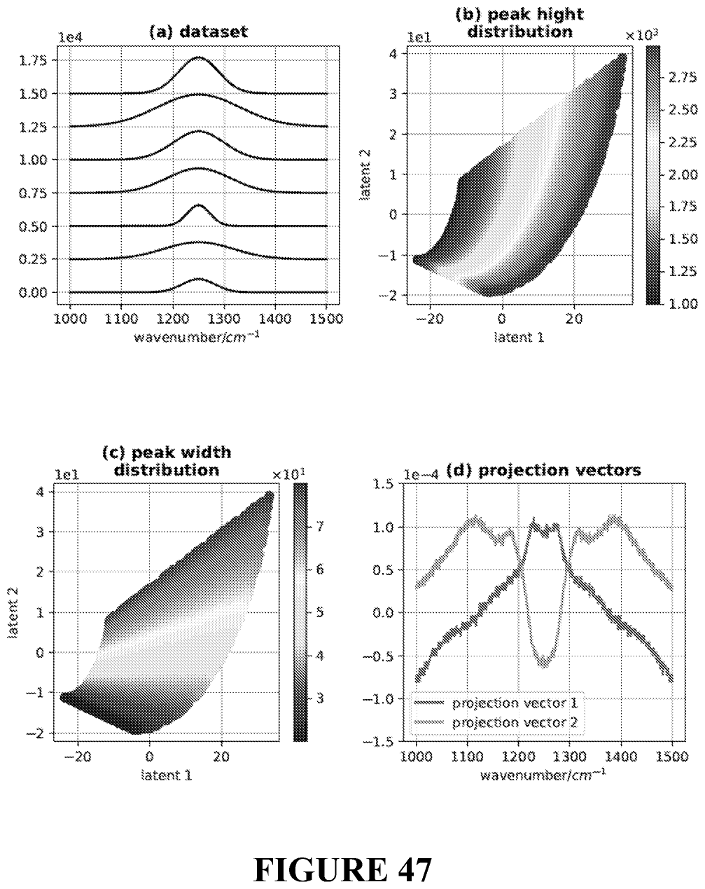

The above examples have described a single node bottleneck layer capable of determining, for instance, relative ratios of EVs in a solution. However, the methods can be applied to more complex or higher dimensional problems. For instance, a mixture of 3 EVs could use a two-dimensional bottleneck layer. FIG. 47 shows an example two-dimensional system, in this case it is a synthetic model generated by modifying the width and height of Gaussian peaks (as shown in FIG. 47a). FIGS. 47b and c plot the synthetic data as a function of the 2-dimensional latent features. The shading identifies the (same) data based on (b) height or (c) width. Because two features are being modified, and of interest, the best result is achieved with two dimensions or projection vectors, and, as shown in FIG. 47(d) the values of both latent numbers, or projection vectors, uniquely identify each plot.

### Example 6C—Deep Convolutional Neural Network (Deep CNN)

Automatic classification was also carried out using a deep convolutional neural network trained using raw the raw EV SERS spectral data. This method leads to improved accuracy over other machine learning methods that are trained using pre-processed data. The classification achieved using this network was not based on the data's baselines or any randomness such as noise or disturbances, and its sensitivity over spectral shifts indicates the classification is based on the spectral peaks' positions. To train this network, the size of the data set for each of the labels (NT and PE) was first increased to 5000 spectra using a data augmentation technique. The augmentation was performed by adding extra Gaussian noise, changing the baselines, small spectral shift and finally linear combination of the spectra within the same label. Then, the network was trained using Adam optimiser as a variant of stochastic gradient descending with learning rate of 10−4. Classification accuracy of 96 percent was achieved using this technique, which is significantly higher than prior art methods. As with almost all deep learning algorithms, this network suffers from uninterpretable classification results. By comparison, the classification methods of Examples 6A, 6B and 6C are more robust or more efficient than conventional machine learning approaches such as linear discriminant analysis (LDA), support vector machine (SVM), Random forest (RF), Gaussian process classifier (GPC), or k-nearest neighbours (KNN). FIG. 41 shows a comparison of the described custom Deep Convolutional Neural Network (CNN) and Bottleneck Classifier (BC) algorithms and conventional machine learning classifiers. Both methods provide strong accuracy, and the bottleneck classifier also enables interpretation of the result.

Given the abundance and availability of placental EVs within maternal circulation, the invention shows promise for placenta-derived EV lipidomic analyses, as well as the use of EV SERS for preeclampsia diagnostic and/or monitoring applications. While this may be possible using the total EV population within the maternal blood, a more specific approach could also be possible by isolating the placenta-specific EVs based on known surface antigens, such as placental alkaline phosphatase or other antigens such as trophoblast glycoprotein/5T4. This could be done either directly on the SERS substrate, which would require compensation for the ligand-specific Raman spectral contribution, or prior to SERS analysis using microfluidic or other EV-capture systems, followed by elution or release.¹¹¹⁻¹¹²

Although this invention has been described by way of example and with reference to possible embodiments thereof, it is to be understood that modifications or improvements may be made thereto without departing from the scope of the invention. The invention may also be said broadly to consist in the parts, elements and features referred to or indicated in the specification of the application, individually or collectively, in any or all combinations of two or more of said parts, elements or features. Furthermore, where reference has been made to specific components or integers of the invention having known equivalents, then such equivalents are herein incorporated as if individually set forth.

Any discussion of the prior art throughout the specification should in no way be considered as an admission that such prior art is widely known or forms part of common general knowledge in the field.

The following additional paragraphs define further embodiments of the present disclosure:

Clause 1. A surface-enhanced Raman spectroscopy device, comprising:
a cured substrate having a plurality of depressions,
a Raman signal-enhancing material disposed on the substrate, such that the material also has a plurality of depressions.

Clause 2. A SERS device comprising a plastics layer with a plurality of depressions and a Raman signal-enhancing material layer disposed thereupon.

Clause 3. A substrate to enable the classification of Extracellular Vesicles (EVs) the substrate comprising:
a cured layer of plastic having a plurality of depressions,
a Raman signal-enhancing material disposed on the layer of plastic, such that the material also has a plurality of depressions.

Clause 4. A method of manufacturing a SERS device comprising the steps of:
a) washing a positively charged glass slide with alcohol to remove residue,
b) treating the glass slide to make it hydrophilic,
c) diluting a suspension of polystyrene beads,
d) sonicating the suspension of polystyrene beads,
e) dropping a portion of said suspension on the glass slide and spreading the suspension uniformly over the surface of the slide,
f) drying the suspension and slide,
g) pouring a silicone over the slide and baked, resulting in a silicone layer on said suspension and slide,
h) removing said silicone layer from said slide and removing any beads in the silicone,
i) coating the silicone layer with a uniform layer of gold.

Clause 5. A system to identify or classify EVs comprising the steps of:
a) applying prepared EVs to a SERS substrate,
b) using a Raman spectrometer to take a plurality of measurements for each EV sample, the plurality of measurements including spectra,
c) analysis of the measurements, including spectra, to characterise the EVs.

Clause 6. A Surface Enhanced Raman Spectroscopy device comprising a substrate and a base layer, wherein the substrate is characterised by one or more of the following features
i. a local curvature range: [−1,1] μm⁻¹,
ii. a curvature that varies randomly across the substrate with values taken from [−1,1] μm⁻¹,
iii. the presence of nanoparticles distributed randomly across the surface of the substrate.

Clause 7. A method of preparing a Surface Enhanced Raman Spectroscopy device, the method comprising
a) depositing a Raman signal enhancing material as a substrate on a base layer,
b) using a pulsed laser source with a pulse width of less than one picosecond to pattern the surface of the substrate generating a patterned area, and
c) making repeated scans over the substrate with the laser source, with each scan being spatially separate so as to enlarge the patterned area to a desired size and to obtain a substantially homogenously patterned substrate.

Clause 8. The method of clause 7, wherein the laser source is a femtosecond laser.

Clause 9. The method of clause 7 or clause 8, wherein the repeated scans result in scanned lines on the substrate, wherein there is separation between the scanned lines and the method further comprises adjustment of the separation between the scanned lines to match the effective beam waist generated by the laser source.

Clause 10. The method of clause 7, wherein the separation between the scanned lines is between about 0.5 to about 2 times the spot size of the laser.

Clause 11. The method of any one of clauses 7 to 10, wherein the method comprises using a fluence ranging from about 0.05 J/cm² to about 0.5 J/cm².

Clause 12. The method of any one of clauses 7 to 11, wherein the laser in step b) has a scanning speed ranging from about 0.5 to about 1.5 mm/s.

Clause 13. The method any one of clauses 7 to 12, wherein the method comprises using a fluence of about 0.2 J/cm² and a scanning speed of about 1.125 mm/s.

Clause 14. The method of any one of clauses 7 to 13, wherein the separation between the scanned lines is about 2.5 μm.

Clause 15. The method of any one of clauses 7 to 14, wherein depositing a Raman signal enhancing material is carried out by sputter coating or thermal evaporation.

Clause 16. The method of any one of clauses 7 to 15, wherein the laser in step b) generates 140 femtosecond (fs) pulses at a central wavelength of about 800 nm and a pulse repetition rate of about 1 kHz.

Clause 17. The device or method of any one of the preceding clauses 6 to 16, wherein the substrate comprises or consists of gold or silver.

Clause 18. The device or method of any one of the preceding clauses 6 to 17, wherein the base layer comprises or consists of a material selected from the group consisting of glass, chromium, silicon, sapphire, silica and germanium.

Clause 19. The device or method of any one of the preceding clauses 6 to 18, wherein the base layer is a dielectric material with a surface roughness of less than about 10 nm.

Clause 20. A system to identify and/or classify extracellular vesicles in a sample, the system comprising the device according to clause 6 or any one of clauses 17 to 19, and machine learning software that compares spectra resulting from use of the device and compares that spectra to a database or training data to classify and identify the spectra.

Clause 21. The system of clause 20 wherein the machine learning software is selected from the group consisting of deep convolutional neural networks, bottle neck classifiers, linear discriminant analysis (LDA), support vector machine (SVM), Random Forest (RF), Gaussian Process Classifier (GPC) and k-nearest neighbours (KNN).

Clause 22. The system of clause 20 or 21, wherein the sample comprises viral, bacterial, cancer and/or placental extracellular vesicles.

Clause 23. The system of any one of clauses 19 to 22, wherein the extracellular vesicles are placental extracellular vesicles, and the system is adapted to distinguish between healthy and pre-eclamptic samples by identifying and/or classifying extracellular vesicles in the sample.

Clause 24. The system of clause 23, wherein the system is adapted to distinguish between samples from subjects with early onset pre-eclampsia and late onset pre-eclampsia.

Clause 25. A method for in vitro diagnosing and/or monitoring the progression of a bacterial infection, viral infection, cancer or pre-eclampsia, the method comprising the identification and/or classification of extracellular vesicles in a sample, by Surface Enhanced Raman Spectroscopy using the device of clause 6 or any one of clauses 17 to 19.

Clause 26. The method of clause 25, wherein the method comprises
a) providing a sample comprising extracellular vesicles
b) contacting the sample with the device of clause 6 or any one of clauses 17 to 19,
c) obtaining one or more Raman spectra of the sample,
d) analysing the one or more Raman spectra using machine learning software to identify and/or classify extracellular vesicles in the sample, and
e) making a determination about the presence and/or progression of a bacterial infection, viral infection, cancer or pre-eclampsia.

Clause 27. The device of clause 6 or any one of clauses 17 to 19 or the system of any one of clauses 19 to 23 for use in a method, preferably an in vitro method, of diagnosing and/or monitoring the progression of a bacterial infection, viral infection, cancer or pre-eclampsia, preferably preeclampsia.

Clause 28. A Surface Enhanced Raman Spectroscopy device of clause 6 substantially as herein described with or without reference to any examples and/or figures

Clause 29. A Surface Enhanced Raman Spectroscopy device of clause 6 substantially as herein described with or without reference to any examples and/or figures.

Clause 30. A method of clause 7 or 25 substantially as herein described with or without reference to any examples and/or figures.

Clause 31. A system of clause 20 substantially as herein described with or without reference to any examples and/or figures.

### REFERENCES

1. T. Nagakubo, N. Nomura and M. Toyofuku, “Cracking open bacterial membrane vesicles”, Frontiers Microbiol., vol. 10, pp. 3026, January 2020.
2. A. T. Tu and A. Tu, Raman spectroscopy in biology: principles and applications (Wiley New York, 1982).
3. M. Jermyn, K. Mok, J. Mercier, J. Desroches, J. Pichette, K. Saint-Arnaud, L. Bernstein, M.-C. Guiot, K. Petrecca, and F. Leblond, “Intraoperative brain cancer detection with raman spectroscopy in humans,” Sci. translational medicine 7, 274ra19-274ra19 (2015).
4. H. Chen, X. Li, N. Broderick, Y. Liu, Y. Zhou, J. Han, and W. Xu, “Identification and characterization of bladder cancer by low-resolution fiber-optic raman spectroscopy,” J. biophotonics 11, e201800016 (2018).
5. P. L. Stiles, J. A. Dieringer, N. C. Shah, and R. P. Van Duyne, “Surface-enhanced raman spectroscopy,” Annu. Rev. Anal. Chem. 1, 601-626 (2008).
6. F. J. García-Vidal and J. Pendry, “Collective theory for surface enhanced raman scattering,” Phys. Rev. Lett. 77, 1163 (1996).
7. J. Pendry, A. Aubry, D. Smith, and S. Maier, “Transformation optics and subwavelength control of light,” science 337, 549-552 (2012).
8. Z. Zeng, Y. Liu, and J. Wei, “Recent advances in surface-enhanced raman spectroscopy (sers): Finite-difference time-domain (fdtd) method for sers and sensing applications,” TrAC Trends Anal. Chem. 75, 162-173 (2016).
9. T. Hutter, S. R. Elliott, and S. Mahajan, “Optical fibre-tip probes for sers: numerical study for design considerations,” Opt. express 26, 15539-15550 (2018).
10. S. Kessentini, D. Barchiesi, C. D'Andrea, A. Toma, N. Guillot, E. Di Fabrizio, B. Fazio, O. M. Marago, P. G. Gucciardi, and M. Lamy de la Chapelle, “Gold dimer nanoantenna with slanted gap for tunable Ispr and improved sers,” The J. Phys. Chem. C 118, 3209-3219 (2014).
11. C. Tira, D. Tira, T. Simon, and S. Astilean, “Finite-difference time-domain (fdtd) design of gold nanoparticle chains with specific surface plasmon resonance,” J. Mol. Struct. 1072, 137-143 (2014).
12. Langer, J. et al. Present and Future of Surface-Enhanced Raman Scattering. ACS Nano 2020, 14, 28-117.
13. Ding, S.-Y.; Yi, J.; Li, J.-F.; Ren, B.; Wu, D.-Y.; Panneerselvam, R.; Tian, Z.-Q. Nanostructure-based plasmon-enhanced Raman spectroscopy for surface analysis of materials. Nature Reviews Materials 2016, 1, 16021.
14. Ferreira, N.; Marques, A.; Águas, H.; Bandarenka, H.; Martins, R.; Bodo, C.; Costa-Silva, B.; Fortunato, E. Label-free nanosensing platform for breast cancer exosome profiling. ACS sensors 2019, 4, 2073-2083.
15. Rojalin, T.; Koster, H. J.; Liu, J.; Mizenko, R. R.; Tran, D.; Wachsmann-Hogiu, S.; Carney, R. P. Hybrid nanoplasmonic porous biomaterial sca old for liquid biopsy di-agnostics using extracellular vesicles. ACS sensors 2020, 5, 2820-2833.
16. Zong, S.; Wang, L.; Chen, C.; Lu, J.; Zhu, D.; Zhang, Y.; Wang, Z.; Cui, Y. Facile detection of tumor-derived exosomes using magnetic nanobeads and SERS nanoprobes. Analytical Methods 2016, 8, 5001-5008.
17. Tian, Y.-F.; Ning, C.-F.; He, F.; Yin, B.-C.; Ye, B.-C. Highly sensitive detection of ex-osomes by SERS using gold nanostar@Raman reporter@nanoshell structures modified with a bivalent cholesterol-labeled DNA anchor. Analyst 2018, 143, 4915-4922.
18. Kim, N.; Thomas, M. R.; Bergholt, M. S.; Pence, I. J.; Seong, H.; Charchar, P.; Todor-ova, N.; Nagelkerke, A.; Belessiotis-Richards, A.; Payne, D. J., et al. Surface enhanced Raman scattering artificial nose for high dimensionality fingerprinting. Nature communications 2020, 11, 1-12.
19. Lee, C.; Carney, R. P.; Hazari, S.; Smith, Z. J.; Knudson, A.; Robertson, C. S.; Lam, K. S.; Wachsmann-Hogiu, S. 3D plasmonic nanobowl platform for the study of exosomes in solution. Nanoscale 2015, 7, 9290-9297.
20. Viehrig, M.; Thilsted, A. H.; Matteucci, M.; Wu, K.; Catak, D.; Schmidt, M. S.; Zór, K.; Boisen, A. Injection-Molded Microfluidic Device for SERS Sensing Using Embedded Au-Capped Polymer Nanocones. ACS Applied Materials & Interfaces 2018, 10, 37417-37425.
21. M. Kahl, E. Voges, S. Kostrewa, C. Viets, and W. Hill, “Periodically structured metallic substrates for sers,” Sensors Actuators B: Chem. 51, 285-291 (1998).
22. L. Petti, R. Capasso, M. Rippa, M. Pannico, P. La Manna, G. Peluso, A. Calarco, E. Bobeico, and P. Musto, “A plasmonic nanostructure fabricated by electron beam lithography as a sensitive and highly homogeneous sers substrate for bio-sensing applications,” Vib. Spectrosc. 82, 22-30 (2016).
23. K. Sivashanmugan, J.-D. Liao, J.-W. You, and C.-L. Wu, “Focused-ion-beam-fabricated au/ag multilayered nanorod array as sers-active substrate for virus strain detection,” Sensors Actuators B: Chem. 181, 361-367 (2013).
24. A. Dhawan, M. Gerhold, and T. Vo-Dinh, “Theoretical simulation and focused ion beam fabrication of gold nanostructures for surface-enhanced raman scattering (sers),” Nanobiotechnology 3, 164-171 (2007).
25. T. Gao, Z. Xu, F. Fang, W. Gao, Q. Zhang, and X. Xu, “High performance surface-enhanced raman scattering substrates of si-based au film developed by focused ion beam nanofabrication,” Nanoscale research letters 7, 399 (2012).
26. M. Cottat, N. Lidgi-Guigui, I. Tijunelyte, G. Barbillon, F. Hamouda, P. Gogol, A. Aassime, J.-M. Lourtioz, B. Bartenlian, and M. L. de la Chapelle, “Soft uv nanoimprint lithography-designed highly sensitive substrates for sers detection,” Nanoscale research letters 9, 1-6 (2014).
27. G. L. Liu and L. P. Lee, “Nanowell surface enhanced raman scattering arrays fabricated by soft-lithography for label-free biomolecular detections in integrated microfluidics,” Appl. Phys. Lett. 87, 074101 (2005).
28. M. Kahraman, P. Daggumati, O. Kurtulus, E. Seker, and S. Wachsmann-Hogiu, “Fabrication and characterization of flexible and tunable plasmonic nanostructures,” Sci. reports 3, 1-9 (2013).
29. Culum, N. M.; Cooper, T. T.; Bell, G. I.; Hess, D. A.; Lagugné-Labarthet, F. Characterization of extracellular vesicles derived from mesenchymal stromal cells by surface-enhanced raman spectroscopy. Analytical and Bioanalytical Chemistry 2021, 1-12.
30. Sivashanmugan, K.; Huang, W.-L.; Lin, C.-H.; Liao, J.-D.; Lin, C.-C.; Su, W.-C.; Wen, T.-C. Bimetallic nanoplasmonic gap-mode SERS substrate for lung normal and cancer-derived exosomes detection. Journal of the Taiwan Institute of Chemical Engineers 2017, 80, 149-155.
31. Hakonen, A.; Svedendahl, M.; Ogier, R.; Yang, Z.-J.; Lodewijks, K.; Verre, R.; She-gai, T.; Andersson, P. O.; Käll, M. Dimer-on-mirror SERS substrates with attogram sensitivity fabricated by colloidal lithography. Nanoscale 2015, 7, 9405-9410.
32. Li, C.; Hu, J.; Jiang, L.; Xu, C.; Li, X.; Gao, Y.; Qu, L. Shaped femtosecond laser induced photoreduction for highly controllable Au nanoparticles based on localized field enhancement and their SERS applications. Nanophotonics 2020, 9, 691-702.
33. Luo, X.; Liu, W.; Chen, C.; Jiang, G.; Hu, X.; Zhang, H.; Zhong, M. Femtosecond laser micro-nano structured Ag SERS substrates with unique sensitivity, uniformity and stability for food safety evaluation. Optics & Laser Technology 2021, 139, 106969.
34. Li, X.; Yuan, G.; Yu, W.; Xing, J.; Zou, Y.; Zhao, C.; Kong, W.; Yu, Z.; Guo, C. A self-driven microfluidic surface-enhanced Raman scattering device for Hg2+detection fabricated by femtosecond laser. Lab Chip 2020, 20, 414-423.
35. Zhou, Z.; Xu, J.; Cheng, Y.; Xu, Z.; Sugioka, K.; Midorikawa, K. Surface-enhanced Raman scattering substrate fabricated by femtosecond laser direct writing. Japanese Journal of Applied Physics 2008, 47, 189.
36. Chen, L.; Zhai, T.; Zhang, X.; Unger, C.; Koch, J.; Chichkov, B. N.; Klar, P. J. Polarization-dependent SERS e'ects of laser-generated sub-100 nm antenna structures. Nanotechnology 2014, 25, 265302.
37. Pavliuk, G.; Pavlov, D.; Mitsai, E.; Vitrik, O.; Mironenko, A.; Zakharenko, A.; Kulinich, S. A.; Juodkazis, S.; Bratskaya, S.; Zhizhchenko, A., et al. Ultrasensitive SERS-based plasmonic sensor with analyte enrichment system produced by direct laser writing. Nanomaterials 2020, 10, 49.
38. Fu, P.; Shi, X.; Jiang, F.; Xu, X. Superhydrophobic nanostructured copper substrate as sensitive SERS platform prepared by femtosecond laser pulses. Applied Surface Science 2020, 501, 144269.
39. Yang, H.; Guan, X.; Pang, G.; Zheng, Z.; Li, C.; Yang, C.; Wang, M.; Xu, K. Femtosecond laser patterned superhydrophobic/hydrophobic SERS sensors for rapid positioning ultratrace detection. Opt. Express 2021, 29, 16904-16913.
40. Guan, X.; Feng, G. Patterned superhydrophobic/superhydrophilic SERS sensors fabricated by femtosecond laser for precise positioning and ultra-sensitive detection. Chemical Physics Letters 2021, 783, 139065.
41. Xu, L.; Liu, H.; Zhou, H.; Hong, M. One-step fabrication of metal nanoparticles on polymer film by femtosecond LIPAA method for SERS detection. Talanta 2021, 228, 122204.
42. Martínez-Calderon, M.; Azkona, J.; Casquero, N.; Rodríguez, A.; Domke, M.; Gómez-Aranzadi, M.; Olaizola, S.; Granados, E. Tailoring diamond's optical properties via direct femtosecond laser nanostructuring. Scientific reports 2018, 8, 1-9.
43. Bonse, J.; Graf, S. Maxwell Meets Marangoni—A Review of Theories on Laser-Induced Periodic Surface Structures. Laser & Photonics Reviews 2020, 14, 2000215.
44. Gnilitskyi, I.; Derrien, T. J.-Y.; Levy, Y.; Bulgakova, N. M.; Mocek, T.; Orazi, L. High-speed manufacturing of highly regular femtosecond laser-induced periodic surface structures: physical origin of regularity. Scientific reports 2017, 7, 1-11.
45. Cao, W.; Jiang, L.; Hu, J.; Wang, A.; Li, X.; Lu, Y. Optical Field Enhancement in Au Nanoparticle-Decorated Nanorod Arrays Prepared by Femtosecond Laser and Their Tunable Surface-Enhanced Raman Scattering Applications. ACS Applied Materials & Interfaces 2018, 10, 1297-1305.
46. Lu, L.; Zhang, J.; Jiao, L.; Guan, Y. Large-scale fabrication of nanostructure on bio-metallic substrate for surface enhanced Raman and fluorescence scattering. Nanomate-rials 2019, 9, 916.
47. Hamad, S.; Bharati Moram, S. S.; Yendeti, B.; Podagatlapalli, G. K.; Nageswara Rao, S.; Pathak, A. P.; Mohiddon, M. A.; Soma, V. R. Femtosecond laser-induced, nanoparticle-embedded periodic surface structures on crystalline silicon for reproducible and multi-utility SERS platforms. Acs Omega 2018, 3, 18420-18432.
48. Szymborski, T.; Stepanenko, Y.; Nici«ski, K.; Piecyk, P.; Berus, S. M.; Adamczyk-Popławska, M.; Kami«ska, A. Ultrasensitive SERS platform made via femtosecond laser micromachining for biomedical applications. journal of materials research and technology 2021, 12, 1496-1507.
49. Saikiran, V.; Dar, M. H.; Kuladeep, R.; Desai, N. R. Ultrafast laser induced subwave-length periodic surface structures on semiconductors/metals and application to SERS studies. MRS Advances 2016, 1, 3317-3327.
50. Bai, S.; Serien, D.; Ma, Y.; Obata, K.; Sugioka, K. Attomolar sensing based on liquid interface-assisted surface-enhanced Raman scattering in microfluidic chip by femtosecond laser processing. ACS Applied Materials & Interfaces 2020, 12, 42328-42338.
51. Shi, B. Recent Advances in the Fabrication of Highly Sensitive Surface-Enhanced Raman Scattering Substrates: Nanomolar to Attomolar Level Sensing. Light: Advanced Manufacturing 2021, 2, 186.
52. Dostovalov, A. V.; Derrien, T. J.-Y.; Lizunov, S. A.; Pºeu£il, F.; Okotrub, K. A.; Mocek, T.; Korolkov, V. P.; Babin, S. A.; Bulgakova, N. M. LIPSS on thin metallic films: New insights from multiplicity of laser-excited electromagnetic modes and efficiency of metal oxidation. Applied Surface Science 2019, 491, 650-658.
53. Rodríguez, A.; Morant-Miñana, M. C.; Dias-Ponte, A.; Martínez-Calderón, M.; Gómez-Aranzadi, M.; Olaizola, S. M. Femtosecond laser-induced periodic surface nanostructuring of sputtered platinum thin films. Applied Surface Science 2015, 351, 135-139.
54. Parellada-Monreal, L.; Castro-Hurtado, I.; Martínez-Calderón, M.; Presmanes, L.; Mandayo, G. G. Laser-induced periodic surface structures on ZnO thin film for high response NO2 detection. Applied Surface Science 2019, 476, 569-575.
55. J. B. Pendry, D. Schurig, and D. R. Smith, “Controlling electromagnetic fields,” science 312, 1780-1782 (2006).
56. P. Mao, C. Liu, G. Favraud, Q. Chen, M. Han, A. Fratalocchi, and S. Zhang, “Broadband single molecule sers detection designed by warped optical spaces,” Nat. communications 9, 1-8 (2018).
57. K. Wegner, P. Piseri, H. V. Tafreshi, and P. Milani, “Cluster beam deposition: a tool for nanoscale science and technology,” J. Phys. D: Appl. Phys. 39, R439 (2006).
58. C. Lee, R. Carney, K. Lam, and J. W. Chan, “Sers analysis of selectively captured exosomes using an integrin-specific peptide ligand,” J. Raman Spectrosc. 48, 1771-1776 (2017).
59. Z. Wang, S. Zong, Y. Wang, N. Li, L. Li, J. Lu, Z. Wang, B. Chen, and Y. Cui, “Screening and multiple detection of cancer exosomes using an sers-based method,” Nanoscale 10, 9053-9062 (2018).
60. E. A. Kwizera, R. O'Connor, V. Vinduska, M. Williams, E. R. Butch, S. E. Snyder, X. Chen, and X. Huang, “Molecular detection and analysis of exosomes using surface-enhanced raman scattering gold nanorods and a miniaturized device,” Theranostics 8, 2722 (2018).
61. T.-D. Li, R. Zhang, H. Chen, Z.-P. Huang, X. Ye, H. Wang, A.-M. Deng, and J.-L. Kong, “An ultrasensitive polydopamine bi-functionalized sers immunoassay for exosome-based diagnosis and classification of pancreatic cancer,” Chem. science 9, 5372-5382 (2018).
62. K. Sivashanmugan, W.-L. Huang, C.-H. Lin, J.-D. Liao, C.-C. Lin, W.-C. Su, and T.-C. Wen, “Bimetallic nanoplasmonic gap-mode sers substrate for lung normal and cancer-derived exosomes detection,” J. Taiwan Inst. Chem. Eng. 80, 149-155 (2017).
63. L. Tirinato, F. Gentile, D. Di Mascolo, M. Coluccio, G. Das, C. Liberale, S. Pullano, G. Perozziello, M. Francardi, A. Accardo et al., “Sers analysis on exosomes using super-hydrophobic surfaces,” Microelectron. Eng. 97, 337-340 (2012).
64. J. Park, M. Hwang, B. Choi, H. Jeong, J.-h. Jung, H. K. Kim, S. Hong, J.-h. Park, and Y. Choi, “Exosome classification by pattern analysis of surface-enhanced raman spectroscopy data for lung cancer diagnosis,” Anal. chemistry 89, 6695-6701 (2017).
65. H. Shin, H. Jeong, J. Park, S. Hong, and Y. Choi, “Correlation between cancerous exosomes and protein markers based on surface-enhanced raman spectroscopy (sers) and principal component analysis (pca),” ACS sensors 3, 2637-2643 (2018).
66. Z. Yan, S. Dutta, Z. Liu, X. Yu, N. Mesgarzadeh, F. Ji, G. Bitan, and Y.-H. Xie, “A label-free platform for identification of exosomes from different sources,” ACS sensors 4, 488-497 (2019).
67. S. Cervo, E. Mansutti, G. Del Mistro, R. Spizzo, A. Colombatti, A. Steffan, V. Sergo, and A. Bonifacio, “Sers analysis of serum for detection of early and locally advanced breast cancer,” Anal. bioanalytical chemistry 407, 7503-7509 (2015).
68. J. Yang, Z. Wang, S. Zong, C. Song, R. Zhang, and Y. Cui, “Distinguishing breast cancer cells using surface-enhanced raman scattering,” Anal. bioanalytical chemistry 402, 1093-1100 (2012).
69. A. Hernández-Arteaga, J. d. J. Z. Nava, E. S. Kolosovas-Machuca, J. J. Velázquez-Salazar, E. Vinogradova, M. José-Yacamán, and H. R. Navarro-Contreras, “Diagnosis of breast cancer by analysis of sialic acid concentrations in human saliva by surface-enhanced raman spectroscopy of silver nanoparticles,” Nano Res. 10, 3662-3670 (2017).
70. V. Moisoiu, A. Socaciu, A. Stefancu, S. D. Iancu, I. Boros, C. D. Alecsa, C. Rachieriu, A. R. Chiorean, D. Eniu, N. Leopold et al., “Breast cancer diagnosis by surface-enhanced raman scattering (sers) of urine,” Appl. Sci. 9, 806 (2019).
71. B. M. Lehrich, Y. Liang, and M. S. Fiandaca, “Foetal bovine serum influence on in vitro extracellular vesicle analyses,” J. Extracell. Vesicles 10, e12061 (2021).
72. P. Zhang, L. Wang, Y. Fang, D. Zheng, T. Lin, and H. Wang, “Label-free exosomal detection and classification in rapid discriminating different cancer types based on specific raman phenotypes and multivariate statistical analysis,” Molecules 24, 2947 (2019).
73. J. Webber and A. Clayton, “How pure are your vesicles?” J. Extracell. Vesicles 2, 19861 (2013). PMID: 24009896.
74. Stewart, M. E.; Anderton, C. R.; Thompson, L. B.; Maria, J.; Gray, S. K.; Rogers, J. A.; Nuzzo, R. G. Nanostructured Plasmonic Sensors. Chemical Reviews 2008, 108, 494-521, PMID: 18229956.
75. Raghu, D.; Christodoulides, J. A.; Christophersen, M.; Liu, J. L.; Anderson, G. P.; Robitaille, M.; Byers, J. M.; Raphael, M. P. Nanoplasmonic pillars engineered for single exosome detection. PloS one 2018, 13, e0202773.
76. Rojalin, T.; Phong, B.; Koster, H. J.; Carney, R. P. Nanoplasmonic approaches for sensitive detection and molecular characterization of extracellular vesicles. Frontiers in chemistry 2019, 7, 279.
77. L.-J. Xu, C. Zong, X.-S. Zheng, P. Hu, J.-M. Feng, and B. Ren, “Label-free detection of native proteins by surface-enhanced raman spectroscopy using iodide-modified nanoparticles,” Anal. chemistry 86, 2238-2245 (2014).
78. H. Shin, D. Seo, and Y. Choi, “Extracellular vesicle identification using label-free surface-enhanced raman spectroscopy: Detection and signal analysis strategies,” Molecules 25, 5209 (2020).
79. Koster, H. J.; Rojalin, T.; Powell, A.; Pham, D.; Mizenko, R. R.; Birkeland, A. C.; Carney, R. P. Surface enhanced Raman scattering of extracellular vesicles for cancer diagnostics despite isolation dependent lipoprotein contamination. Nanoscale 2021, 13, 14760-14776.
80. Jinchao Liu, Margarita Osadchy, Lorna Ashton, Michael Foster, Christopher J Solomon, Stuart J Gibson, Analyst 2017, 142 (21), 4067-4074.
81. Joel Wahl, Mikael Sjödahl, Kerstin Ramser, Applied spectroscopy 2020, 74 (4), 427-438.
82. S. Sun, Q. He, S. Xiao, Q. Xu, X. Li, and L. Zhou, “Gradient-index meta-surfaces as a bridge linking propagating waves and surface waves,” Nat. materials 11, 426-431 (2012).
83. R. F. Oulton, V. J. Sorger, D. Genov, D. Pile, and X. Zhang, “A hybrid plasmonic waveguide for subwavelength confinement and long-range propagation,” nature photonics 2, 496 (2008).
84. A. Yariv, “Coupled-mode theory for guided-wave optics,” IEEE J. Quantum Electron. 9, 919-933 (1973).
85. Clotilde Thery, Kenneth W. Witwer et al., “Minimal information for studies of extracellular vesicles 2018 (MISEV2018): a position statement of the International Society for Extracellular Vesicles and update of the MISEV2014 guidelines.” Journal of Extracellular Vesicles 7(1) 2018.
86. Jiwon Hong, Priscila Dauros-Singorenko, Alana Whitcombe, Leo Payne, Cherie Blenkiron, Anthony Phillips & Simon Swift Analysis of the Escherichia coli extracellular vesicle proteome identifies markers of purity and culture conditions, Journal of Extracellular Vesicles, 8(1) 2019.
87. X. Dai, H. Cheng, Z. Bai, and J. Li, “Breast cancer cell line classification and its relevance with breast tumor subtyping,” J. Cancer 8, 3131 (2017).
88. C. L. Hisey, P. Tomek, Y. N. Nursalim, L. W. Chamley, and E. Leung, “Towards establishing extracellular vesicle-associated rnas as biomarkers for her2+breast cancer,” F1000Research 9, 1362 (2020).
89. S. Knapp, J. Hacker, T. Jarchau and W. Goebel, “Large unstable inserts in the chromosome affect virulence properties of uropathogenic Escherichia coli O6 strain 536 ”, J. Bacteriol., vol. 168, no. 1, pp. 22-30, October 1986
90. A. Nissle, “Die antagonistische behandlung chronischer darmstörungen mit colibakterien”, Med. Klin, vol. 2, pp. 29-33, 1918.
91. G. Sezonov, D. Joseleau-Petit and R. D'Ari, “Escherichia coli physiology in Luria-Bertani broth”, J. Bacteriol., vol. 189, no. 23, pp. 8746-8749, December 2007.
92. P. H. Eilers and H. F. Boelens, “Baseline correction with asymmetric least squares smoothing,” Leiden Univ. Med. Centre Rep. 1, 5 (2005).
93. F. Pedregosa et al., “Scikit-learn: Machine learning in Python”, J. Mach. Learn. Res., vol. 12, pp. 2825-2830, October 2011.
94. Abadi, M. et al. TensorFlow: Large-Scale Machine Learning on Heterogeneous Systems. 2015; https://www.tensorflow.org/, Software available from tensorflow.org.
95. D.-H. Kwon, “Quasi-conformal transformation optics lenses for conformal arrays,” IEEE Antennas Wirel. Propag. Lett. 11, 1125-1128 (2012).
96. M. Kazemzadeh, N. Broderick, W. Xu, and K. Z. Shoshtari, “Design and optimization of broadband and transparent sers based on transformation optics,” in SPIE Micro+ Nano Materials, Devices, and Applications 2019, vol. 11201 (International Society for Optics and Photonics, 2019), p. 112011L.
97. Y. Luo, A. Aubry, and J. Pendry, “Electromagnetic contribution to surface-enhanced raman scattering from rough metal surfaces: a transformation optics approach,” Phys. Rev. B 83, 155422 (2011).
98. M. Kazemzadeh, W. Xu, and N. G. Broderick, “Faster and more accurate time domain electromagnetic simulation using space transformation,” IEEE Photonics J. 12, 1-13 (2020).
99. M. Kazemzadeh, N. Broderick, and W. Xu, “Novel time-domain electromagnetic simulation using triangular meshes by applying space curvature,” IEEE Open J. Antennas Propag. 1, 387-395 (2020).
100. Z. Movasaghi, S. Rehman, and I. U. Rehman, “Raman spectroscopy of biological tissues,” Appl. Spectrosc. Rev. 42, 493-541 (2007).
101. K. B. Johnsen, J. M. Gudbergsson, T. L. Andresen, and J. B. Simonsen, “What is the blood concentration of extracellular vesicles? implications for the use of extracellular vesicles as blood-borne biomarkers of cancer,” Biochimica et Biophys. Acta (BBA)—Rev. on Cancer 1871, 109-116 (2019).
102. L. Van der Maaten and G. Hinton, “Visualizing data using t-SNE”, J. Mach. Learn. Res., vol. 9, no. 11, pp. 2579-2605, 2008.
103. L. McInnes, J. Healy and J. Melville, “UMAP: Uniform manifold approximation and projection for dimension reduction”, arXiv:1802.03426, 2018.
104. Omatsu, K.; Kobayashi, T.; Murakami, Y.; Suzuki, M.; Ohashi, R.; Sugimura, M.; Kanayama, N. Phosphatidylserine/phosphatidylcholine microvesicles can induce preeclampsia-like changes in pregnant mice. Seminars in thrombosis and hemostasis. 2005; pp 314-320.
105. He, B.; Liu, Y.; Maurya, M. R.; Benny, P.; Lassiter, C.; Li, H.; Subramaniam, S.; Garmire, L. X. The maternal blood lipidome is indicative of the pathogenesis of severe preeclampsia. Journal of lipid research 2021, 62 .
106. Chen, Y.; Huang, P.; Han, C.; Li, J.; Liu, L.; Zhao, Z.; Gao, Y.; Qin, Y.; Xu, Q.; Yan, Y., et al. Association of placenta-derived extracellular vesicles with pre-eclampsia and associated hypercoagulability: a clinical observational study. BJOG: An International Journal of Obstetrics & Gynaecology 2021, 128, 1037-1046.
107. Parviainen, E. Deep bottleneck classifiers in supervised dimension reduction. International Conference on Artificial Neural Networks. 2010; pp 1-10.
108. Hinton, G. E.; Salakhutdinov, R. R. Reducing the dimensionality of data with neural networks. science 2006, 313, 504-507.
109. Gondara, L. Medical image denoising using convolutional denoising autoencoders. 2016 IEEE 16th international conference on data mining workshops (ICDMW). 2016; pp 241-246.
110. Cheng, Z.; Sun, H.; Takeuchi, M.; Katto, J. Deep convolutional autoencoder-based lossy image compression. 2018 Picture Coding Symposium (PCS). 2018; pp 253-257.
111. Hisey, C. L.; Dorayappan, K. D. P.; Cohn, D. E.; Selvendiran, K.; Hansford, D. J. Microfluidic aynity separation chip for selective capture and release of label-free ovarian cancer exosomes. Lab on a Chip 2018, 18, 3144-3153.
112. Akbarinejad, A.; Hisey, C. L.; Brewster, D.; Ashraf, J.; Chang, V.; Sabet, S.; Nursalim, Y.; Lucarelli, V.; Blenkiron, C.; Chamley, L., et al. Novel Electrochemically Switchable, Flexible, Microporous Cloth that Selectively Captures, Releases, and Concentrates Intact Extracellular Vesicles. ACS Applied Materials & Interfaces 2020, 12, 39005-39013.
113. Reátegui, E.; van der Vos, K. E.; Lai, C. P.; Zeinali, M.; Atai, N. A.; Aldikacti, B.; Floyd, F. P.; Khankhel, A. H.; Thapar, V.; Hochberg, F. H., et al. Engineered nanointerfaces for microfluidic isolation and molecular profiling of tumor-specific extracellular vesicles. Nature communications 2018, 9, 1-11.
114. Chaiworapongsa, T.; Chaemsaithong, P.; Yeo, L.; Romero, R. Pre-eclampsia part 1: current understanding of its pathophysiology. Nature Reviews Nephrology 2014, 10, 466-480.
115. Han, C.; Wang, C.; Chen, Y.; Wang, J.; Xu, X.; Hilton, T.; Cai, W.; Zhao, Z.; Wu, Y.; Li, K., et al. Placenta-derived extracellular vesicles induce preeclampsia in mouse mod-els. Haematologica 2020, 105, 1686.
116. Gill, M.; Motta-Mejia, C.; Kandzija, N.; Cooke, W.; Zhang, W.; Cerdeira, A. S.; Bastie, C.; Redman, C.; Vatish, M. Placental syncytiotrophoblast-derived extracellular vesicles carry active NEP (neprilysin) and are increased in preeclampsia. Hypertension 2019, 73, 1112-1119.
117. Tannetta, D.; Collett, G.; Vatish, M.; Redman, C.; Sargent, I. Syncytiotrophoblast extracellular vesicles-Circulating biopsies reflecting placental health. Placenta 2017, 52, 134-138.
118 Mincheva-Nilsson, L.; Baranov, V. Placenta-derived exosomes and syncytiotrophoblast microparticles and their role in human reproduction: immune modulation for pregnancy success. American journal of reproductive immunology 2014, 72, 440-457.
119. Chiarello, D. I.; Salsoso, R.; Toledo, F.; Mate, A.; Vázquez, C. M.; Sobrevia, L. Foeto-placental communication via extracellular vesicles in normal pregnancy and preeclampsia. Molecular aspects of medicine 2018, 60, 69-80.
120. Han, C.; Han, L.; Huang, P.; Chen, Y.; Wang, Y.; Xue, F. Syncytiotrophoblast-derived extracellular vesicles in pathophysiology of preeclampsia. Frontiers in physiology 2019, 10, 1236.
121. Salomon, C.; Guanzon, D.; Scholz-Romero, K.; Longo, S.; Correa, P.; Illanes, S. E.; Rice, G. E. Placental exosomes as early biomarker of preeclampsia: potential role of exosomal microRNAs across gestation. The Journal of Clinical Endocrinology & Metabolism 2017, 102, 3182-3194.
122. Pillay, P.; Maharaj, N.; Moodley, J.; Mackraj, I. Placental exosomes and pre-eclampsia: maternal circulating levels in normal pregnancies and, early and late onset pre-eclamptic pregnancies. Placenta 2016, 46, 18-25.
123. Biró, O.; Alasztics, B.; Molvarec, A.; Joó, J.; Nagy, B.; Rigó Jr, J. Various levels of circulating exosomal total-miRNA and miR-210 hypoxamiR in di'erent forms of pregnancy hypertension. Pregnancy hypertension 2017, 10, 207-212.
124. Hemmatzadeh, M.; Shomali, N.; Yousefzadeh, Y.; Mohammadi, H.; Ghasemzadeh, A.; Yousefi, M. MicroRNAs: small molecules with a large impact on pre-eclampsia. Journal of cellular physiology 2020, 235, 3235-3248.
125. Hromadnikova, I.; Dvorakova, L.; Kotlabova, K.; Krofta, L. The prediction of gesta-tional hypertension, preeclampsia and fetal growth restriction via the first trimester screening of plasma exosomal C19MC microRNAs. International journal of molecular sciences 2019, 20, 2972.
126. Shen, L.; Li, Y.; Li, R.; Diao, Z.; Yany, M.; Wu, M.; Sun, H.; Yan, G.; Hu, Y. Placenta-associated serum exosomal miR-155 derived from patients with preeclampsia inhibits eNOS expression in human μmbilical vein endothelial cells. international journal of molecular medicine 2018, 41, 1731-1739.
127. Zhang, J.; Li, H.; Fan, B.; Xu, W.; Zhang, X. Extracellular vesicles in normal pregnancy and pregnancy-related diseases. Journal of cellular and molecular medicine 2020, 24, 4377-4388.
128. Hisey, C. L.; Mitxelena-Iribarren, O.; Martínez-Calderón, M.; Gordon, J. B.; Olaizola, S. M.; Benavente-Babace, A.; Mujika, M.; Arana, S.; Hansford, D. J. A versatile cancer cell trapping and 1D migration assay in a microfluidic device. Biomicrofluidics 2019, 13, 044105.
129. Kruglik, S. G.; Royo, F.; Guigner, J.-M.; Palomo, L.; Seksek, O.; Turpin, P.-Y.; Tatis-che', I.; Falcón-Pérez, J. M. Raman tweezers microspectroscopy of circa 100 nm extra-cellular vesicles. Nanoscale 2019, 11, 1661-1679.
130. Czamara, K.; Majzner, K.; Pacia, M. Z.; Kochan, K.; Kaczor, A.; Baranska, M. Raman spectroscopy of lipids: a review. Journal of Raman Spectroscopy 2015, 46, 4-20.

1. A method of manufacturing a surface-enhanced Raman spectroscopy (SERS) device comprising the steps of
a) depositing a Raman signal enhancing material as a substrate on a base layer,
b) using a pulsed laser source with a pulse width of less than one picosecond to pattern the surface of the substrate generating a patterned area.

2. The method according to claim 1, wherein patterning the surface of the substrate comprises making repeated scans over the substrate with the laser source, with each scan being spatially separate so as to enlarge the patterned area to a desired size and to obtain a substantially homogeneously patterned substrate.

3. The method according claim 2, wherein the repeated scans result in scanned lines on the substrate, wherein there is separation between the scanned lines and the method further comprises adjustment of the separation between the scanned lines to match an effective beam waist generated by the laser source.

4. The method according to claim 3, wherein the separation between the scanned lines is between about 0.5 to about 2 times the effective beam waist generated by the laser source.

5. The method according to any one of claims 1 to 4, wherein the laser source is a femtosecond laser.

6. The method according to any one of claims 1 to 5, wherein the fluence applied to the surface of the substrate is in a range of from about 0.05 J/cm² to about 0.5 J/cm².

7. The method according to any one of claims 2 to 6, wherein the repeated scans are made at a scanning speed ranging from about 0.5 to about 1.5 mm/s.

8. The method according to one of claims 2 to 7, wherein the fluence applied to the surface of the substrate is about 0.2 J/cm2 and the repeated scans are made at a scanning speed of about 1.125 mm/s.

9. The method of any one of claims 3 to 8, wherein the separation between the scanned lines is about 2.5 μm.

10. The method of any one of claims 1 to 9, wherein the Raman signal enhancing material is deposited by sputter coating or thermal evaporation.

11. The method of any one of claims 1 to 10, wherein the laser source in step b) generates 140 femtosecond (fs) pulses at a central wavelength of about 800 nm and a pulse repetition rate of about 1 kHz.

12. The method according to any one of claims 1 to 11, wherein the Raman signal-enhancing material layer comprises or consists of gold or silver.

13. The method according to any one of claims 1 to 12, wherein the Raman signal-enhancing material layer comprises or consists of gold.

14. The method according to any one of claims 1 to 13, wherein the base layer comprises or consists of a material selected from the group consisting of glass, chromium, silicon, sapphire, silica and germanium.

15. The method according to any one of claims 1 to 14, wherein the base layer is a dielectric material with a surface roughness of less than about 10 nm.

16. A surface-enhanced Raman spectroscopy (SERS) device, comprising a base layer and a substrate comprising a Raman signal-enhancing material disposed on the base layer, wherein a surface of the substrate comprises a plurality of features of positive and negative curvature.

17. A SERS device according to claim 16, which comprises a plurality of features of positive and negative curvature in the range: [−1, 1] μm⁻¹.

18. A SERS device according to claim 16 or 17, which comprises a plurality of features of positive and negative curvature with values that vary randomly across the substrate.

19. A SERS device according to any one of claims 16 to 18, further comprising a plurality of nanoparticles.

20. A SERS device according to claim 19, wherein the plurality of nanoparticles is distributed randomly on the surface of the substrate.

21. A SERS device according to any one of claims 16 to 20, wherein the Raman signal-enhancing material comprises or consists essentially of gold.

22. A SERS device according to any one of claims 16 to 21, which has been prepared according to the method of any one of claims 1 to 15.

23. A method for identifying or classifying extracellular vesicles (EVs) in a sample, the method comprising the steps of:
a. applying a sample comprising EVs to a SERS substrate,
b. obtaining one or more Raman spectra for each EV sample,
c. analysing the Raman spectra to identify or classify the EVs.

24. A method according to claim 23, wherein the sample comprising EVs are applied to a SERS device according to any one of claims 16 to 22, or a SERS device prepared according to the method of any one of claims 1 to 15.

25. A method according to claim 23 or 24, wherein the Raman spectra are obtained using an excitation wavelength of 785 nm.

26. A method according to any one of claims 23 to 24, wherein the EVs are identified or classified using one or more of: principal component analysis (PCA); and a neural network.

27. A method according to claim 25, wherein the EVs are identified or classified using a neural network.

28. A method of training a classifier for identification and/or classification of extracellular vesicles, the method comprising the steps of:
Obtaining input data comprising a plurality of Raman spectra;
Training a neural network on the input data, the neural network comprising a plurality of linear layers configured to reduce the dimension of the input data.

29. The method of claim 28 wherein the neural network reduces the dimension of the input data to one.

30. The method of claim 28 or 29 wherein the neural network comprises at least one non-linear layer after the plurality of linear layers.

31. The method of claim 30 wherein the at least one non-linear layer is configured to produce a classification label for the extracellular vesicles.

32. An in vitro method of diagnosing and/or monitoring the progression of a bacterial infection, viral infection, cancer or pre-eclampsia, the method comprising the identification and/or classification of extracellular vesicles in a sample by analysis of one or more SERS spectra of the sample.

33. The method according claim 32, wherein the SERS spectra have been obtained using the device according to any one of claims 16 to 22, or a SERS device prepared according to the method of any one of claims 1 to 15.

34. The method of claim 32, wherein the method comprises
a. providing a sample comprising extracellular vesicles,
b. contacting the sample with the device according to any one of claims 16 to 22, or a SERS device prepared according to the method of any one of claims 1 to 15,
c. obtaining one or more Raman spectra of the sample,
d. analysing the one or more Raman spectra using machine learning to identify and/or classify extracellular vesicles in the sample, and
e. determining the presence and/or progression of a bacterial infection, viral infection, cancer or pre-eclampsia.

35. The method of claim 26 or 27, or any one of claims 32-34, wherein the EVs are classified using a classifier which has been trained according to the method of any one of claims 28-31.

36. A SERS device according to any one of claims 16 to 22, or a SERS device prepared according to the method of any one of claims 1 to 15, for use in an in vitro method of diagnosing and/or monitoring the progression of a bacterial infection, viral infection, cancer or pre-eclampsia.

37. A SERS device according to any one of claims 16 to 22, or a SERS device prepared according to the method of any one of claims 1 to 15, when used in an in vitro method of diagnosing and/or monitoring the progression of a bacterial infection, viral infection, cancer or pre-eclampsia.

38. A kit for analysing extracellular vesicles (EVs) in a sample, the kit comprising a device according to any one of claims 16 to 22, or a SERS device prepared according to the method of any one of claims 1 to 15, and machine learning software that can compare SERS spectra resulting from use of the device to a database or training data to classify and identify the spectra.
